(19) United States
(12) Patent Application Publication
(10) Pub. No.: US 2026/0014362 A1
(43) Pub. Date: Jan. 15, 2026

(54) THERAPEUTIC DELIVERY DEVICE

(71) Applicant: University of Utah Research Foundation, Salt Lake City, UT (US)

(72) Inventors: Dustin Williams, Farmington, UT (US); Nicholas Ashton, Holladay, UT (US); Brian Barnum, Bountiful, UT (US); Alexander Paul Rinn, Salt Lake City, UT (US); Phillip McLaughlin Cullinane, Salt Lake City, UT (US); Andrew J Moore, Riverton, UT (US)

(21) Appl. No.: 19/281,497

(22) Filed: Jul. 25, 2025

Related U.S. Application Data
(63) Continuation-in-part of application No. 18/098,538, filed on Jan. 18, 2023.
Application No. 18/098,538 is a continuation-in-part of application No. 16/994,578, filed on Aug. 15, 2020, now Pat. No. 11,911,584.
Application No. 16/994,578 is a continuation of application No. 16/047,917, filed on Jul. 27, 2018, now Pat. No. 10,744,313.
(60) Provisional application No. 62/537,596, filed on Jul. 27, 2017.

(51) Int. Cl. A61M 39/02; A61M 31/00; A61M 39/10

(52) U.S. Cl. CPC A61M 39/0247; A61M 31/002; A61M 39/1011; A61M 2039/0205; A61M 2039/0276; A61M 2039/0288; A61M 2205/0205; A61M 2205/04; A61M 2209/045; A61M 2210/02

(57) ABSTRACT

Devices and methods for delivering one or more therapeutic agents to an animal are provided. In at least one embodiment, the device includes (1) an at least semi rigid body defining at least one internal reservoir for holding one or more therapeutic agents, (2) a porous rate-controlling membrane located in the internal reservoir that controls the rate of release of the therapeutic agent to the animal; and (3) a fluted extrusion and refilling tube in fluid communication with the reservoir that facilitates inflation or deflation of the internal reservoir with the one or more therapeutic agents where at least the edges of the porous rate-controlling composite membrane are sealed or cladded with at least a portion of the semi rigid body to form a contiguous, fully integrated therapeutic device.

### CLAIM OF PRIORITY

This application is a continuation-in-part of U.S. patent application Ser. No. 18/098,538, filed on Jan. 18, 2023, which is a continuation-in-part of U.S. patent application Ser. No. 16/994,578, filed on Aug. 15, 2020, which issued as U.S. Pat. No. 11,911,584 on Feb. 27, 2024, which is a continuation of U.S. patent application Ser. No. 16/047,917 filed on Jul. 27, 2018, which issued as U.S. Pat. No. 10,744,313 on Aug. 18, 2020, which claims the benefit of U.S. Provisional Application No. 62/537,596 filed on Jul. 27, 2017, all of the above-listed applications are hereby incorporated, by reference, in their entirety, for all purposes.

### BACKGROUND OF THE INVENTION

In the fields of medicine and therapy, there are various advantages and benefits to delivering therapeutic agents to one or more local regions of an animal instead of providing those therapeutic agents systemically to the entire animal. Advantages and benefits include, but are not limited to, achieving high local concentrations of therapeutic where needed most, as opposed to systemic therapy, which produces lower therapeutic concentrations indiscriminately through the tissues of the whole body. Oftentimes, providing the therapeutic agents to exactly the site that needs them allows for much higher (and oftentimes more effective) concentrations of therapeutic agents in target tissues, and those higher concentrations could not be safely achieved in target tissues if administered systemically to the same animal.

Additionally, delivering therapeutic agents locally provides a variety of benefits and advantages, including but not limited to, minimizing side effects in non-target tissues, using less therapeutic agent over the life of delivery (which can reduce costs and/or and morbidity) and effectively delivering the therapeutic agents to the exact site of interest or injury. Several types of therapies that can benefit from local delivery of the therapeutic agents include, but are not limited to, antimicrobial, analgesic, antiseptic, chemotherapeutic, anti-inflammatory, anesthetic management, and other therapeutic agents.

As an example, animals who suffer from open fractures of the extremities are susceptible to high levels of bacterial contamination, especially those bacteria that reside in biofilms. Estimates suggest that greater than 99% of bacteria in natural ecosystems (e.g., soil, dirt, human skin, GI tract, etc.) preferentially dwell in the biofilm phenotype. Mud, dirty water, debris, or other exogenous vectors that harbor biofilms, or high numbers of planktonic bacteria, have potential to contaminate open fracture wounds at the time of trauma and result in infection. However, current antibiotic therapies, which often consist of short-term prophylactic administration, have not been optimized against biofilms. Indeed, almost every, if not every, antibiotic on the market has been optimized against planktonic bacteria. As such, current dosing therapies may not reach sufficient blood levels to effectively eradicate biofilms and their associated bacteria.

Notably, the situation is not unique to open fractures alone. Animals who receive implantable devices including total joint prosthetics, vascular devices, pacemakers, fracture fixation devices, or who undergo surgery in general, are at risk of being contaminated with bacteria, including those in the biofilm phenotype. Oftentimes, those animals that receive implantable devices must have their implantable device adjusted and or replaced, increasing the odds of repeat bacterial infection. Those animals may need repeated doses of therapeutic agents or different therapeutic agents throughout their therapy and treatment and accordingly would benefit from a therapeutic delivery device that can be filled and refilled with one or more therapeutic agents without breaking, leaking, or malfunctioning or creating additional trauma to the animal receiving the therapeutic agent(s). Additionally, animals will benefit from having a therapeutic delivery device that can be inserted into the animal to provide high local concentrations of one or more therapeutics that effectively treats the animal condition. Additionally, the devices of the present disclosure provide the additional benefits that they can be used without the devices breaking, leaking, or malfunctioning in an active animal body and the devices create little to no trauma or damage to the animal that the device is inserted into.

Antimicrobial therapies that are currently in clinical use are limited in their ability to effectively treat and prevent biofilm-related infections, in particular those that accompany the use of implanted devices. Current therapies, including prophylactic antibiotic dosing administered systemically, may not reach intra-wound levels to effectively eradicate biofilm bacteria or high concentration planktonic inocula. It is now well established that bacterial biofilms are resilient and can withstand over 1,000 times the antibiotic concentration compared to their planktonic counterparts. As such, despite antibiotic intervention, biofilms may remain in a contaminated wound site and serve as a reservoir of infection that can endanger the animal and prevent or prolong the animal's full and speedy recovery.

The use of implanted biomaterials (either temporarily or permanently) is commonplace in modern medicine for restoring health and treating disease. One example is the fixation hardware used for repairing fractured bones. Sites containing these and other implantable devices are susceptible to device-related infection. Device-related infections are difficult to treat with clinically available antimicrobial therapies. The characteristic microbiology at the implant surface underlies the unique pathology of device-related infections. Bacteria colonize the foreign surface and evade the host immunity, in part, through secretion of a sticky protective extracellular polymeric (EP) matrix that envelopes the bacteria.

Because of a variety of factors, including but not limited to the changes in the bacterial phenotype, the surface-attached biofilm community may be tolerant of antibiotics and other therapeutic agents up to 1,000 times the concentration typically required to eradicate the metabolically active free-living planktonic forms—concentrations which are toxic to susceptible tissues like the cochlea, liver, and kidneys when delivered systemically. The implant surface thus often serves as a nidus for infection harboring a community of bacteria, which frequently withstand the gold-standard systemic antibiotic treatments employed clinically.

The recalcitrant infections frequently seen in clinical scenarios are a result of the biofilm problem. Since the Type IIIB open fractures were defined in 1984, infection rates (52%) of these fracture types have remained largely unchanged. These high rates of infection hinder surgical outcomes and healing in soldiers, civilians, and animals. There are at least two main reasons why this unacceptably high rate of infection has persisted. First, current clinical therapies were not designed to target biofilms or high inocula of planktonic bacteria. As mentioned, biofilms have significant opportunity to contaminate open fractures or other wound sites at the time of trauma and current antibiotic therapies frequently provide insufficient coverage. Second, current clinical therapies do not maintain sufficiently high doses/concentrations of antibiotic to prevent biofilm-related infection in the traumatic wound site with mechanically impaired vasculature. There exists a need for drug delivery devices that are easy and intuitive to use, thereby reducing the odds of error during use, and can be inserted and/or refilled in the animal numerous times without leaking, breaking, or malfunctioning all the while not causing additional or unnecessary trauma to the patient receiving the therapeutic device.

High energy traumatic wounds and infection outcomes are highlighted in military-related healthcare. In military conflicts, lower extremity injuries are common. Murray outlines that a large percentage of lower extremity combat wounds are complicated by infection. In the military, rates of open fracture formation are much higher compared to the civilian population. For example, 26% of all injuries in soldiers have been reported to be fractures. Of those, 82% were open, with rates of infection reaching as high as 60%. Additional data from Brook Army Medical Center (BAMC) shows that 40% of injured soldiers (26% of whom had orthopedic trauma) from January to June of 2006 received courses of antibiotics. Furthermore, in a group of 25 soldiers who suffered Type IIIB open fractures of the tibia, 77% of their wounds had bacteria present. Taken together, these data indicate that the proposed problem is common and adversely affects wounded warriors and civilian animals, limiting successful surgical outcomes.

Biofilm-related and difficult-to-treat infections are of ever-growing concern across a broad spectrum of healthcare-related practices. Bacteria in planktonic phenotypes can either contaminate a wound or surgical site, then form into a biofilm, or well-established biofilms can contaminate these sites at the time of trauma, injury, or during surgery. Additionally, prior devices and methods had various disadvantages, including but not limited to the device rupturing or failing due to increased forces being applied to the device while in the animal or during removal of the device, joints or stress points of the device failing, the path of the therapeutic agent or compound being severely restricted or blocked when the device is rotated or kinked both while inside the animal or during removal of the device and the device not being able to fully deflate and remove all the therapeutic agents or compounds from the device and refill the device with one or more new therapeutic agents or compounds that can continue to treat the animal and the device being caught on the animal during insertion, removal and use, causing damage to the animal the device was inserted into. Accordingly, there exists a need for an improved therapeutic delivery device that solves the problems and limitations of the above and provides various additional benefits and advantages, all of which are accomplished by the devices of the present disclosure as described herein.

### SUMMARY OF THE INVENTION

The various embodiments of the present disclosure provide for, among other things, devices and methods that provide local, diffusion-based delivery of therapeutic agents or compounds (e.g., antibiotics or other drugs) to manage local infections or other conditions requiring one or more therapeutic agents.

In at least some of the embodiments of the present disclosure, the devices of the present disclosure are designed to be placed in a cavity or soft tissue adjacent to/near the desired treatment zone of the animal. At least one embodiment of the present disclosure provides an improved therapeutic (e.g., antimicrobial or other therapeutic agent) delivery device containing an at least semi rigid body defining at least one internal reservoir for holding one or more therapeutic agents that are to be delivered to an animal, a porous rate-controlling membrane located in the internal reservoir that controls the rate of release of the therapeutic agent to the animal and a fluted extrusion connected to a refilling tube in fluid communication with the reservoir that facilitates inflation or deflation of the internal reservoir with the one or more therapeutic agents. In at least some of the embodiments, the fluted extrusion tube can have one or more flutes depending on the size of the device and the therapeutic agent that is delivered.

In at least one aspect of one embodiment, at least the edges of the porous rate-controlling composite membrane are sealed or clad with at least a portion of the semi rigid body to form a contiguous, fully integrated therapeutic device. The devices of the present disclosure can be used as a standalone product to expedite healing and treat or prevent infection and trauma to wounds and surgical sites.

In another embodiment, a therapeutic delivery device is provided. The device has an at least semi rigid body, having one or more monotonically tapered suture anchors, and defining at least one internal reservoir for holding one or more therapeutic agents that are to be delivered to an animal, a porous rate-controlling composite membrane located in the internal reservoir that controls the rate of release of the therapeutic agent to the animal and a single, bi or tri-fluted extrusion refilling tube in fluid communication with the reservoir that facilitates inflation and/or deflation of the internal reservoir with the one or more therapeutic agents, where at least the edges of the porous rate-controlling composite membrane are sealed or cladded with at least a portion of the semi rigid body.

In at least one aspect of at least one embodiment of the present disclosure, the device includes a single fluted extrusion, bi-fluted extrusion or tri-fluted extrusion, which is heat sealed, radiofrequency (RF) welded, ultrasonic welded, or solvent welded to the at least semi rigid body to form a contiguous, fully integrated therapeutic device.

In at least one aspect of at least one embodiment of the present disclosure, the device further comprises a lock fitting and/or lock connector.

In at least one aspect of at least one embodiment of the present disclosure, the device further comprises a trocar.

In at least one aspect of at least one embodiment of the present disclosure, the porous rate-controlling composite membrane is comprised of polyether sulfone.

In at least one aspect of at least one embodiment of the present disclosure, the porous rate-controlling composite membrane is comprised of fibrous polyethylene terephthalate.

In at least one aspect of at least one embodiment of the present disclosure, the porous rate-controlling composite membrane is comprised of polyether sulfone and a fibrous polyethylene terephthalate.

In another embodiment of the present disclosure, the device has an at least semi rigid body having one or more dimples or channels and one or more tapered suture anchors and the semi rigid body defines at least one internal reservoir for holding one or more therapeutic agents that are to be delivered to an animal, a porous rate-controlling composite membrane located in the internal reservoir that controls the rate of release of the therapeutic agent to the animal.

In at least one aspect of at least one embodiment of the present disclosure, at least the edges of the porous rate-controlling composite membrane are sealed with at least a portion of the semi rigid body to form a contiguous, fully integrated therapeutic device.

In at least one aspect of at least one embodiment of the present disclosure, the at least semi rigid body includes one or more dimples and/or channels that allow the path of the therapeutic agents to remain open even if a portion or section of the device is kinked, twisted or contorted upon itself. The one or more dimples and/or channels facilitate and allow the device to filled and refilled with one or more therapeutic agents.

In at least one aspect of at least one embodiment of the present disclosure, the porous rate-controlling composite membrane is comprised of polyether sulfone and a fibrous polyethylene terephthalate.

In at least one aspect of at least one embodiment of the present disclosure, the one or more dimples or channels of the at least semi rigid body are located in close proximity to the end of the device where the one or more therapeutic agents are filled or refilled.

In another embodiment of the present disclosure, a device for delivering one or more therapeutic agents or compounds to an animal subcutaneously is provided. The device provides, among other things, the ability to release high doses of one or more therapeutic agents (e.g., an antibiotic) in a local area, which the data shown and described herein demonstrates is important for effective biofilm eradication and eradication of high numbers of planktonic bacteria. The devices disclosed herein sustain and control the relatively high dose release of the one or more therapeutic agents through a percutaneous exit site that allows the fluid channel to remain open despite movement, contortion and kinking of the device in the animal and allows for effective removal and reloading of the device with new therapeutic agents.

Additionally, the various features of the device and its fully integrated unitary design also prevents the device from failing to function due to external forces on the device, both while various forces are applied to the device when implanted in the animal and during insertion into and removal from the animal receiving the device. The devices fully integrated unitary design of lack of additional components like suture holes also reduce or completely prevent damage to the animal's tissues when the device is inserted into or removed from the animal. The devices shown and described herein are versatile for use with multiple types of therapeutic agents (e.g., antimicrobials) and/or implant systems with a variety of benefits and advantages not seen with other devices.

In at least some embodiments of the present disclosure, the port has a stem that is in fluid communication with a reservoir inserted subcutaneously into an animal in need of one or more therapeutic agents. A rate-controlling membrane is disposed in the reservoir that allows the one or more therapeutic agents to be delivered to the animal at a controlled rate.

In an aspect of at least one embodiment of the present disclosure, a rate-controlling membrane is used to modulate the molecular mobility of the therapeutic compounds, thereby controlling the therapeutic release profile of the one or more therapeutic agents.

Some of the benefits and advantages of the devices disclosed herein include, but are not limited to, providing a fully integrated and unitary body design that has eliminated all joints (including but not limited to head-sealed joints and all adhesive use, including at connection points). This improvement alone provides dramatically greater durability, safety and a significantly improved resistance to damage from forced being applied to the device when the device is in the animal and when the device is inserted and removed from the animal.

The results of experiments shown and described herein show that the devices of the current disclosure can withstand more than 225 N of force, which did not cause failure in the device, whereas prior versions of the device that did not have the features of the devices of the present disclosure failed when about 50 N of force were applied to the devices. The dramatically improved durability and resistance to failure from forces applied to the devices shown and described herein ensure that the device does not fail when being used and removed and inserted. The improvement alone avoids damage to the device that would prevent it from functioning as intended to deliver one or more therapeutic agents and, as a result, will save lives and allow the device to function as intended for a much longer period of time, allowing for extended use of the device.

Additionally, the improvements shown and described herein, including but not limited to, the use of dimples, fibrous materials, foams, channels, and/or one or more flued extrusions, provided the benefits that the device can be emptied fully and refilled completely, as needed, even when the device is contorted, folded on itself or kinked in the animal. Failure of the devices and the inability to completely empty, fill, or refill the devices with proper therapeutic agents could cause serious injury or death to the animal it was inserted into. For example, if the animal receives too much therapeutic agent too quickly or does not receive enough of the therapeutic agent because the device cannot be filled or refilled with new therapeutic agents, the animal would likely be injured, or the therapeutic agent provided would likely be ineffective. The devices disclosed and described herein deliver the right amount of one or more therapeutic agents to the animal, at the right time and place, over extended periods of time.

In an aspect of at least some of the embodiments of the present disclosure, the device includes a single fluted, bi-fluted, or tri-fluted extrusion, which replaces the perforated tube of prior designs. This improvement dramatically reduces or eliminates the problem that prior devices had, which prevented prior devices from deflating completely and/or being refilled with the proper amount of therapeutic agent. A device that cannot be deflated completely and refilled with the correct amount of new therapeutic agents is ineffective or useless for the animal that received the device, would prevent effective treatment and likely cause harm to the animal.

Additionally, in at least some of the embodiments of the present disclosure, the device includes one or more monotonically tapered suture anchors. The tapered suture anchors prevent snagging upon device insertion and removal, which often causes damage to the animal it is inserted into or removed from and potential damage to or failure of the device if the forces applied to the device are great enough.

It should be appreciated that the rate-controlling membranes of the present disclosure can be comprised of any material that is suitable for the particular animal and the particular therapeutic agent(s) provided to the animal. Some examples of materials that could be used for the rate controlling membrane include, but are not limited to, polyester, polypropylene, polyethylene terephthalate, polyvinyl chloride, polyether sulfone, polyvinylidene fluoride, polyvinylidene difluoride, polydimethylsiloxane, silicone, polyethylene, polystyrene, polyvinyl chloride, polytetrafluoroethylene, polyethylene-vinyl acetate, polyacrylate, polystyrene, polyurethane, regenerated cellulose, or cellulose acetate.

In yet another embodiment of the present disclosure, a device for delivering one or more therapeutic agents subcutaneously to an animal in need thereof is provided. The device comprises (1) a port for receiving one or more therapeutic agents that is to be delivered to an animal subcutaneously, the port having a stem and (2) a reservoir in fluid communication with the stem and comprised of at least two areas, the first area being comprised of a more rigid elastic material and the second area being comprised of a less rigid elastic material and (3) a rate-controlling porous or non-porous membrane disposed in the reservoir that allows the one or more therapeutic agents to be delivered to the animal at a controlled rate.

In at least some embodiments of the present disclosure, the reservoir of the devices of the present disclosure can be inserted subcutaneously into an animal in need of the one or more therapeutic agents and filled, emptied, and refilled to contain the correct amount and concentration of one or more therapeutic agents that are to be delivered to the animal subcutaneously, while at least the reservoir of the device is inserted subcutaneously in the animal in need of one or more therapeutic agents. At least the reservoir portion of the device is capable of being inserted subcutaneously into the body of the animal in need of one or more therapeutic agents and a majority of the one or more therapeutic agents can be removed from the reservoir of the device or filled or refilled into the reservoir of the device without the device being removed from the body of the animal in need of the one or more therapeutic agents.

In yet another embodiment, a device for delivering one or more therapeutic agents subcutaneously to an animal in need thereof is provided. The device comprises (1) a port for receiving one or more therapeutic agents that is to be delivered to an animal subcutaneously, the port having a stem; (2) a reservoir in fluid communication with the stem and comprised of a less compliant elastic material abutting a more compliant elastic material at least at the edges of the reservoir, the reservoir being capable of being inserted subcutaneously into an animal in need of the one or more therapeutic agents and filled or refilled to contain the therapeutic agents that are to be delivered to the animal in need thereof; and (3) a rate-controlling porous or non-porous membrane disposed in the reservoir that allows the one or more therapeutic agents to be delivered to the animal at a controlled rate where at least the reservoir portion of the device is capable of being inserted subcutaneously into the body of the animal in need of one or more therapeutic agents and the one or more therapeutic agents can be removed from the reservoir of the device or filled or refilled into the reservoir of the device without the device being removed from the body of the animal in need of the one or more therapeutic agents.

In yet another embodiment of the present disclosure, a device for delivering one or more therapeutic agents subcutaneously to an animal in need thereof is provided. The device comprises (1) a port for receiving one or more therapeutic agents that is to be delivered to an animal subcutaneously, the port having a stem and (2) a reservoir that is comprised of a compliant elastic material and a rate-controlling membrane that allows the one or more therapeutic agents to be delivered to the animal at a controlled rate, wherein the rate-controlling membrane is disposed in at least the center area of the reservoir and a compliant elastic material is disposed in at least the end of the reservoir and the rate-controlling membrane and the compliant elastic material are attached to each other to form at least a semi-continuous layer and the reservoir is capable of being inflated to contain one or more therapeutic agents and deflated to be substantially flat to remove the device from the animal in need thereof or to receive or refill one or more therapeutic agents that are to be delivered to the animal in need thereof, the reservoir being in fluid communication with the stem of the device. In at least some embodiments, at least the reservoir portion of the device is capable of being inserted subcutaneously into the body of the animal in need of one or more therapeutic agents and the one or more therapeutic agents can be removed from the reservoir of the device or filled or refilled into the reservoir of the device without the device being removed from the body of the animal in need of the one or more therapeutic agents.

In at least one aspect of at least one embodiment of the present disclosure, the reservoir is further comprised of a reinforcement or composite membrane located between the more rigid elastic material and the less rigid elastic material of the reservoir, which provides the device with additional strength and reinforcement thereby reducing the chance of device failure or malfunction.

In at least another aspect of at least one embodiment of the present disclosure, the reinforcement or composite membrane is cladded on both sides of the reinforcement or composite membrane with the more rigid clastic material or the less rigid elastic material and the more rigid material and the less rigid material overlap at least a portion of the reinforcement or composite membrane and create a heat seal with the reinforcement membrane in the reservoir, which provides a variety of benefits and advantages, including but not limited to causing the reservoir of the device to be much stronger, allowing the device to be filled and refilled without the device failing or increased risk of the therapeutic agent leaking from the device and harming the animal it is inserted into, removing blunt or sharp ends or corners and providing a tapered end, allowing the device to be removed from the animal and inserted into the animal easier and with less risk of damage to the device itself and the animal. The devices of the present disclosure are free of any barbed and/or glued connections and provide devices with dramatically increased pullout strength and kink and contortion resistance. The devices of the present disclosure also allow some to all of the therapeutic agents to be removed from the device, eliminate all heat-sealed or other failure-prone joints or connections, and incorporate a rate-controlling membrane that is integrated into the device itself for greater stability and function.

In at least another aspect of at least one embodiment of the present disclosure, the more rigid elastic material and the less rigid elastic material sufficiently overlap the reinforcement or composite membrane and create a heat sealed joint of the more rigid elastic material, the less rigid elastic material and the reinforcement or composite membrane at least at the outer edges of the reservoir. Among other things, the more rigid elastic material and less rigid elastic material overlapping and creating a heat seal joint provide structure and rigidity to the device that allows it to be inserted and removed from the animal and the reservoir to be emptied, filled, and refilled more easily and with less risk of failure.

In at least another aspect of at least one embodiment of the present disclosure, at least a substantial portion of both ends of the reservoir is comprised of the more rigid elastic material, and at least a substantial portion of the middle of the reservoir is comprised of the less rigid clastic material.

In at least another aspect of at least one embodiment of the present disclosure, the device does not have a sleeve covering the reservoir, the reservoir is tapered from at least the reservoir to the stem and the largest diameter of the device when the reservoir does not contain any therapeutic agents is the diameter of the port or the stem allowing for, among other things, easy insertion removal of the device from the animal in need of one or more therapeutic agents.

In at least another aspect of at least one embodiment of the present disclosure, the device further comprises one or more monotonically tapered tabs or holes near the edges or outside the reservoir that can be used to fasten the device subcutaneously to the animal in need of the one or more therapeutic agents.

In at least another aspect of at least one embodiment of the present disclosure, the device further comprises at least one tube having holes, wherein the at least one tube is in fluid communication with the stem and is substantially inside the reservoir, and assists with inflation and deflation of the reservoir with therapeutic agents. Among other things, at least one tube allows removal of most, if not all of the therapeutic agents from the device.

In at least one aspect of at least one embodiment of the present disclosure, the rate-controlling membrane has indentations or pores and the device further comprises one or more chemical stabilizers or plasticizers to keep the pores of the rate-controlling membrane open.

In at least one aspect of at least one embodiment of the present disclosure, the chemical stabilizer is, by way of example and not limitation, glycerol.

In yet another embodiment of the present disclosure, a device that includes a port that has a stem that is in fluid communication with a reservoir inserted subcutaneously into an animal in need of the one or more therapeutic agents is provided. At least a substantial portion of both ends of the reservoir is comprised of the more rigid elastic material, and at least a substantial portion of the middle of the reservoir is comprised of the less rigid elastic material and is capable of being inflated and deflated. The port is configured to extend percutaneously from the body to the surrounding environment such that the port is exposed thereby allowing one or more therapeutic agents to be removed from the device or filled or refilled into the device without the device being removed from the body of the animal in need of the one or more therapeutic agents.

In yet another embodiment of the present disclosure, a device comprising a reservoir for a therapeutic agent and configured to be deployed subcutaneously near a surgical site, the reservoir including a membrane for controlled release of the therapeutic agent to the surgical site, a port including a stem, the port in fluid communication with the reservoir and being configured such that the port allows a user to refill the reservoir via the port, and one or more tabs or holes outside the reservoir that can used to fasten the device relative to the surgical site.

In yet another embodiment of the present disclosure, a device comprising a reservoir for receiving a therapeutic agent and configured to be deployed subcutaneously near a surgical site, a rate-controlling membrane disposed in the reservoir for providing a controlled release of the therapeutic agent to the surgical site, a port having a stem in fluid communication with the reservoir, the reservoir including a region configured to receive a fastener, and at least one tube or cylinder with one or more perforations throughout the at least one tube or cylinder that is disposed in the interior of the reservoir and the at least one tube or cylinder being in fluid communication with the stem and reservoir of the device, is provided.

In yet another embodiment of the present disclosure, a device that includes a reservoir that is comprised of a less compliant elastic material abutting a more compliant elastic material is provided. The reservoir being capable of being inflated and deflated and allowing the therapeutic agent to be inserted subcutaneously into an animal in need of the one or more therapeutic agents.

In yet another embodiment of the present disclosure, a device with a rate-controlling membrane that is disposed in the reservoir or attached to the reservoir, where the device allows the one or more therapeutic agents to be delivered to the animal at a controlled rate and the reservoir has a less compliant elastic material abutting a more compliant elastic material, is provided.

In yet another embodiment of the present disclosure, a device with a reservoir that is comprised of at least two different materials, a rate-controlling membrane that allows the one or more therapeutic agents to be delivered to the animal at a controlled rate that is disposed in at least the center area of the reservoir and a compliant elastic material that is disposed in at least the end of the reservoir and rate-controlling membrane and the compliant elastic material being attached to form at least a semi-continuous layer and the reservoir being capable of being inflated to a substantially cylindrical shape and deflated to receive or refill one or more therapeutic agents that are to be delivered to the animal in need thereof, is provided.

In yet another embodiment of the present disclosure, a therapeutic delivery device is provided that includes a multi-lumen tube in the reservoir of the device. During insertion of the device into the animal and also after the device has been positioned in place in the animal, the multi lumen tube of the device prevents twisting, curling, kinking and folding of the device on itself, often at or near the entry site, from causing a blockage that prevents or restricts the flow of the one or more therapeutic agents, which would prevent the device from functioning as intended. The multi-lumen tube also allows the reservoir of the device to be properly filled, emptied, if needed, and refilled with one or more therapeutic agents as intended. The multi-lumen tube also avoids leaving unwanted or unused therapeutic agents in the device and not providing the correct amount of the therapeutic agents in the reservoir because the device could not be properly emptied, filled, or refilled.

In another aspect of at least some of the embodiments of the present disclosure, the multi-lumen tube passes through at least the neck of the device, preventing the blockage of the one or more therapeutic agents, and in at least some embodiments, the tube is somewhat flexible.

Accordingly, the multi-lumen tube allows the device or a portion of the device (e.g., the reservoir) to twist, curl, kink, contort, or fold without partially or completely blocking the flow of the one or more therapeutic agent(s) throughout the device. Among other things, the flexible multi-lumen tube allows the fluid path of the device to remain open and function as intended even if and when the device twists, curls, kinks, or folds over on itself. The multi-lumen tube allows the therapeutic agent(s) to flow through the device as intended, even if the device is under extreme deformation or extreme forces are applied to the device. It should be appreciated that the multi-lumen tube prevents blockage or restricted flow of the one or more therapeutic agent(s) and allows the device to remain functional and operate as intended. It should also be appreciated that the multi-lumen tube provides a variety of benefits and advantages, including but not limited to, allowing the one or more therapeutic agents to travel throughout the device, as intended, and avoid blockages, even when the device is subject to severe deformation and force.

Material characteristics and the cross-sectional shape of the multi-lumen tube determine the ability of the multi-lumen tube to function to transport the one or more therapeutic agents regardless of loading type, magnitude, or orientation of the device.

In another embodiment of the present disclosure, a method of eradicating biofilm and planktonic bacteria in an animal is provided. The method comprises providing a therapeutic delivery device comprised of at least a semi rigid body defining at least one internal reservoir for holding one or more therapeutic agents that are to be delivered to an animal; a porous rate-controlling membrane located in the internal reservoir that controls the rate of release of the therapeutic agent to the animal and a fluted extrusion refilling tube in fluid communication with the reservoir that facilitates inflation or deflation of the internal reservoir with the one or more therapeutic agents, where at least the edges of the porous rate-controlling composite membrane are sealed or cladded with the at least a portion of the semi rigid body to form a contiguous, fully integrated therapeutic device.

In yet another embodiment of the present disclosure, a method of eradicating biofilm and planktonic bacteria in an animal is provided. The method comprises providing a therapeutic delivery device comprised of at least semi rigid body having one or more monotonically tapered suture anchors and defining at least one internal reservoir for holding one or more therapeutic agents that are to be delivered to an animal and a porous rate-controlling composite membrane located in the internal reservoir that controls the rate of release of the therapeutic agent to the animal; and a tri-fluted extrusion refilling tube in fluid communication with the reservoir that facilitates inflation or deflation of the internal reservoir with the one or more therapeutic agents where at least the edges of the porous rate-controlling composite membrane are sealed or cladded with the at least a portion of the semi rigid body; and where the tri-fluted extrusion is heat sealed, RF welded, ultrasonic welded or solvent welded to the at least the semi rigid body to form a contiguous, fully integrated therapeutic device.

In yet another embodiment of the present disclosure, a method of eradicating biofilm and planktonic bacteria in an animal is provided. The method comprises providing a therapeutic device comprised of at least semi rigid body having one or more dimples or channels and one or more tapered suture anchors and defining at least one internal reservoir for holding one or more therapeutic agents that are to be delivered to an animal and a porous rate-controlling composite membrane located in the internal reservoir that controls the rate of release of the therapeutic agent to the animal, where at least the edges of the porous rate-controlling composite membrane are sealed with the at least a portion of the semi rigid body to form a contiguous, fully integrated therapeutic device and where the one or more dimples and or channels of the at least semi rigid body allow the path of the therapeutic agents to remain open even if the device is kinked or twisted upon itself and facilitates in the device being filled and refilled.

In at least one aspect of at least one method of the present disclosure, the device further comprises a lock fitting and lock connector.

In at least one aspect of at least one method of the present disclosure, the device further comprises a trocar.

In at least one aspect of at least one method of the present disclosure, the porous rate-controlling composite membrane of the method is comprised of polyether sulfone.

In at least one aspect of at least one method of the present disclosure, the porous rate-controlling composite membrane of the method is comprised of fibrous polyethylene terephthalate.

In at least one aspect of at least one method of the present disclosure, the porous rate-controlling composite membrane of the method is comprised of polyether sulfone and a fibrous polyethylene terephthalate.

In at least one aspect of at least one method of the present disclosure, the device of the method further comprises one or more dimples or channels of the at least semi rigid body that are located close to the end of the device where the one or more therapeutic agents are filled or refilled.

Other features and aspects of the invention will become apparent by consideration of the following detailed description and accompanying drawings.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1 is a diagram of the original device being used adjacent to a fracture fixation plate secured to bone.

FIG. 2 is a diagram of how biofilm might contaminate an open fracture. Soil, dirt, and/or mud contain tens of billions of bacteria/g of material. The biofilm phenotype predominates in nature. Open fractures are at significant risk of contamination with biofilms at the time of injury.

FIG. 3 is a graph of release curves of common local delivery products compared to sustained antibiotic delivery by the device. Device curves were obtained in-house using a flow chamber. Other release curves were adapted from literature references.

FIG. 4 is a set of photographs of one embodiment of the device, the components, and method of application. Embodiments of the device disclosed herein were tested in an animal model wherein two simulated fracture fixation plates, on which biofilms were grown, were secured to the proximal medial aspect of a tibia. The devices tested were placed over the plates and refilled with antibiotic solution daily for 10 days to maintain antibiotic(s) delivery at the site of interest.

FIG. 5 is a set of images of side-by-side diffusion cells for permeability testing showing ability of rate-controlling membranes to control diffusion of methylene blue (representative compound, at 0.05% in saline) from a donor cell to a receptor cell. (A) Glass PermeGear diffusion cell. (B) Custom 3D printed cells. (C) Multiple cell setup allows for higher throughput membrane permeability testing.

FIG. 6 is a set of graphs illustrating permeability (P) of representative rate-controlling membrane materials. The top line is data from the donor cell; bottom line from the receptor cell. A) Regenerated cellulose membrane used in pilot testing. B) Hydrophobic PVDF membrane with limited diffusion. C) Synder brand PES membrane rated at 30,000 MWC. D) Synder brand PES membrane rated at 300,000 MWCO. E) Hydrophilic PVDF membrane. F) MCE membrane with support material.

FIG. 7 is a photograph of one embodiment of the device materials showing the rate-controlling membrane with a polyester backing (white material) and a polyurethane material (clear) heat-sealed together via impulse sealing.

FIG. 8 is a photograph of one embodiment of the device showing a thermal press unit that heat seals polymeric materials together. The iron can be customized to create various shapes/sizes of a device iteration.

FIG. 9 is a photograph of one embodiment of the device showing a 3D printed mold around which a copper bar can be wrapped to make an iron for heat sealing membrane and polyurethane materials. Mold had dimensions of 2″×2⅛″. FormLabs tough resin was used for printing.

FIG. 10 is a photograph of one embodiment of the device showing the copper iron made using the 3D printed template. Copper bar that was bent around the template had dimensions of ⅜″× 1/16″ and was ground and polished to create a flat face for heat sealing. Copper was soldered to seal the joints and soldered to a copper base plate to create the iron. Solder joints were tested and determined to maintain integrity up to ˜450° F. Notably, the copper iron can also be machined from a copper block to desired dimensions.

FIG. 11 is a photograph of one embodiment of the device showing a copper iron attached to the heating block of a thermal press being brought down on top of materials that are “sandwiched” between a Teflon sheet and a silicone pad to heat seal materials together.

FIG. 12 is a photograph of one embodiment of the device showing a template (paper) used to align the copper iron on the heat block with materials on the thermal press stage. Alignment tabs (four arms) keep the template in place. A silicone pad allows slight compression and improves the heat seal.

FIG. 13 is a photograph of one embodiment of the device showing the MK membrane and polyurethane materials placed on the thermal press stage. Alignment tabs keep the materials in place and maintain alignment with the copper iron.

FIG. 14 is a photograph of one embodiment of the device showing a Teflon sheet (folded back) being placed over MK membrane/polyurethane materials. The rationale for placing a Teflon sheet over the materials that are intended to be heat-sealed is to prevent the materials from being burned by the hot copper iron once it is engaged.

FIG. 15 is a photograph of one embodiment of the device showing a Teflon sheet placed over MK membrane/polyurethane (from FIG. 13). The alignment tabs and outline of the MK membrane can be seen underneath the Teflon sheet. The Teflon sheet had four holes punched out to align with the MK membrane/polyurethane and to keep the Teflon sheet in place. MK membrane and polyurethane can be seen under the Teflon sheet. The materials were “sandwiched” between the Teflon sheet and silicone pad.

FIG. 16 is a photograph of one embodiment of the device showing tabs cut away from the MK membrane after heat sealing the membrane and polyurethane materials together.

FIG. 17 is a photograph of one embodiment of the device showing the heat-sealed materials folded over in preparation for the second heat-sealing step to create the device design.

FIG. 18 is a photograph of one embodiment of the device showing the 3D printed template to create a copper iron for shaping the device. FormLabs tough resin was used to print.

FIG. 19 is a photograph of one embodiment of the device showing copper iron designed to seal a device design out of polyurethane material. The gap was specifically left so that the folded portion of the MK membrane would not be sealed or burned.

FIG. 20 is a photograph of one embodiment of the device showing a Teflon sheet placed over the MK membrane/polyurethane that was folded over in FIG. 17.

FIG. 21 is a photograph of one embodiment of the device showing heat-sealed polyurethane material created in the shape of a device using the iron from FIG. 19. While the MK membrane is a fairly rigid material, the polyurethane makes it so the MK membrane can be incorporated into a tubular-like, device design. Notably, the size of the device and/or membrane can be customized by varying the iron shape.

FIG. 22 is a photograph of one embodiment of the device showing excess polyurethane material trimmed away from edges and a Luer lock connector attached. This Luer lock is used to connect catheter tubing to the device.

FIG. 23 is a photograph of one embodiment of the device connected to catheter tubing and filled with water.

FIG. 24 is a photograph of one embodiment of the device showing a simple representation of how the device is filled with/connected to a syringe. See FIG. 4, which shows the complete setup, including how a needleless connector is placed to allow for draining and filling the device with fluids.

FIG. 25 is a photograph of one embodiment of the device showing two simulated fracture fixation plates secured to the proximal medial aspect of a sheep tibia, and a photograph of the biofilms of MRSA grown on a plate. Each plate contained ˜3×10⁹ CFU/plate.

FIG. 26 is a photograph of the proximal medial aspect of right sheep tibia before being blasted with an air impact device (AID) blast.

FIG. 27 is a photograph of the proximal medial aspect of a right sheep tibia after being blasted with an AID. The AID blast resulted in burst blood vessels/capillaries and soft tissue trauma.

FIG. 28 is a photograph that illustrates the device removal from the secondary site (created by trocar at surgery).

FIG. 29 is a set of photographs representative of surgical/wound sites in comparative study groups taken at a 21-day endpoint. Suture lines in sheep from the device (pouch) group were the only ones to heal.

FIG. 30 is a set of graphs that show colony-forming units (CFU) represented as bioburden per g of tissue. Tissues were collected directly over one of the plates (from FIG. 25), homogenized, and weighed. The device was the only treatment to reduce site bioburden below a 10⁵ threshold, usually regarded as the quantitative metric for infection. Stimulan beads, commonly used to deliver antibiotics locally, served as a nidus of infection and resulted in the worst infection outcome.

FIG. 31 is a graph that shows the summation of site bioburden, including biofilm on site hardware and on bioburden in site tissues. All groups had 21-day endpoints. Positive control group received biofilm-contaminated plates and no antibiotic treatment. Gentamicin-loaded beads contained 120 mg/12 cc of Stimulan beads. Vancomycin was administered for 10 days (2 g/day). Device resided in place for 10 days; antibiotic solution (vancomycin, gentamicin, rifampin) was replenished once daily.

FIG. 32 is a set of images representative of microCT scans. These scans were collected directly underneath a superior, or proximal, simulated fracture fixation plate (from FIG. 25). The “Time=0” bone sample was a piece of native bone that had an osteotomized fracture created with a bone saw but no other treatment. The + control corresponds to sheep treated with biofilms only; intravenous corresponds to sheep treated with IV vancomycin; pouch corresponds to sheep treated with the device; Stimulan corresponds to sheep treated with antibiotic-loaded Stimulan beads. In these images, the roughness correlates with the degree of osteomyelitis: i.e., the rougher the surface, the greater the osteomyelitis, or the more the bone was affected by bacteria.

FIG. 33 is a set of graphs that show the porosities of bone surfaces from each of the sheep groups. Data indicated that bone surfaces of sheep treated with the device were protected against osteomyelitis to a greater degree than sheep that received clinical standards of care or no treatment at all.

FIG. 34 is a set of images representative of microCT scans indicating depth profiles of the osteotomized saw blade (after being in a sheep for 21 days) and depth of osteomyelitis response in bone surfaces. Sheep that were treated with the device had notably less osteomyelitis compared to sheep from other groups.

FIG. 35 is a diagram of a simulated fracture fixation plate on which biofilms are grown and which is surgically placed on bone as in FIG. 25.

FIG. 36 is a rendering of 2×2 cm simulated fracture fixation plate with four holes for bone screw placement.

FIG. 37 is a rendering of a modified CDC biofilm reactor arm designed to hold simulated fracture fixation plates. Two plates are shown at the bottom of the holding arm.

FIG. 38 is a rendering of a modified CDC biofilm reactor lid showing broth inlet ports, air vent port, rectangular slots into which holding arms can be placed, and center port through which a rod is placed to support the reactor baffle.

FIG. 39 is a rendering of a modified CDC biofilm reactor lid with holding arms and simulated fracture fixation plates in place.

FIG. 40 is a photograph of an assembled modified CDC biofilm reactor prepared for autoclaving.

FIG. 41 is a photograph of Tubing C and D connected with straight line barbed connectors and reduction connectors on the ends of Tubing C.

FIG. 42 is a photograph of a full setup of a modified CDC biofilm reactor with broth in carboy, connected tubing, tubing inserted through a peristaltic pump head, and an effluent tube placed in a waste container. Foil is placed over the lid of the reactor in this instance to reduce risk of contamination.

FIG. 43 is a photograph close-up view of tubing, connectors, and air filter connected to Tubing B on reactor lid.

FIG. 44 is a photograph showing an outer right hind sheep limb clipped from ankle region to above the thigh and up to udders.

FIG. 45 is a photograph showing an inner right hind sheep limb clipped from ankle region to above the thigh and up to udders.

FIG. 46 is a photograph of sheep on polyurethane foam mats and a lift table. Left hind limb is secured to a bar with a tie-down so it does not interfere with blast to the right hind limb.

FIG. 47 is a photograph of the lower portion of the right hind limb tied down (not over-tight) to reduce rebound during blast procedure.

FIG. 48 is a photograph of a nozzle of an air cannon approximately 4 inches away from the proximal medial aspect of a tibia.

FIG. 49 is a photograph of a heavy canvas cloth covering sheep limb to prevent wool from spreading throughout the operating room and to provide a barrier from air.

FIG. 50 is a photograph of sheep on a surgical table that is covered with chucks.

FIG. 51 is a photograph of an IV post secured to a surgical table with a C clamp so it is stabilized.

FIG. 52 is a photograph close up of the dB meter microphone near the opening of the AID.

FIG. 53 is a photograph of a side view of the dB meter microphone near the opening of the AID.

FIG. 54 is a photograph of a metal frame built to hold a NEULOG force plate.

FIG. 55 is a photograph of a force plate placed on a metal frame approximately 4″-6″ away from AID to perform testing.

FIG. 56 is a photograph of a cadaveric sheep limb attached to a metal frame. The distance from the nozzle of the AID (threaded portion) to the limb was approximately 4″ (arrow indicates gap). The metal brace (arrow) on the back of the limb provided support to the femur and prevented breakage or ligament damage from occurring.

FIG. 57 is a photograph of the AID and prototype frame system that can be used in the OR to perform the AID discharge. The AID is secured by tie-downs. A metal frame is connected to wooden posts such that a sheep leg can be braced against it.

FIG. 58 is a photograph of a sheep leg (not a live animal) braced against the metal frame. Securing the limb reduces risk of ligament or other damage.

FIG. 59 is a set of photographs that demonstrate creating an incision to expose periosteum (Incision), suturing the subdermal tissue (Suture 1) and dermal layers (Suture 2). Note the cortical bone in the Incision image is visible to allow for periosteum disturbance when necessary.

FIG. 60 is a photograph of a gauge to determine PSI in the AID.

FIG. 61 is a photograph of a representative digital readout of the Reed dB meter.

FIG. 62 is a set of photographs of still shots from AID testing on a cadaveric sheep limb. Note the limb is still intact and attached to the metal stand at all times.

FIG. 63 is a photograph of a suture line before AID procedure.

FIG. 64 is a photograph of a suture line after the AID procedure.

FIG. 65 is a radiograph from specimen (FIG. 62) that showed no signs of gross fracturing or damage to the femur (vertical arrow). The radiolucency seen at the left end of the bone (angled arrow) was damage caused by the bolt used to secure the limb to the metal frame.

FIG. 66 is a photograph of the proximal medial aspect of a right sheep tibia (AP view). Boxed region is approximately where simulated fracture fixation plates reside under the skin. Markers identify surgically-relevant points.

FIG. 67 is a photograph of an incision line exposing the fascia at the proximal medial aspect of a sheep tibia.

FIG. 68 is a photograph of a section of periosteum removed from the proximal medial aspect of tibia.

FIG. 69 is a photograph of simulated fracture fixation plates situated over the proximal medial aspect of a right sheep tibia. Plates/screws are removed after templating to create an osteotomized fracture.

FIG. 70 is a photograph of an osteotomized fracture that runs at a slight angle across the region where a simulated fracture fixation plate resides.

FIG. 71 is a photograph of simulated fracture fixation plates with Stimulan beads placed over them.

FIG. 72 is a photograph of simulated fracture fixation plates with a device placed over them.

FIG. 73 is a photograph of a percutaneous tube protruding through the skin via a secondary site that is created with the trocar.

FIG. 74 is a photograph of sutures closing a surgical site and Stimulan beads are underneath the skin.

FIG. 75 is a photograph of the sutures closing a surgical site and a device is underneath the skin.

FIG. 76 is a photograph of a surgical/wound site at necropsy (was treated with Stimulan beads).

FIG. 77 is a photograph of the surgical/wound site after being cleaned and prepped for sample collection.

FIG. 78 is a photograph of an exposed inferior (distal) plate at necropsy.

FIG. 79 is a schematic of a simulated fracture fixation plate and the sequence in which screws are removed for microbiological analysis.

FIG. 80 is a photograph showing what a surgical site looks like after removing screws and plates.

FIG. 81 is a photograph of a surgical site after tissue that was over the inferior (distal) plate has been removed.

FIG. 82 is a photograph of the site after a bone sample has been collected using a circular trephine.

FIG. 83 is a photograph of the conical tubes showing weights of the tube (with lids on) prior to and after adding tissue samples into them.

FIG. 84 is a photograph of the conical tubes showing what tissue samples look like after being blended.

FIG. 85 is a schematic showing the pattern in which to create a streak plate on an agar surface.

FIG. 86 is a photograph of small test tubes with 900 μl PBS prepped for a 10-fold dilution series.

FIG. 87 is a photograph of agar plates used to plate diluted samples for CFU quantification.

FIG. 88 is an image of one embodiment of the device showing a stress map that indicates regions of the device that are under stress when the device is inflated.

FIG. 89 is an image of one embodiment of the device showing another stress map that indicates regions of the device that are under stress when the device is inflated.

FIG. 90 is an image of one embodiment of the device showing another stress map that indicates regions of the device that are under stress when the device is inflated.

FIG. 91 is an image of one embodiment of the device showing another stress map that indicates regions of the device that are under stress when the device is inflated.

FIG. 92 is an image of one embodiment of the flat shape of the device overlaid with a stress map from modeling the stresses in the final inflated 3-dimensional device of panel FIG. 23. The location of the drug-releasing diffusive membrane (see the dashed box) was positioned within the low stress zones for two reasons: First, the membrane material is non-extensible or inelastic and thus unable to accommodate the stresses of the inflated device; second, this design minimizes the stresses felt on the more delicate membrane thus protecting it from rupture during use.

FIG. 93 is an image of one embodiment of the device showing a 3-dimensional rendering of the device with the inset membrane material (light grey) integrated into a more distensible elastic or yielding material (dark grey).

FIG. 94 is a graph that shows data outlining the force of the 2″ AID discharge opening at 90 PSI and 120 PSI.

FIG. 95 is a picture of backscatter electron images of sheep bones with (top) and without (bottom) therapeutic device treatment. The bone surface with (top) therapeutic device treatment is largely unchanged, whereas the bone surface without (bottom) therapeutic device treatment shows signs of bone desorption, sequestra formation, and moth-eaten bone indicative of osteomyelitis.

FIG. 96 is a drawing of one embodiment of the present disclosure shown and described herein.

FIG. 97 is a drawing of another embodiment of the present disclosure shown and described herein.

FIG. 98 is a drawing of yet another embodiment of the present disclosure shown and described herein.

FIG. 99 is a drawing of yet another embodiment of the present disclosure shown and described herein.

FIG. 100 is a drawing of a trocar, which can be used with at least some embodiments of the present disclosure shown and described herein.

FIG. 101 is another drawing of a trocar, which can be used with at least some embodiments of the present disclosure shown and described herein.

FIG. 102 is another drawing of a trocar, which can be used with at least some embodiments of the present disclosure shown and described herein.

FIG. 103 is a drawing of the deflation assist tube that can be used with at least some embodiments of the present disclosure shown and described herein.

FIG. 104 is another drawing of the deflation assist tube that can be used with at least some embodiments of the present disclosure shown and described herein.

FIG. 105 is another drawing of the deflation assist tube that can be used with at least some embodiments of the present disclosure shown and described herein.

FIG. 106 is a drawing of yet another embodiment of the present disclosure shown and described herein with a deflation assist tube shown in the device reservoir.

FIG. 107 is a drawing of yet another embodiment of the present disclosure shown and described herein.

FIG. 108 is a drawing of yet another embodiment of the present disclosure shown and described herein.

FIG. 109 is a drawing of yet another embodiment of the present disclosure shown and described herein.

FIG. 110 is a schematic of how the cladding material bridges the less compliant and more compliant materials by at least some embodiments of the present disclosure.

FIG. 111 is a graph showing the cumulative biofilm burden remaining in various surgical/treatment sites of sheep inoculated with mono-microbial biofilms of P. aeruginosa or S. aureus and treated with the therapeutic device or clinical standards of care. Inoculation amount indicates the number of bacteria that were on simulated fracture fixation plates at the time of surgery (the inoculation level). Bioburden is the total aggregate of bacteria quantified in the surgical site (soft tissue, hard tissue, and hardware) at necropsy.

FIG. 112 is a graph showing the cumulative biofilm burden remaining in various surgical/treatment sites of sheep inoculated with poly-microbial biofilms of P. aeruginosa and S. aureus and treated with a therapeutic device or clinical standards of care. P. aeruginosa and S. aureus are isolated using selective media and thus presented separately but show the same trend:

when treated with the device shown and described herein, bioburden is reduced to a greater degree (˜1 log₁₀ unit) than standards of care (VT is vancomycin and tobramycin powders; STIM is Stimulan® beads loaded with vancomycin and tobramycin). Inoculation indicates the number of bacteria that were on the plates at the time of surgery (the inoculation level). Bioburden is the total aggregate of bacteria quantified in the surgical site (soft tissue, hard tissue, and hardware) at necropsy.

FIG. 113 is a rendering of one embodiment of the device showing a multi-lumen deflation assist tube that reduces the risk of kinking.

FIG. 114 is a rendering of one embodiment of the device showing the cross section of a multi-lumen deflation assist tube that reduces the risk of kinking.

FIG. 115 is a rendering of the cross-section of a deflation assist tube with coiled material that reduces the risk of kinking.

FIG. 116 is another rendering of one embodiment of the device showing a deflation assist tube with coiled material that reduces the risk of kinking of the tube.

FIG. 117 is another rendering of one embodiment of the device showing a deflation assist tube with coiled material that reduces the risk of kinking.

FIG. 118 is a rendering of various iterations of the device of the present disclosure, including alternative or older versions of the device. The two devices shown at the top of page of FIG. 118 are older versions of the device that included a sleeve, a contiguous rate controlling membrane and blunt ends, among other things. The two devices directly below the two devices shown at the top of the page of FIG. 118 show the devices of one or more embodiments of the present disclosure and include some of the improvements of the new device design, including but not limited to a non-contiguous rate controlling membrane, a tapered end and elimination of blunt ends that could harm the animal and device during insertion and remove into the animal, elimination of the protective sleeve of the device, kink resistant tube that allows most if not all of the therapeutic agent(s) to be removed, heat sealed joins that add structure, rigidity and allow the device to be filled, refilled and reused numerous times.

FIG. 119 is a table showing the results of experiments for sheep groups, biofilm inocula (monomicrobial or polymicrobial), treatments, dosing, and endpoints using the inventions of the present disclosure.

FIG. 120 is a drawing in accordance with at least one embodiment of the device of the present disclosure that shows the stem area of the device past the transitioning connector portion of the device that may prevent the flow of the one or more therapeutic agents if exposed to forces and/or bending.

FIG. 121 is a drawing of the stem portion of at least one embodiment of the device. The drawing illustrates what can happen to the device when forces are applied to the device and the device bends and/or is contorted, which can cause the reservoir and/or other portions of the device to restrict fluid flow or collapse leading to the fluid path of the one or more therapeutic agent(s) being restricted or blocked.

FIG. 122 is a graph showing stress/strain curves comparison of various materials and multi-lumen tubes that were tested as indicated herein. As an example, when the multi-lumen tube was made out of material 90A, it was shown to perform better than some of the other materials shown in this FIG. 122. See materials on this FIG. 122 that have the largest area under the curve. Various factors were considered including: Ultra Heavy Molecular Weight Polyethylene (C-compression, T-tension), TPU: Thermoplastic Polyurethane (C-compression, T-tension) w/Product Identifiers are shown.

FIG. 123 is a set of schematic drawings of the various multi-lumen configurations that were tested and described herein. The drawings show several different lumen configurations that were tested in real-world conditions to see which of them, if any, would prevent the blockage of the one or more therapeutic devices in the devices shown and described herein. One of the lumen variations commonly referred to as “Double Y” provided the benefit of preventing blockage of the therapeutic agent(s) while providing the desired amount of flexibility/bending moment rigidity, torsional rigidity, maximum flow area and ease of fabrication for the lumen shown and described.

FIG. 124 are drawings showing a sequence of multi-lumen tube deformations that were performed on the various multi-lumen tubes that were tested leading to the eventual folding of the tube in at least some embodiments of the present disclosure described herein.

FIG. 125 are 3-dimensional renderings of the dimensions and orientation of the folded tubes in one embodiment of the present disclosure described herein. It is important to note that there are still some fluid paths on the outside of the crease and the tube is likely not completely blocked off from flow of the one or more therapeutic agents if there were I, H, or Y web cross pieces in the center cross-section of the tube. The webs will prevent a total collapse of the tube walls regardless of the neutral axis orientation. There will always be a fluid path, even when folded a complete 180° upon itself and for a 180°-fold it results in approximately a 325% strain of the material. The internal webbing structure causes increased strain in some parts of the tube but remains well below the material limits.

FIG. 126 is a chart showing the data and results of the various multi-lumen tubes that were tested to determine which performed better than the others.

FIG. 127 is a graph showing test results of the various multi-lumen tubes folded 180°. The test samples are labeled and placed on the graph according to the σ_{U} and ε_{U} properties. The results also indicate the number of lumens for the specific variations that were tested. Only one sample of one lumen tube failed. The stars indicate some of the selected material that performed better than the other materials and configurations that were tested.

FIG. 128 is a graph showing test results of the various multi-lumen tubes that were tested by folding them 180° and twisting them 180°. The various multi-lumen tubes were labeled and placed on the graph according to the σ_{U} and ε_{U} properties. The tubes had a four-lumen layout that were tested consistently and passed. The star indicates one of the materials that performed better than the other materials that were selected and tested.

FIG. 129 is a graph showing microbiological results following quantification of soft and hard tissue samples and metal hardware in a sheep model of long bone open fracture and biofilm implant-related infection. Data show log₁₀ reductions of biofilm burden in the surgical site following one of several treatments. Specifically, moving from left to right: levofloxacin (L) and rifampin (R) were administered systemically (via oral administration) for 10 Days (10D) or 20 Days (20D); vancomycin (V) and tobramycin (T) were administered as direct antibiotic powders in addition to systemic L and R; STIM (Stimulan) beads were loaded with V and T and administered locally in addition to systemic L and R; and the present disclosure described herein (pouch or reservoir) was used to deliver V and T locally for 10 Days (10D) or 20 Days (20D) in addition to systemic L and R. Data indicated that sheep treated with the present disclosure described herein reduced bacteria to a greater degree than those sheep that were not treated with the present disclosure described herein.

FIGS. 130A-J is a set of images showing the device components and subassemblies in at least one embodiment of the present disclosure described herein.—FIG. 130A is the rate-controlling membrane equipped with sacrificial alignment tabs. FIG. 130B is a flat-sheet thermoplastic polyurethane (TPU) with a window for receiving the rate-controlling membrane. FIG. 130C is a flat-sheet TPU cladding for sealing the membrane TPU window interface on the opposing face, with sacrificial alignment tabs. FIG. 130D is a subassembly of FIG. 130A and FIG. 130B after radio frequency (RF) welding and removal of sacrificial alignment pins. FIG. 130E is a subassembly of FIG. 130D and FIG. 130C after the addition of cladding by RF welding and subsequent removal of sacrificial alignment tabs. FIG. 130F is the ¼ inch catheter tube before (top) and after (bottom) a tipping operation designed to receive the deflation assist tri-flute extrusion (bottom right). FIG. 130G is a subassembly of the tipped ¼ in catheter tube and the tri-flute extrusion joined through solvent welding with cyclohexanone. FIG. 130H (top) is the completed flat-sheet subassembly formed subsequent to FIG. 130E by folding, RF, and a trimming operation. FIG. 130H (bottom) is the extrusion subassembly, aligned left and right with the flat-sheet assembly, to show approximate alignment once inserted. FIG. 130I is the extrusion subassembly inserted into the flat-sheet subassembly prior to heat sealing of the neck joint. FIG. 130J is the final device with a heat-sealed neck joint unifying the flat-sheet and extrusion subassemblies.

FIGS. 131A-C is a set of images showing the selection of tooling used in at least one embodiment of the present disclosure described herein. FIG. 131A is a flat-sheet component on alignment pins of an RF welder base prior to welding. FIG. 131B in the ¼ inch catheter tube inserted into a die used in the tipping operation. FIG. 131C in the flat-sheet and extrusion subassemblies aligned on the floating base of a heat-seal press in preparation for forming the neck joint.

FIG. 132 is an illustration showing the current device and the packaging for veterinary sale in at least one embodiment of the present disclosure described herein

FIG. 133 is a diagram showing the neck failure forces of a glue joint (left diagram) and a heat seal weld (right diagram), as used in at least one embodiment of the present disclosure described herein. A heat seal joint produces an ˜3× increase in failure force.

FIG. 134 shows a set of images that demonstrate how earlier versions the device that contained a glue joint (left image) and a heat seal weld (right image) both failed at the neck when enough force was applied to them. While a heat seal joint produced an ˜3× increase in failure force, it still failed under conditions that may occur while being inserted in the animal.

FIG. 135 is an image showing an example of a textured or dimpled surface used for enabling the device to deflate completely in at least one embodiment of the present disclosure described herein.

FIG. 136 is a set of images showing the device in its natural position without any bend, kink, or contortion (image A) and the device being bent at the neck by 180° (image B) during testing as described in the present disclosure.

FIG. 137 is a graph representation of the testing performed on various device deflation technologies in the present disclosure. The deflation assist technologies shown and described herein ensure continued fluid flow even when the device is bent at the neck by 180°. Testing confirmed that the device will not deflate when bent absent use of the deflation assist technologies shown and described herein and in FIG. 137.

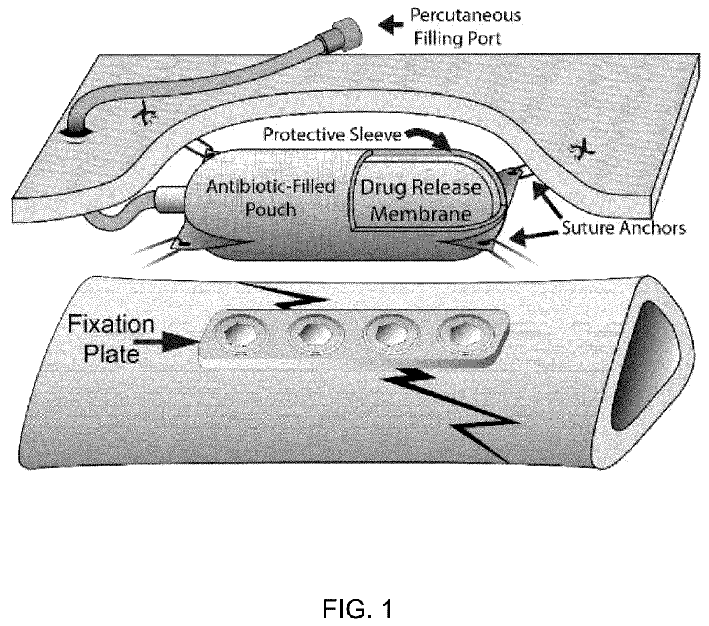

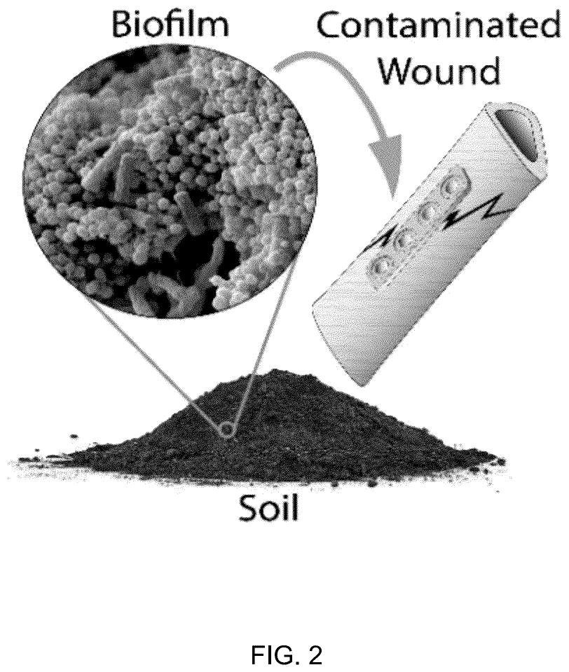

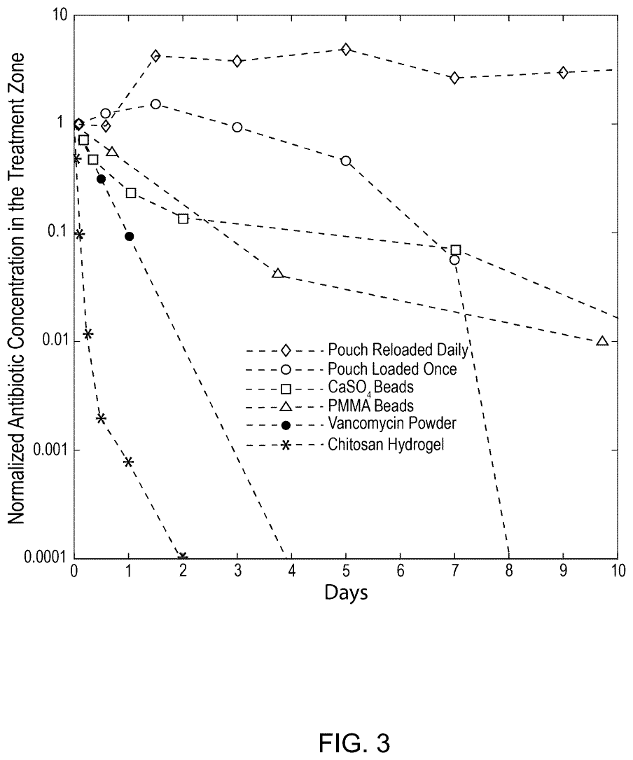

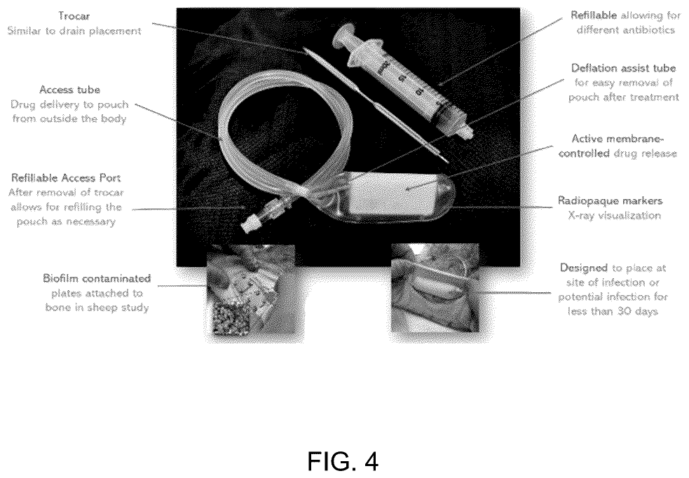

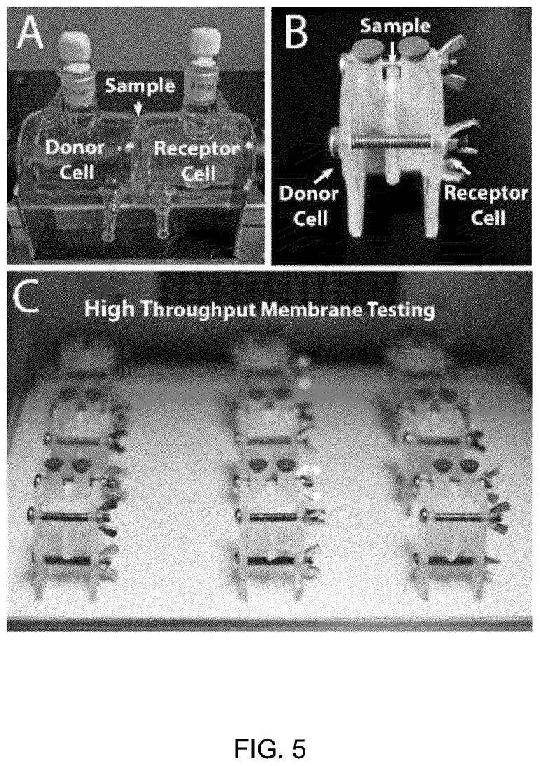

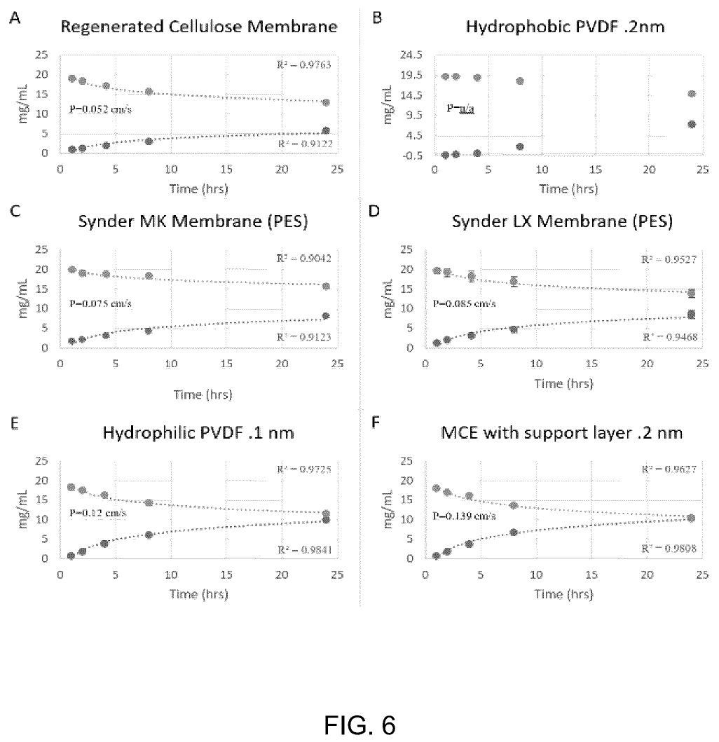

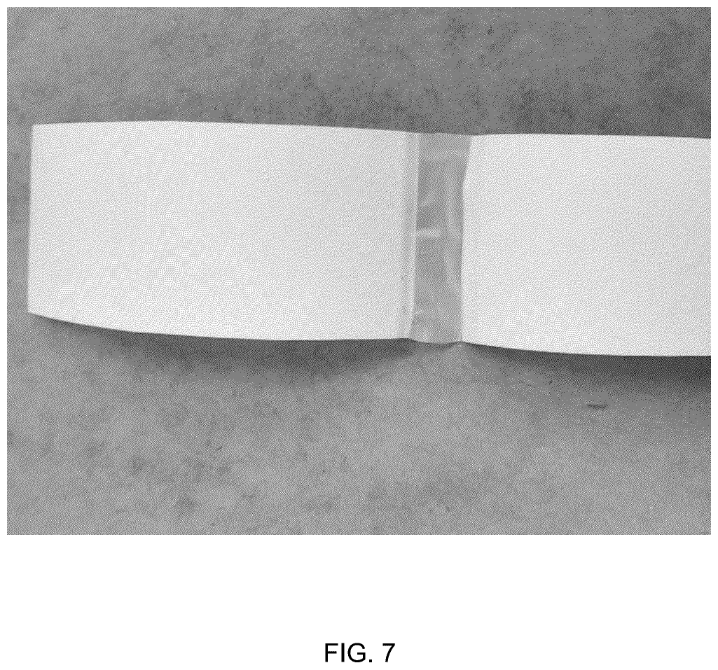

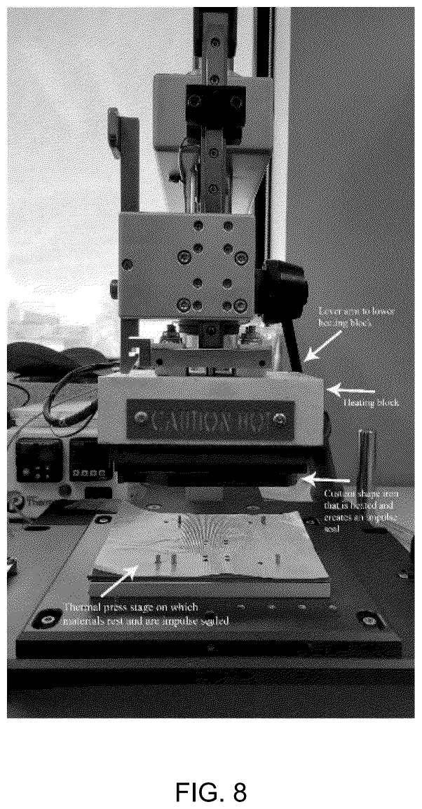

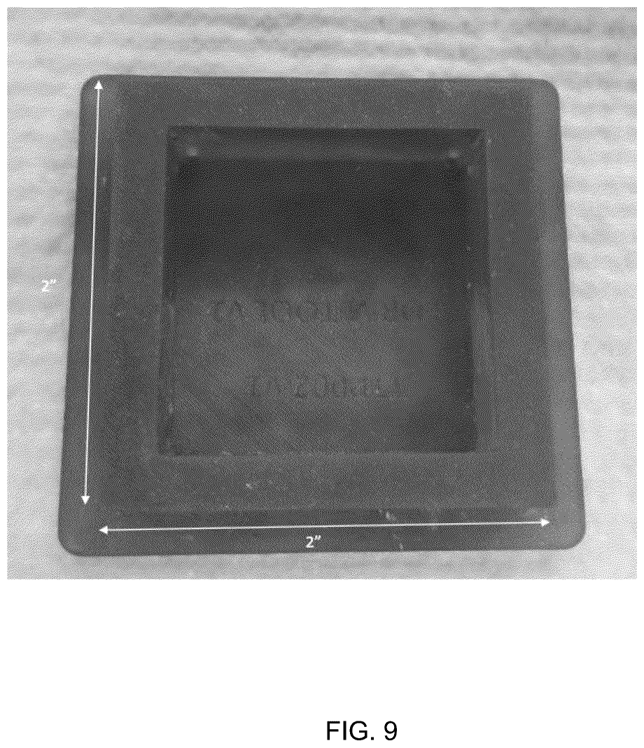

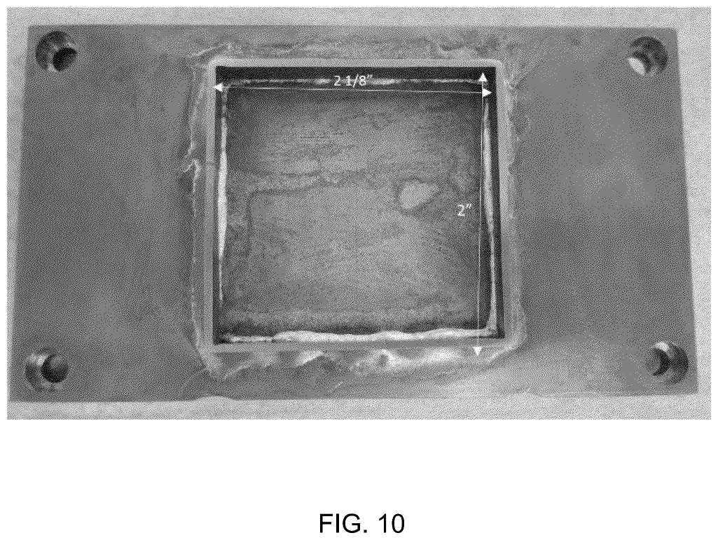

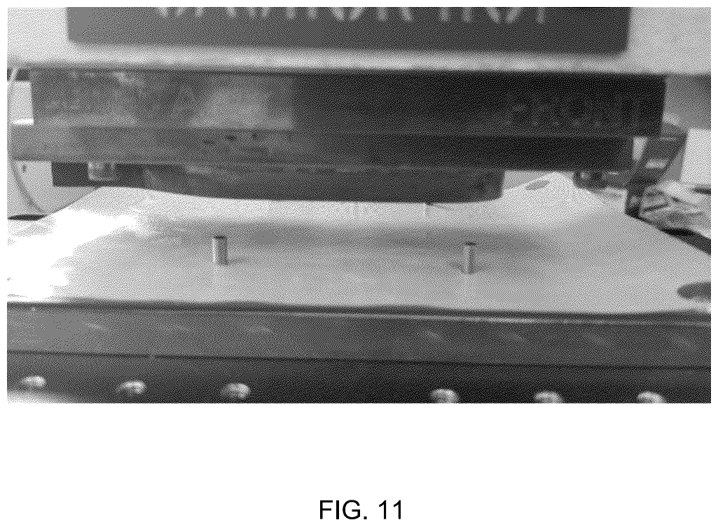

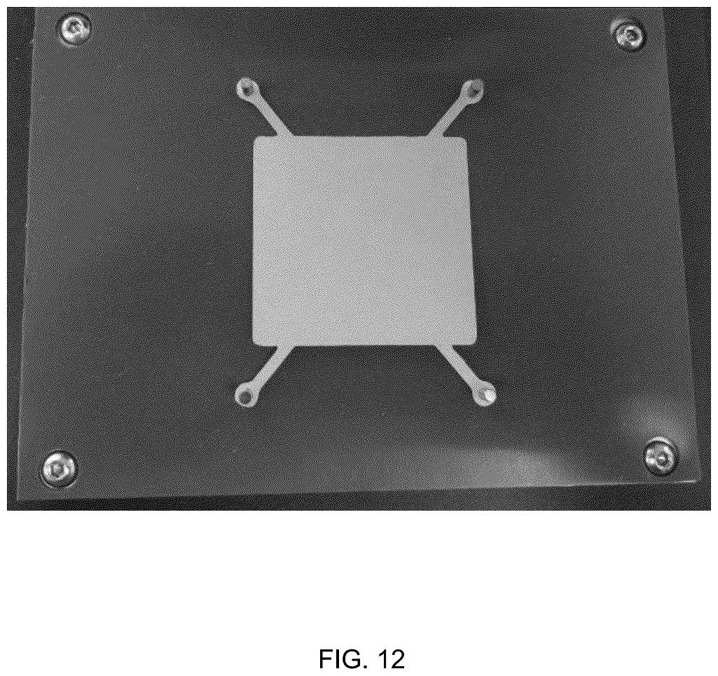

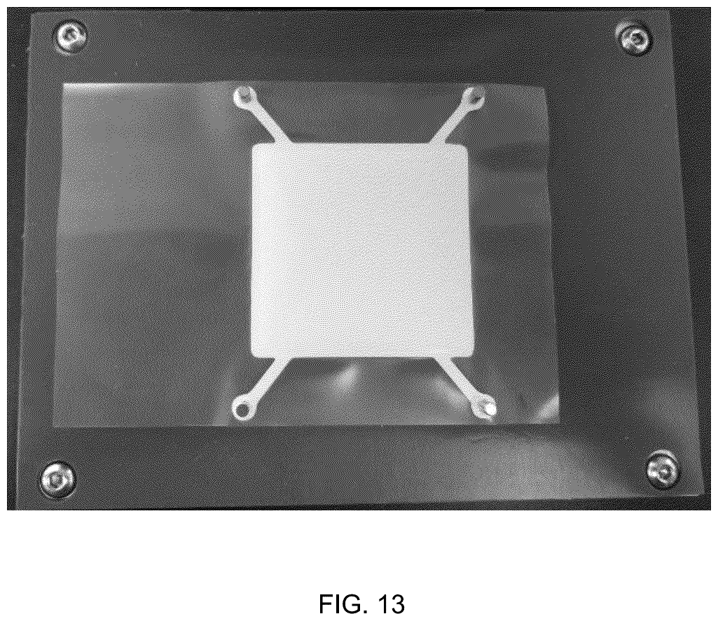

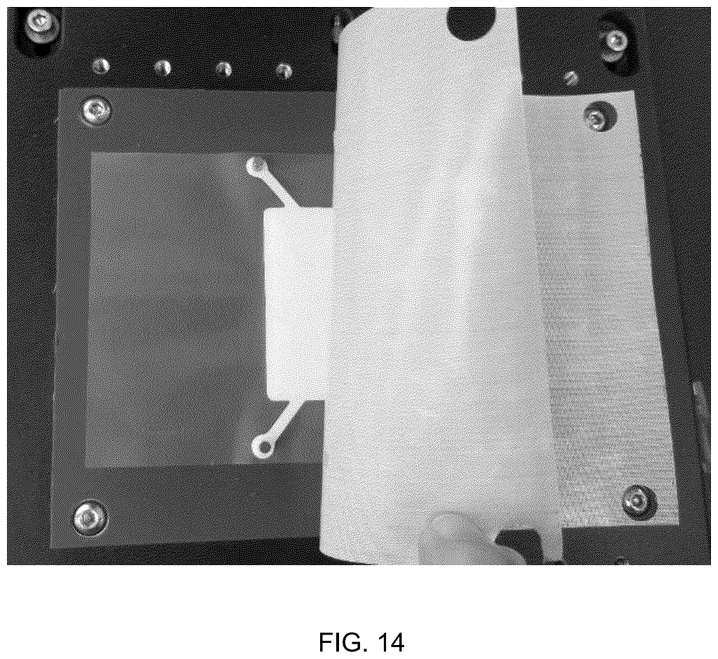

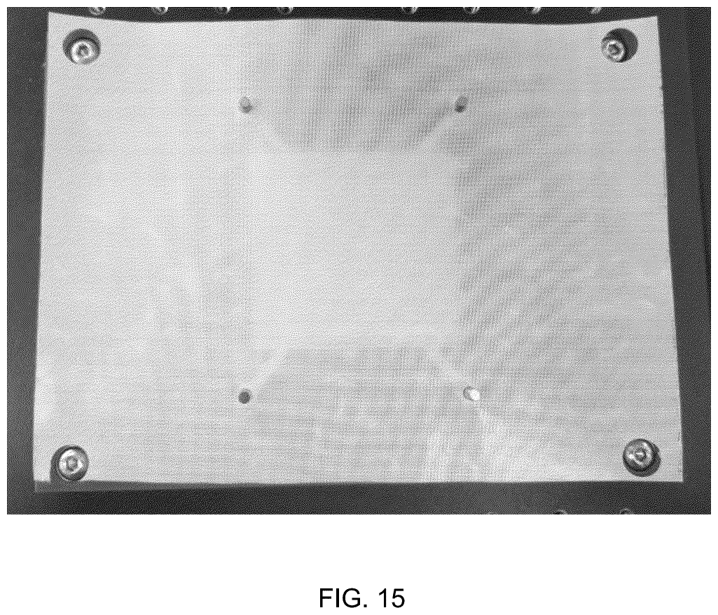

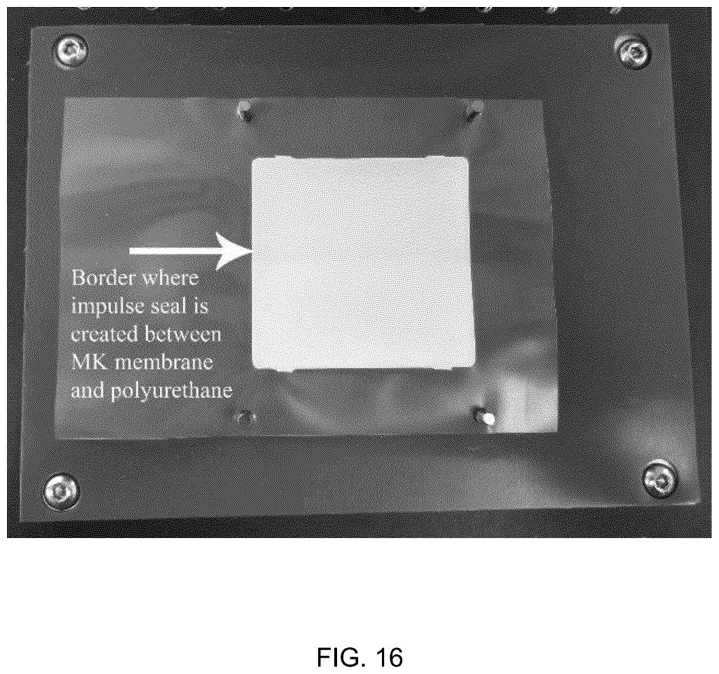

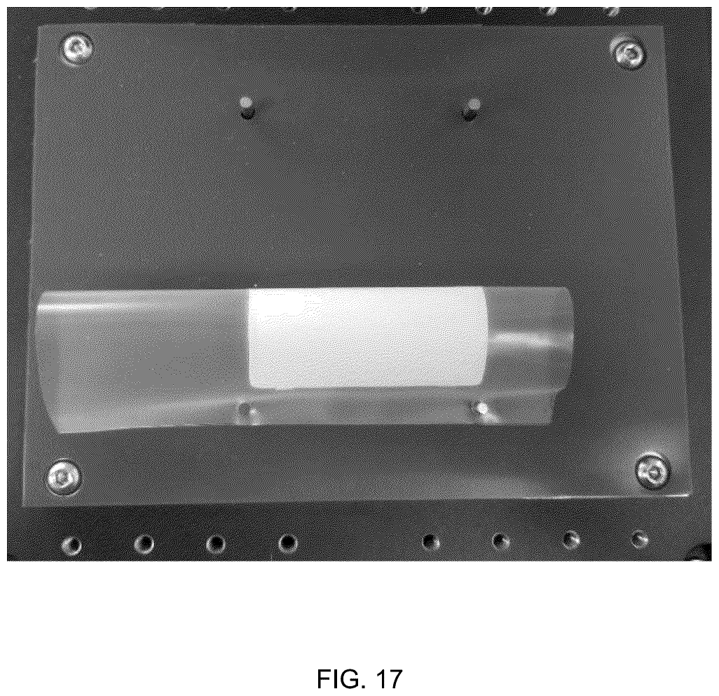

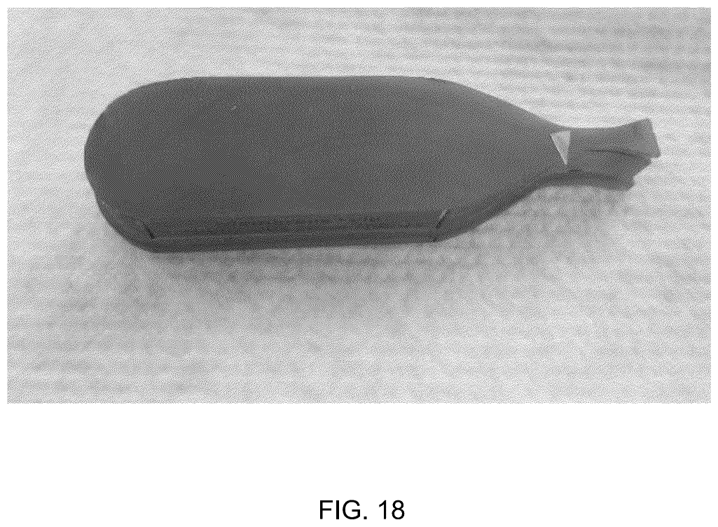

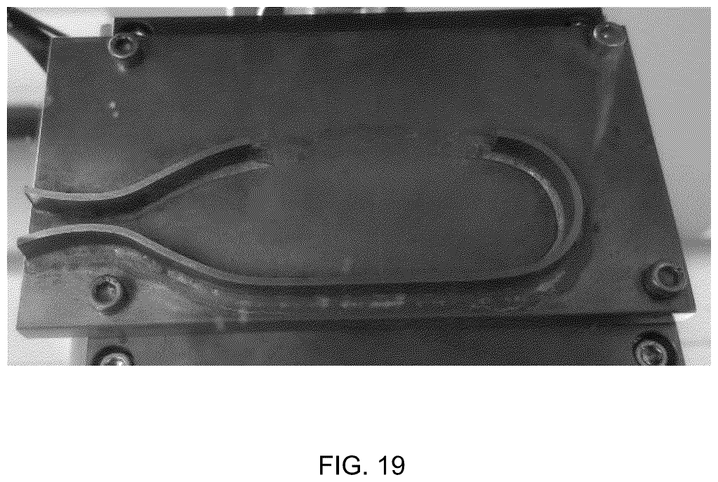

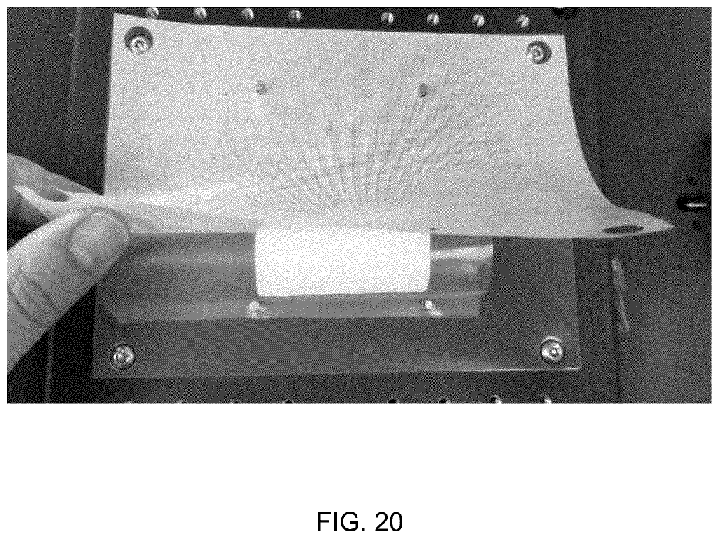

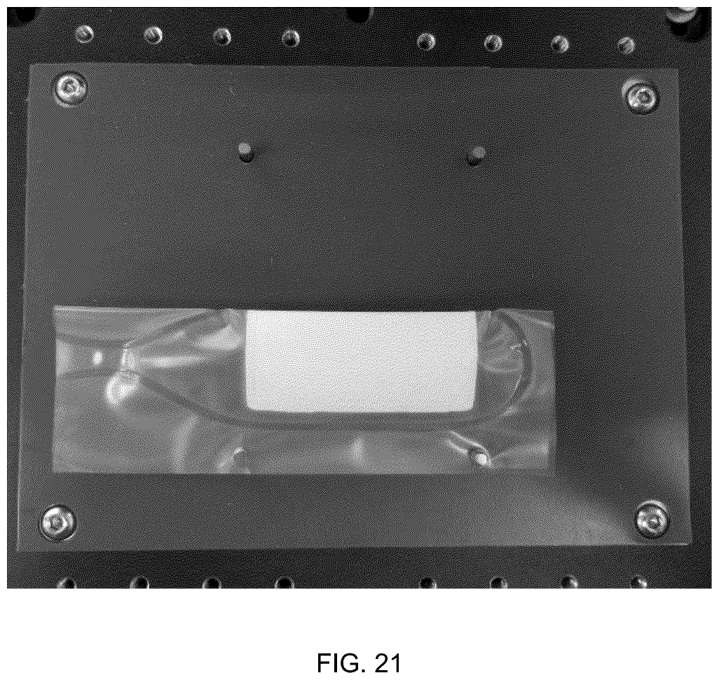

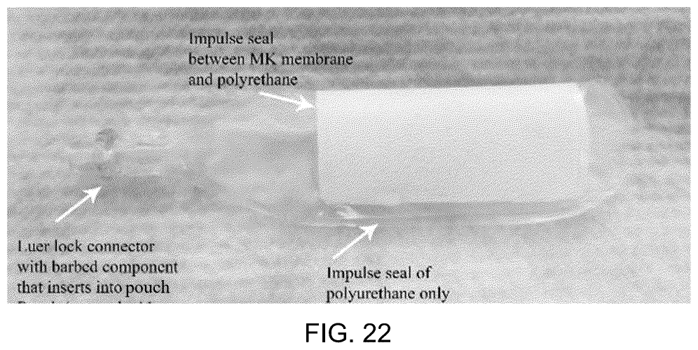

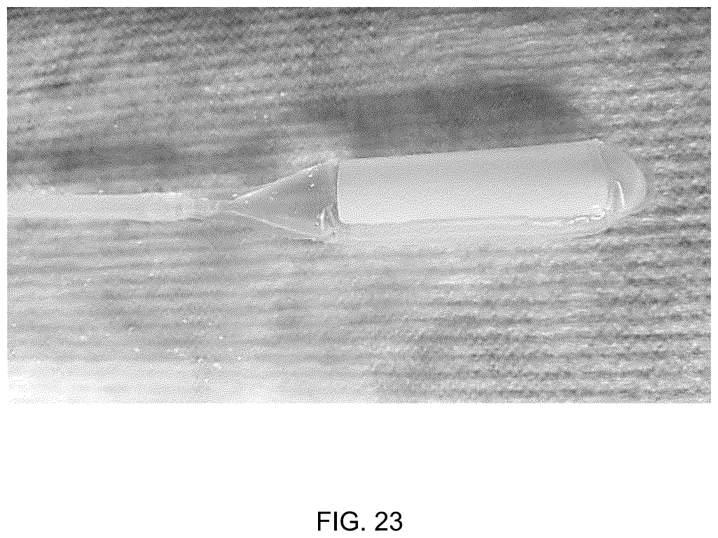

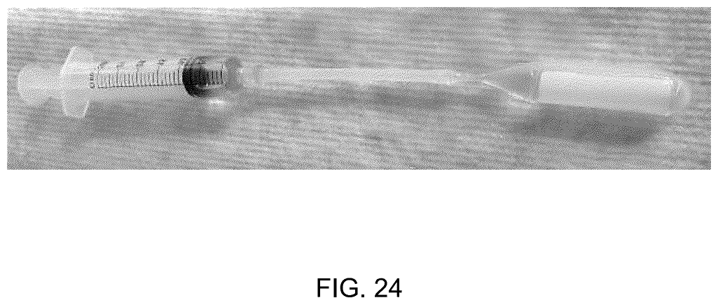

Before any embodiments of the present disclosure are explained in detail, it is to be understood that the invention is not limited in its application to the details of construction and the arrangement of components outlined in the following description or illustrated in the following drawings. The invention is capable of other embodiments and of being practiced or of being carried out in various ways. Also, it is to be understood that the phraseology and terminology used herein should be given their broadest possible interpretation and are for description and should not be regarded as in any way limiting the invention(s) described herein.

### DETAILED DESCRIPTION

Preliminary in vitro tests, as shown and described herein, have shown efficacy using the devices disclosed herein by eradicating both biofilm and planktonic bacteria in the presence of the antimicrobial-releasing device. For example, under flow conditions consistent with lymphatic clearance rates of viable tissues (fluid exchange rate of approximately 14%/hr) in brain heart infusion broth, the device (filled with 15 mL of PBS that contained 70 mg/mL fosfomycin, 25 mg/mL gentamicin and 2 mg/mL rifampin) was able to fully eradicate 10⁹ colony forming units (CFU) of methicillin-resistant Staphylococcus aureus (MRSA) within a 24 hr period. In the presence of serum, the same antibiotic combination in the device was able to reduce MRSA biofilms by more than 6 log₁₀ units in 24 hr.

In vivo testing in a sheep model of long bone fracture, wherein the device was placed subdermally, yet directly over an implant site that contained 10⁹ CFU of MRSA in biofilms, showed the ability of the devices shown and described herein to treat and prevent biofilm implant-related infection. In other in vivo tests using the same sheep model, the device successfully treated infection caused by Staphylococcus aureus, Pseudomonas aeruginosa, or both. In other in vivo tests performed under Good Laboratory Practices using the same sheep model without bacterial inocula, the device showed no signs of tissue toxicity and serum antibiotic levels were within or below typical ranges of antibiotics that are administered systemically. Data strongly supports that the device is safe and effective.

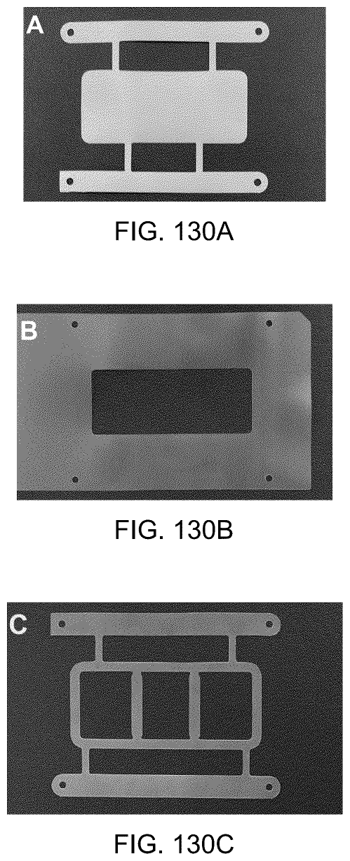

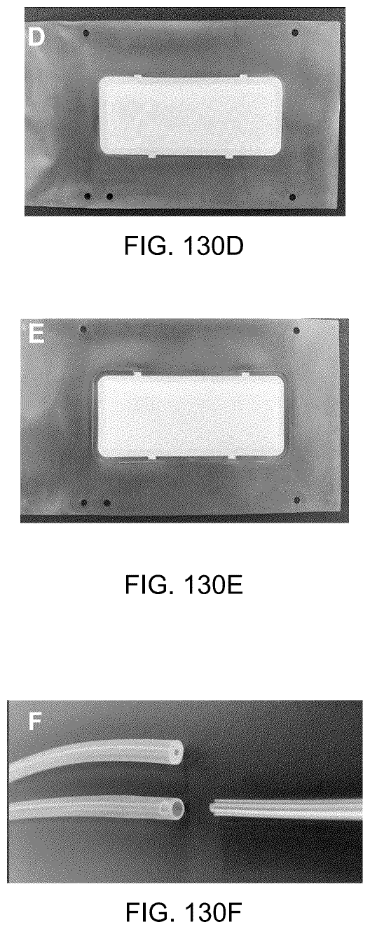

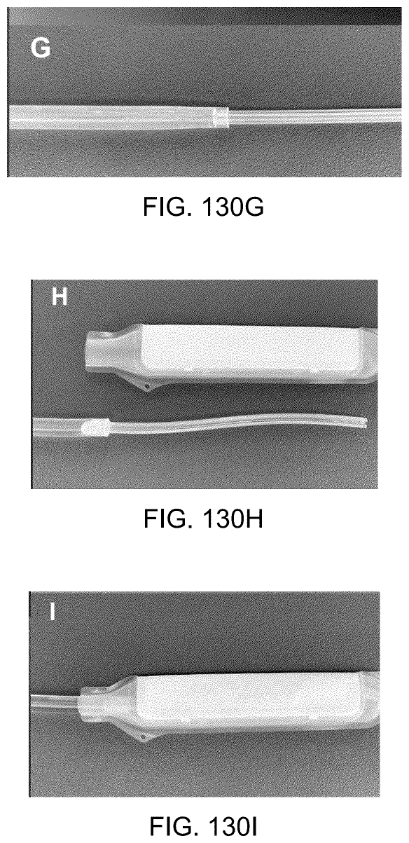

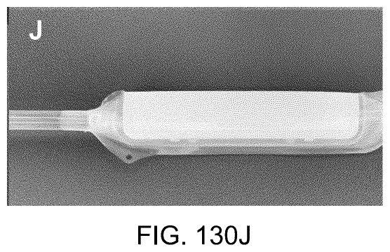

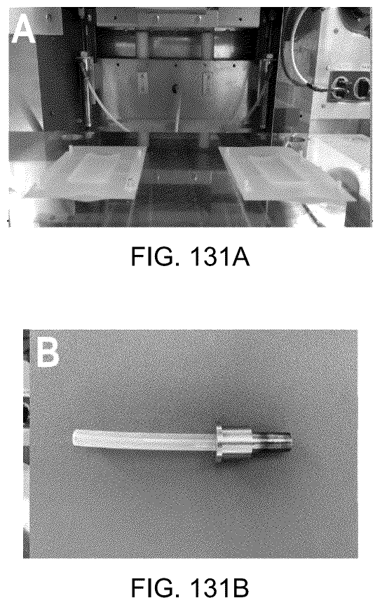

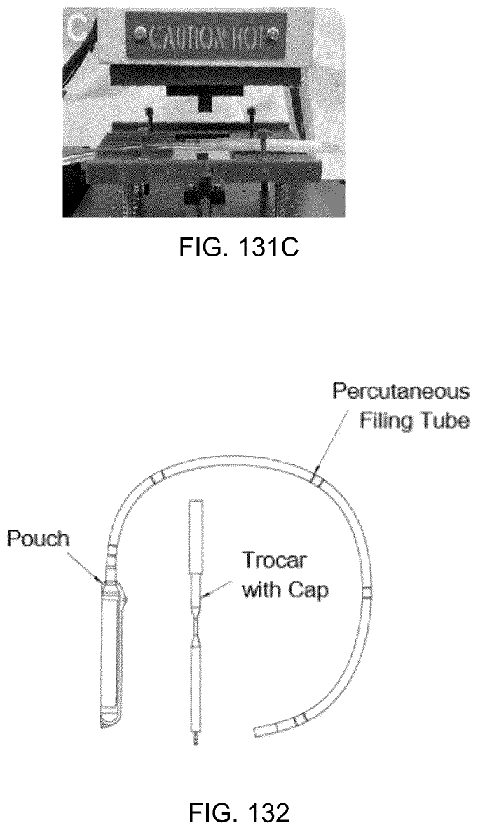

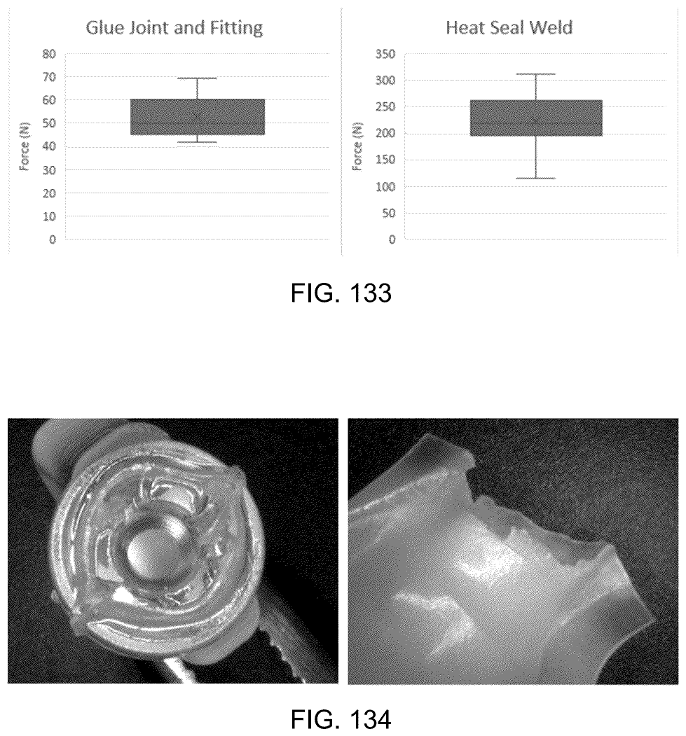

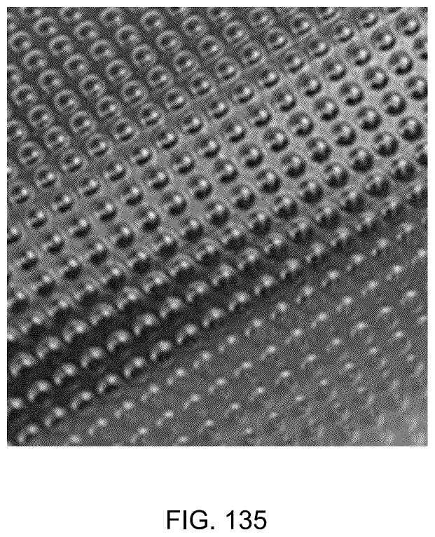

In one embodiment of the present disclosure, a device is comprised of the rate-controlling composite membrane, which is radio frequency (RF) welded to the flat sheet polyurethane (PU) and RF welded with PU cladding to form the rate-controlling treatment reservoir (see FIG. 130A-E). In at least one embodiment, a trimming die aids in shaping the treatment reservoir and forms suture anchor holes. The thermoplastic polyurethane (TPU) percutaneous refilling subcomponent is tipped, the extrusion subassembly inserted, and solvent welded in place with the aid of custom manufacturing fixtures (see FIG. 130G-H). In at least one embodiment, the extrusion subassembly is then inserted into the treatment reservoir, the neck of the flat sheet PU is brought over the refilling tube, and the neck joint is thermally welded together. When complete, all materials and subassemblies form a contiguous, fully integrated pouch (see FIG. 130I-J).

The rate-controlling membrane broadly describes membranes which can be used with any of the embodiments of the present disclosure, rather than a narrower term “semi-permeable membrane”. Nearly all membranes fit broadly into one of two classes: size exclusion membranes and affinity membranes. Size exclusion membranes are semi-permeable membranes which use physical pores to selectively pass solutes. These include: ultrafiltration membranes, microfiltration membranes, nanofiltration membranes, and dialysis membranes. In at least some embodiments, passage of solutes through these membranes is limited by the diffusion rate of the solute through the limited number of tortuous membrane pores. Affinity membranes, on the other hand, use molecular affinity interactions between the solute and the membrane components or functional groups to slow down the solute diffusion rate within the membrane. Affinity membranes include hydrogels, polymer systems, and functionalized polymer systems. If selected carefully, any number of these membranes or membrane types might be used in the device to achieve the target therapeutic delivery profile.

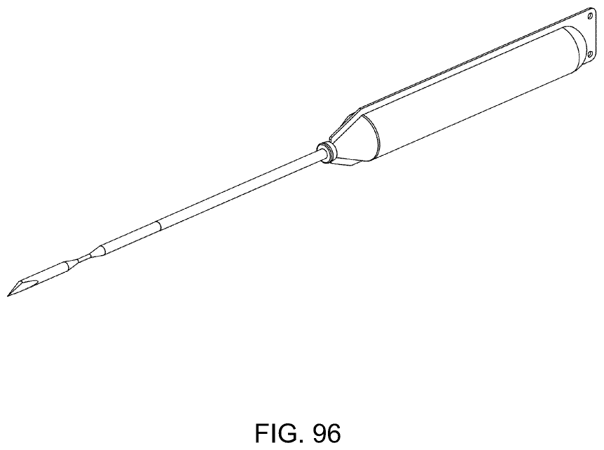

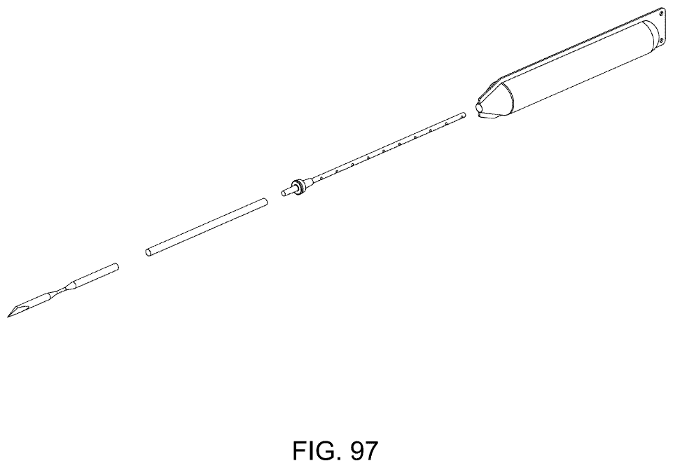

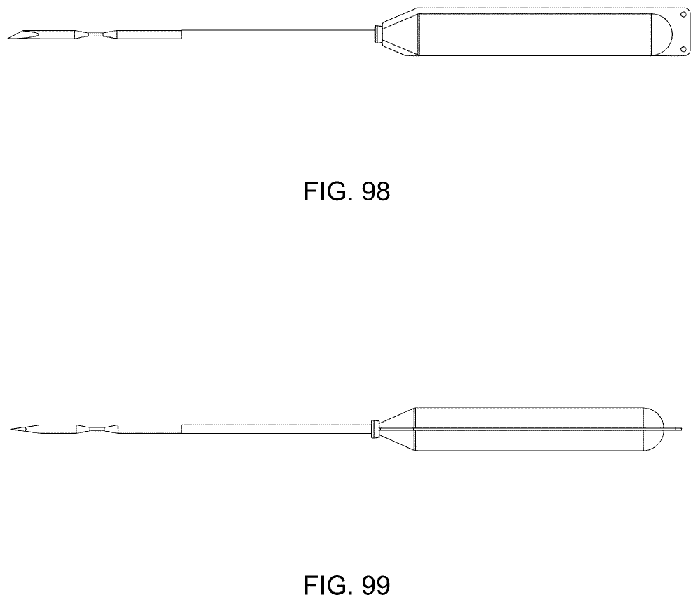

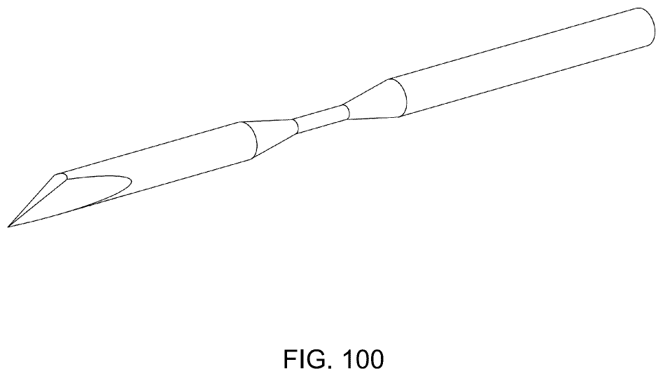

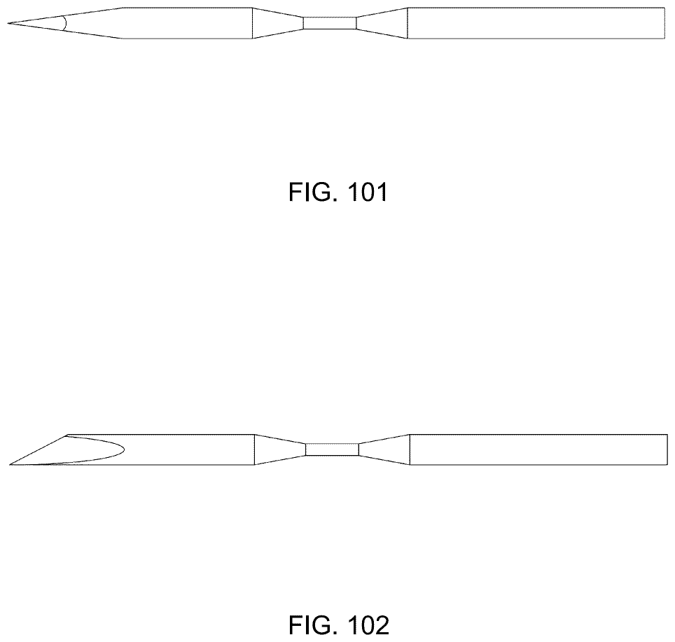

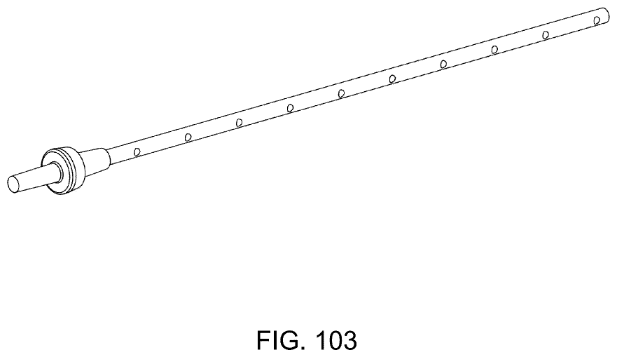

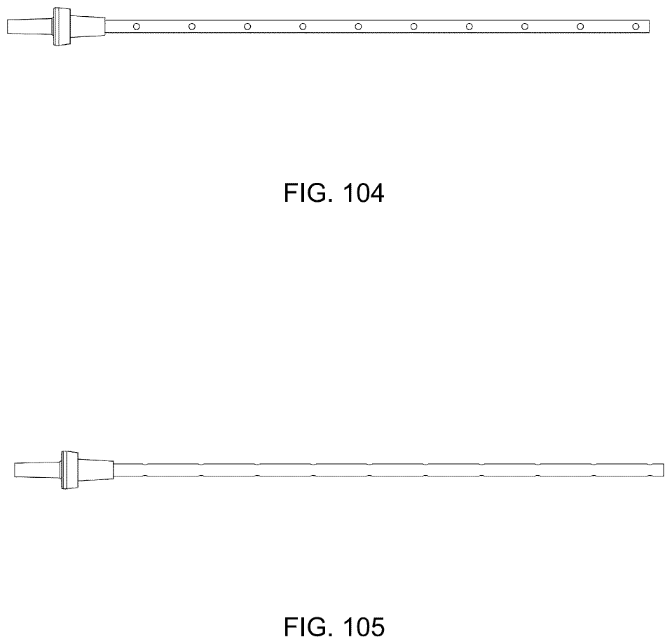

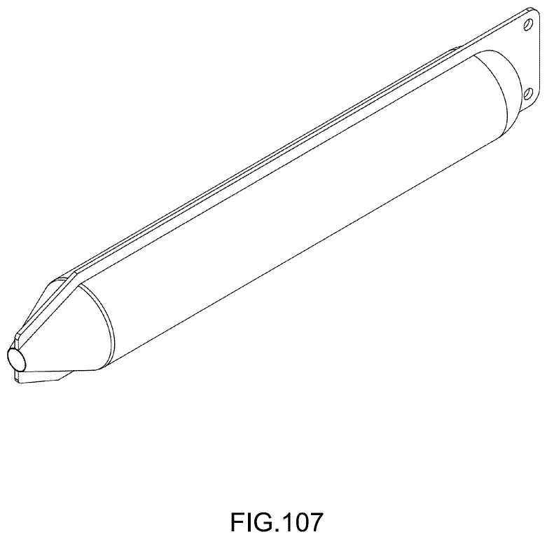

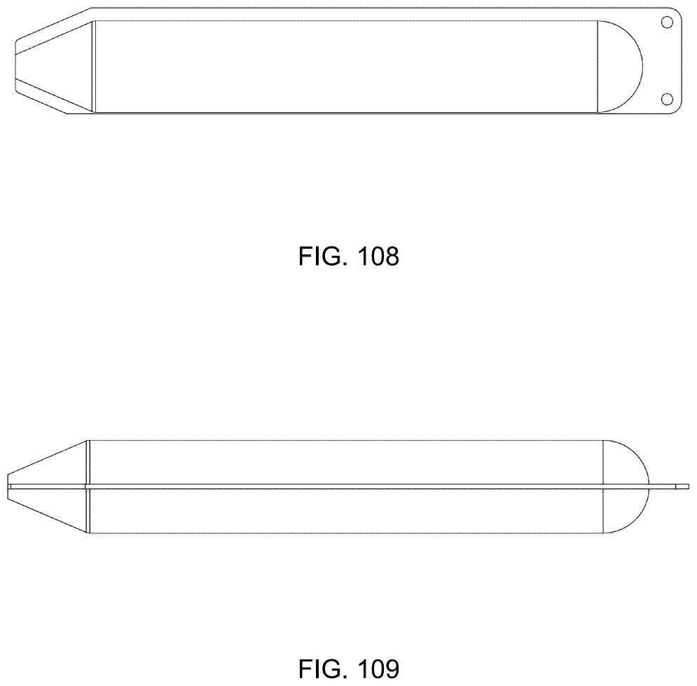

FIGS. 96-109 illustrate various embodiments and components of the device disclosed herein that allow for the subcutaneous delivery of one or more therapeutic agents (e.g., antibiotics, antimicrobials, anti-tumor agents, steroids, analgesics, anti-inflammatory, etc.) to a wound site, surgical site, or body cavity of an animal in need thereof. In one embodiment, the reservoir is placed within a wound/surgical site to deliver an antimicrobial agent within the wound/surgical site to prevent or treat infections. Examples of such wounds/surgical sites include implants/implant sites, abscesses, chronically infected wounds, and other localized infections. In one example, the device shown and described herein may be placed within a wound/surgical site after an open fracture of a bone within one of an animal's extremities (e.g., reduced with a fixation plate) to provide high doses of antimicrobial agents over an extended duration to prevent and treat infection.

Referring to FIGS. 96-109, the reservoir includes a port that has a stem. The port is in fluid communication with the reservoir such that a liquid antimicrobial agent may be introduced into the reservoir.

A rate-controlling membrane is disposed in the reservoir that allows the one or more therapeutic agents to be delivered to the animal at a controlled rate and the reservoir selectively communicates therapeutic agents (e.g., antimicrobials) with the immediate and local surrounding environment. That is, the membrane facilitates a controlled release of one or more therapeutic agents. The rate-controlling membrane disposed in the reservoir allows the one or more therapeutic agents to be delivered to the animal at a controlled rate.

In another embodiment, a therapeutic delivery device comprising (1) an at least semi-rigid body defining at least one internal reservoir for holding one or more therapeutic agents that are to be delivered to an animal, (2) a porous rate-controlling membrane forming the internal reservoir or located in the internal reservoir that controls the rate of release of the therapeutic agent to the animal and (3) a percutaneous refilling tube in fluid communication with the internal reservoir where at least a portion of the semi-rigid body is comprised of the porous rate-controlling membrane or at least the edges of the porous rate-controlling membrane are sealed and/or cladded with at least a portion of the semi-rigid body to form a contiguous, fully integrated therapeutic device.

In at least one embodiment of the present disclosure, the tube of the percutaneous refilling tube is approximately 0.25 in OD and 0.062 ID and comprises of a relatively transparent Pellethane tube. It should be appreciated that other tubes could be used for the percutaneous refilling tube that could be made of a material that is similar material of semi rigid body of the device and could be used for heat bonding. In at least one embodiment, the tube of the percutaneous refilling tube would have a small ID to reduce the amount of dead volume (e.g., no therapeutic agent) in the reservoir or the device). In at least one embodiment, the tube is made of a transparent material so the user can observe the flow of the one or more therapeutic agents in and out of the devices of the present disclosure. It should be appreciated that the percutaneous refilling tube can be made of any polymeric cannulated extrusion.

In at least one embodiment of the present disclosure, the semi-rigid body is made up of one or more polyurethanes and/or polyurethane-like biocompatible materials. It should be appreciated that the semi-rigid body can be comprised of any polymer that is appropriate for medical uses.

In at least one embodiment of the present disclosure, the membrane is comprised of PES (Polyether sulfone). It should be appreciated that the membrane could be made of any material that can act as a membrane that will control the flow of the one or more therapeutic agents. In at least one embodiment of the present disclosure the membrane can be a paper-like material that controls the flow of the one or more therapeutic agents.

It should be appreciated that the membrane can be made of various other materials that have the appropriate diffusion characteristics and the membrane material has the appropriate bond strength with the other materials used in the device.

In another embodiment of the present disclosure, the device also includes a fluted extrusion in fluid communication with at least one internal reservoir.

The fluted extrusion of the device solves the problem of not being able to remove some to all of the one or more therapeutic agents from the device even if the reservoir of the device is twisted, bent or otherwise impeded while it is inside the animal that is being treated.

In at least one embodiment of the present disclosure, the fluted extrusion is made out of the same material as the tubing and is solvent or heat-bonded to the tubing.

In one aspect of the present disclosure, the fluted extrusion has one to ten channels or tubes.

It should be appreciated that the fluted extrusion could be comprised of different materials and the tubing of the fluted extrusion can be glued or solvent welded together.

In another embodiment, a device for delivering one or more therapeutic agents subcutaneously to an animal in need thereof is provided. The device comprises (1) a port for receiving one or more therapeutic agents that is to be delivered to an animal subcutaneously, the port having a stem and (2) a reservoir in fluid communication with the stem and comprised of at least two areas, the first area being comprised of a more rigid elastic material and the second arca being comprised of a less rigid elastic material and (3) a rate-controlling porous or non-porous membrane disposed in the reservoir that allows the one or more therapeutic agents to be delivered to the animal at a controlled rate. The reservoir is capable of being inserted subcutaneously into an animal in need of the one or more therapeutic agents and filled or refilled to contain the therapeutic agents that are to be delivered to the animal subcutaneously while at least the reservoir of the device is inserted subcutaneously in the animal in need of one or more therapeutic agents.

In at least one embodiment, the less rigid elastic material is comprised of a thermoplastic polyurethane PZMED film sold by Polyzen Inc. It should be appreciated that a variety of thicknesses and durometers can be used. In at least one embodiment, we have used an 0.008″ thick 80A PZMED material is used for the less rigid elastic material.

In at least one embodiment of the present disclosure, the less ridged clastic material is comprised of a thermoplastic polyurethane film created by Polyzen Inc. In at least one embodiment of the present disclosure, the PZMED film by Polyzen Inc. is used. It should be appreciated that a variety of thicknesses and durometers. We have used many of them in development. The current material we are using is an 0.008″ thick 80A PZMED material.

At least the reservoir portion of the device is capable of being inserted subcutaneously into the body of the animal in need of one or more therapeutic agents and the one or more therapeutic agents can be removed from the reservoir of the device or filled or refilled into the reservoir of the device without the device being removed from the body of the animal in need of the one or more therapeutic agents.

In at least one embodiment of the present disclosure, the rate controlling membrane is comprised of the Synder MK ultra filtration membrane. Various other rate-controlling membranes that could be used include, but are not limited to, PES membranes XT, VT, MT, ST, SM, MQ, LY, LV or LX. For each rate-controlling membrane, testing for diffusion rate would need to be performed to verify functionality, performance, and safety. Other membranes sold by various vendors, including but not limited to Pall Corporation, Cytiva, Merck Millipore, and membranes PZ, PY, PX, V3-7, or BN, BY, BX, A6 sold or created by Synder could be used. All of these materials would need to be assessed for compatibility. Other membrane suppliers could be used, but all would need to be assessed for compatibility. These suppliers include, etc.

In another embodiment, a device for delivering one or more therapeutic agents subcutaneously to an animal in need thereof is provided. The device comprises (1) a port for receiving one or more therapeutic agents that is to be delivered to an animal subcutaneously, the port having a stem; (2) a reservoir in fluid communication with the stem and comprised of a less compliant elastic material abutting a more compliant elastic material at least at the edges of the reservoir, the reservoir being capable of being inserted subcutaneously into an animal in need of the one or more therapeutic agents and filled or refilled to contain the therapeutic agents that are to be delivered to the animal in need thereof; and (3) a rate-controlling porous or non-porous membrane disposed in the reservoir that allows the one or more therapeutic agents to be delivered to the animal at a controlled rate where at least the reservoir portion of the device is capable of being inserted subcutaneously into the body of the animal in need of one or more therapeutic agents and the one or more therapeutic agents can be removed from the reservoir of the device or filled or refilled into the reservoir of the device without the device being removed from the body of the animal in need of the one or more therapeutic agents.

In yet another embodiment, a device for delivering one or more therapeutic agents subcutaneously to an animal in need thereof is provided. The device comprises (1) a port for receiving one or more therapeutic agents that is to be delivered to an animal subcutaneously, the port having a stem; and (2) a reservoir that is comprised of a compliant elastic material and a rate-controlling membrane that allows the one or more therapeutic agents to be delivered to the animal at a controlled rate, wherein the rate-controlling membrane is disposed in at least the center area of the reservoir and a compliant elastic material that is disposed in at least the end of the reservoir and rate-controlling membrane and the compliant elastic material being attached to each other to form at least a semi-continuous layer and the reservoir being capable of being inflated to contain one or more therapeutic agents and deflated to be substantially flat to remove from the animal in need thereof or to receive or refill one or more therapeutic agents that are to be delivered to the animal in need thereof, the reservoir being in fluid communication with the stem of the device.

In another aspect of at least one embodiment, at least the reservoir portion of the device is capable of being inserted subcutaneously into the body of the animal in need of one or more therapeutic agents and the one or more therapeutic agents can be removed from the reservoir of the device or filled or refilled into the reservoir of the device without the device being removed from the body of the animal in need of the one or more therapeutic agents.

In another aspect of at least one embodiment, the reservoir is further comprised of a reinforcement or composite membrane located between the more rigid elastic material and the less rigid elastic material of the reservoir.

In another aspect of at least one embodiment, the reinforcement or composite membrane is cladded on both sides of the reinforcement or composite membrane with the more rigid elastic material or the less rigid elastic material and the more rigid material and the less rigid material overlap at least a portion of the reinforcement or composite membrane and create a heat seal with the reinforcement membrane in the reservoir.

It should be appreciated that in at least one embodiment of the present invention, the cladding is a secondary seal meant to reinforce the bond between the membrane and the polyurethane reservoir. The cladding can be comprised of a die-cut ring (which can be made from the same or a different material as the reservoir).

In at least one embodiment, the cladding is placed on the opposite side of the first heat weld. The inner half of the ring lies overtop the membrane, and the outer half of the ring lies overtop the reservoir material.

In at least one embodiment of the present disclosure, the cladding is heat-sealed or solvent-welded to the membrane and reservoir. In another aspect of at least one embodiment of the present disclosure, the original membrane to reservoir weld and the cladding weld, the membrane window is sealed on both sides to increase strength.

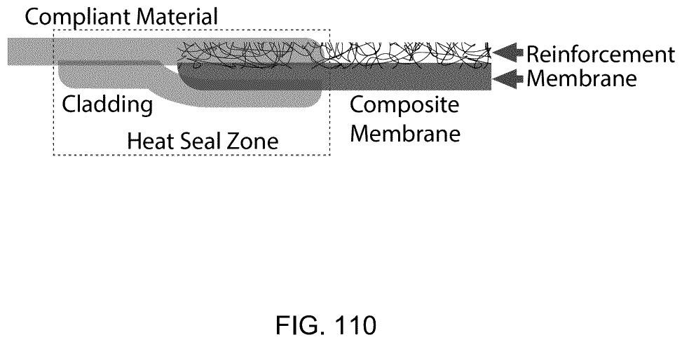

One of the benefits of the cladding described herein is that it seals the outside edge of the membrane and prevents the liquid inside the reservoir from bypassing the membrane of the device. The cladding improves the design because the membrane is two layers of materials bonded together and the primary heat seal alone does not provide a complete and durable seal to the membrane or reinforcement layer of the membrane. In at least one embodiment, the bypassing comes through the outside edge of the membrane. Sealing of the device through cladding can be accomplished through a variety of way, including but not limited to with glue to seal the outside edge of the membrane (See FIG. 110).

In at least one aspect of at least one embodiment, the more rigid elastic material and the less rigid elastic material sufficiently overlap the reinforcement or composite membrane and create a heat sealed joint of the more rigid elastic material, the less rigid elastic material and the reinforcement or composite membrane at least at the outer edges of the reservoir.

In at least one aspect of at least one embodiment, at least a substantial portion of both ends of the reservoir is comprised of the more rigid elastic material, and at least a substantial portion of the middle of the reservoir is comprised of the less rigid elastic material.

In at least one aspect of at least one embodiment, the device does not have a sleeve covering the reservoir, the reservoir is tapered from at least the reservoir to the stem and the largest diameter of the device when the reservoir does not contain any therapeutic agents is the diameter of the port or the stem allowing for easy removal of the device from the animal in need of one or more therapeutic agents.

In at least one aspect of at least one embodiment, the device further comprises one or more tabs or holes near the edges or outside the reservoir that can be used to fasten the device subcutaneously to the animal in need of the one or more therapeutic agents.

In at least one aspect of at least one embodiment, the rate-controlling membrane has pores and the device further comprises one or more chemical stabilizers or plasticizers to keep the pores of the rate-controlling membrane open

In at least one aspect of at least one embodiment, the chemical stabilizer is, by way of example and not limitation, glycerol.

In yet another embodiment, the body has an end of the outer wall that is opposite the port heat-sealed to at least partially enclose the reservoir. However, other methods of sealing the body to form an internal reservoir may be utilized. In another embodiment, the membrane is a semi-permeable size exclusion membrane with a molecular cut off of approximately 0.1-0.5 kDaltons (e.g., based on the molecular weights of cefepime, levofloxacin, fosfomycin, gentamicin, or rifampin). However, this and the other embodiments disclosed herein are merely exemplary. The molecular weight cut off of the semi-permeable membrane may be selected/customized to function with other therapeutic agents. For example, membranes in the nano- and ultra-filtration ranges with pore sizes from approximately 1-100 nm and molecular cutoffs from 1-14 kDa may be used. The semi-permeable membrane, in this example, is configured to enable delivery of the antimicrobial/therapeutic agent to the surrounding tissue according to an elution profile as defined by characteristics of the semi-permeable membrane.

For example, and not by way of limitation, the semi-permeable membrane may be configured to regulate molecular mobility and slow down the diffusion of the liquid antimicrobial agent through the semipermeable membrane. In other examples, the membrane may create a steady-state elution profile or a variable rate elution profile, among others. In any case, the elution profile is set such that a high concentration of the antimicrobial agent is maintained within the surgical site for a predetermined amount of time to prevent local infection. In addition, the device can be modular such that rate of release (based on membrane or other material selection) can be rapid, given as a bolus dose, which may be beneficial to eradicate biofilms quickly, or throttled to deliver lower doses over a longer period of time, which may be of interest for an alternate indication such as pain management. Other factors that may be utilized to control elution profiles include the density of pores on the semi-permeable membrane and the thickness of the semi-permeable membrane.

In another embodiment, the stem is a non-permeable tube interconnecting the injection cap and the body. As such, the therapeutic agent is delivered from the injection cap to the body through the tube. In other embodiments, however, the stem may be at least partially constructed from a membrane (e.g., a similar membrane as described above) such that the therapeutic agent is delivered to a greater volume within the wound site. For example, in this configuration, the therapeutic agent would further be delivered directly to the areas surrounding the percutaneous incision to prevent surgical site infection.

In yet another embodiment, the stem may have holes in it that facilitate filling and emptying of the reservoir.

In yet another embodiment, the stem may be variable based on the application. For example, the length of the stem may be varied, the type of connection may be varied (e.g. needled or needleless connection), and/or can be made of various materials (e.g., permeable or non-permeable materials). In addition, the device may include multiple stems to, for example, facilitate the introduction of different therapeutic agents or to form an inlet to fill the reservoir and a separate outlet to empty the reservoir.

In yet another embodiment, the port and, more specifically, the stem, may extend through the reservoir of the body to provide structural support to the body, aid in maintaining a more uniform body profile (e.g., when the reservoir is empty), and to facilitate more even filling of the reservoir. In this embodiment, at least a portion of the stem (e.g., the portion lying within the reservoir) includes apertures, perforations, permeable membrane portions, or other means for fluid communication between the stem and the reservoir.

In yet another embodiment, the device further comprises a multi-lumen tube that is in fluid communication with at least the stem of the device.

In yet another embodiment, the reservoir is further comprised of a reinforcement or composite membrane located between the more rigid elastic material and the less rigid elastic material of the reservoir and the reinforcement or composite membrane is cladded on both sides of the reinforcement or composite membrane with the compliant elastic material and the reinforcement or composite membrane and the compliant elastic material overlap at least at portions of the reservoir and create a heat seal for the reservoir.

In yet another embodiment, the device further comprises a multi-lumen tube that is located at least at the stem of the device.

The therapeutic delivery device may vary in size. In one embodiment, the body is approximately 10 cm long. However, all aspects of the device may be tailored for specific uses and specific placements within the anatomy. For example, the size of the body or the length of the stem may be varied (e.g., made longer so the device may be placed deeper into tissue). In addition, an introducer device may be used to aid in deployment of the device in applications when a separate surgery is not being performed (e.g., in the case of treatment as opposed to prophylaxis) such as treatment of osteomyelitis or infections associated with previously implanted devices.

The therapeutic delivery device has been tested for in vitro efficacy in treating biofilms. In one test, the device was filled with an antibiotic solution and placed in test tubes that contained either 10⁸ CFU/mL or well-established biofilms of MRSA. Fresh bacteria and solution were added to the test tubes daily for 10 days and quantified each day. With n=6 repeats, it was shown that planktonic and biofilm bacteria were eradicated completely.

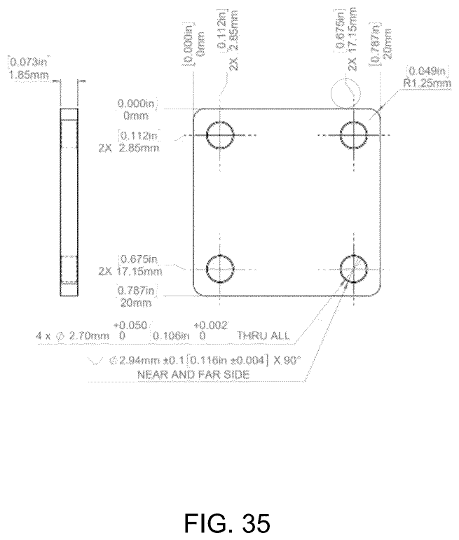

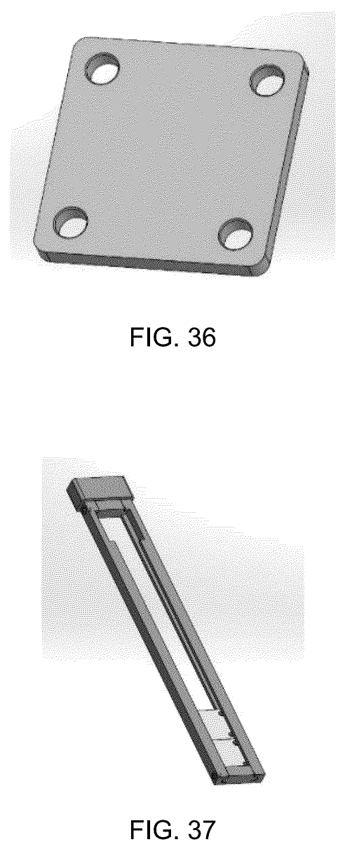

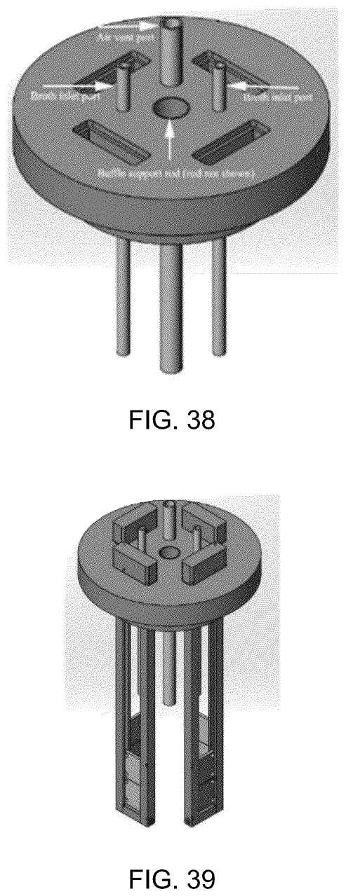

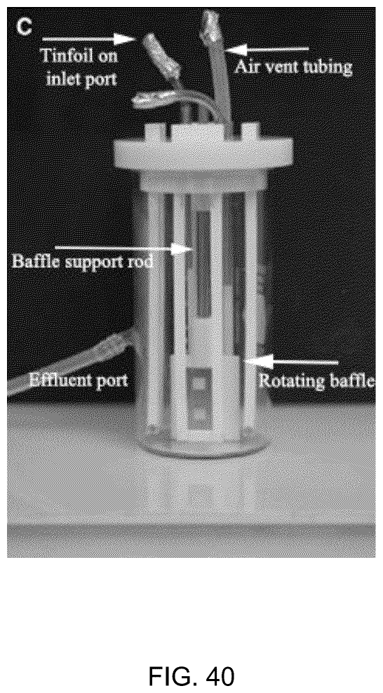

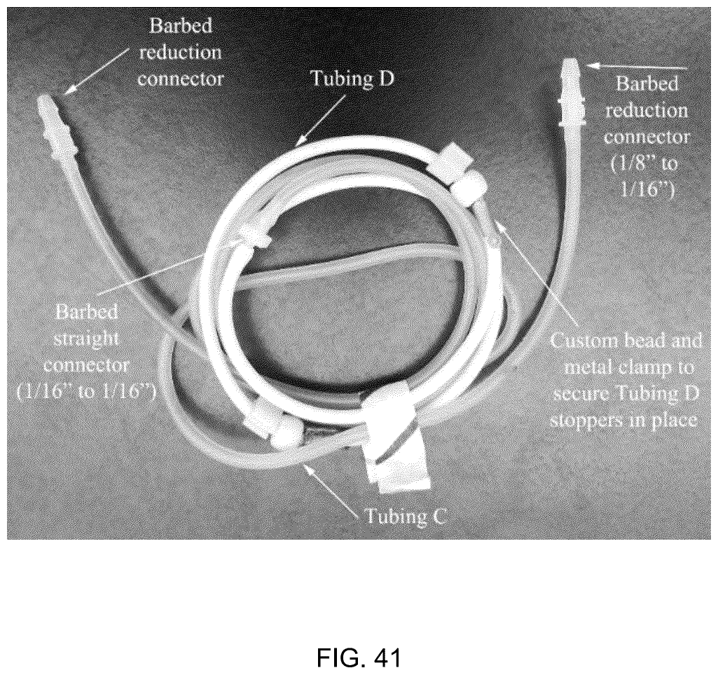

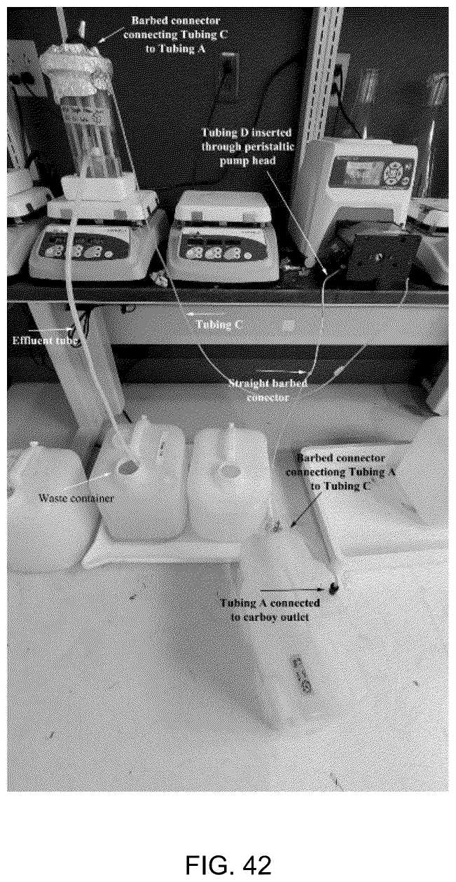

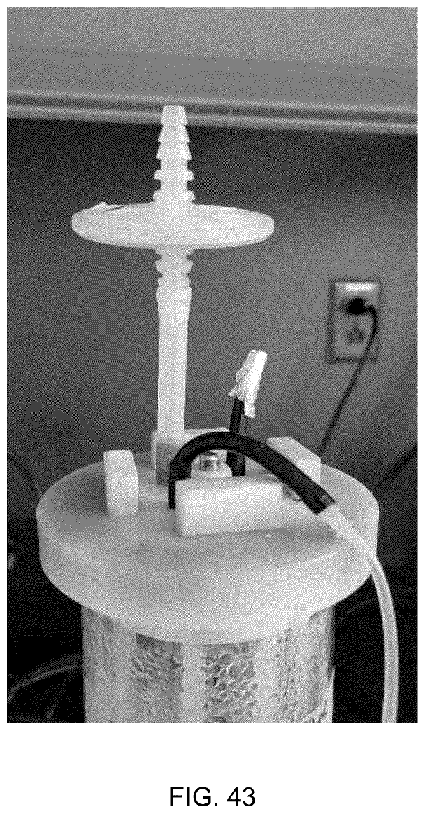

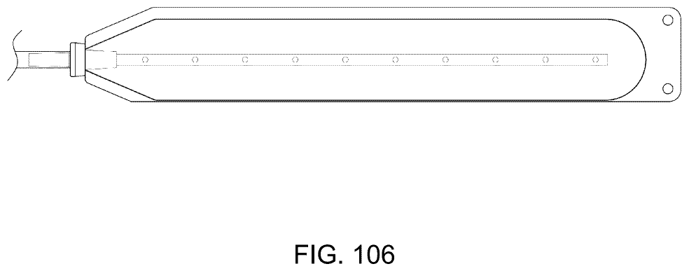

Iteration PES3 (FIGS. 22 & 23) and PES4 (FIG. 106) of the therapeutic device have also been tested for in vivo efficacy in treating biofilm implant-related infection. To do so, a sheep model of recalcitrant long bone open fracture infection was used. As part of this model, biofilms are first grown on the surface of 2 cm×2 cm×1.8 mm titanium squares that simulate fracture fixation plates (see FIGS. 35 & 36). Two plates are inserted into the holding arm of a modified CDC biofilm reactor (see FIGS. 37-39). The reactor unit is assembled and autoclaved and 500 ml of broth are added to the vessel (see FIGS. 40-43). The broth is inoculated with ˜7.5×10⁷ CFU of bacteria. Biofilms are grown on the titanium plates over a period of 48 h: the reactor is placed on a hot plate at 34° C. for 24 h (batch phase growth for bacteria to divide and condition the environment) after which a 10% solution of broth is flowed through the reactor at a rate of 6.9 ml/min for an additional 24 h (the latter growth phase allows biofilms to mature on the metal surface). Following biofilm growth, simulated fracture fixation plates are aseptically removed. An n=2 of the plates are reserved and quantified by vortexing, sonicating, and plating on agar using a 10-fold dilution series to determine the baseline of CFU/plate. The remaining n=6 plates (each reactor holds a total of n=8 simulated fracture fixation plates; see FIG. 39) are transported to an operating room for implantation in the right tibia of sheep.

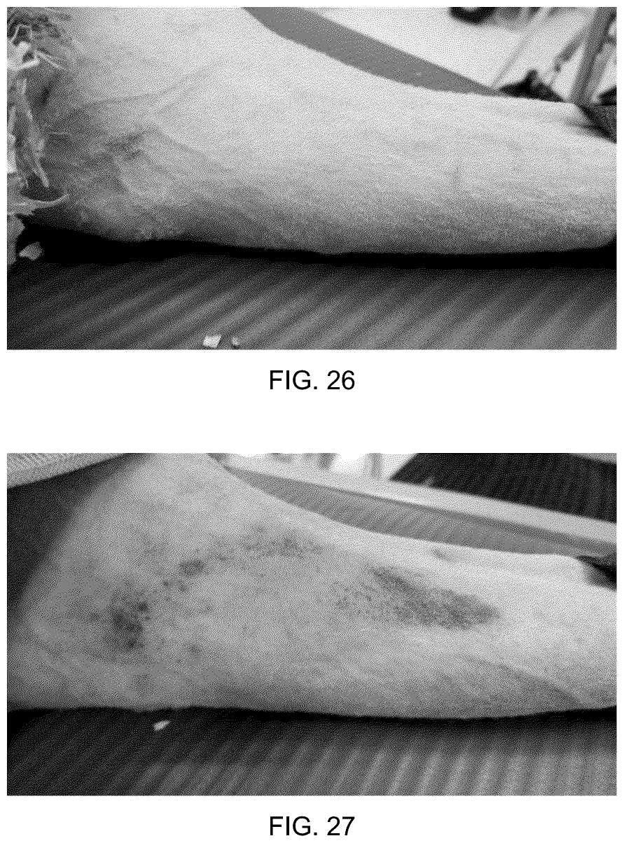

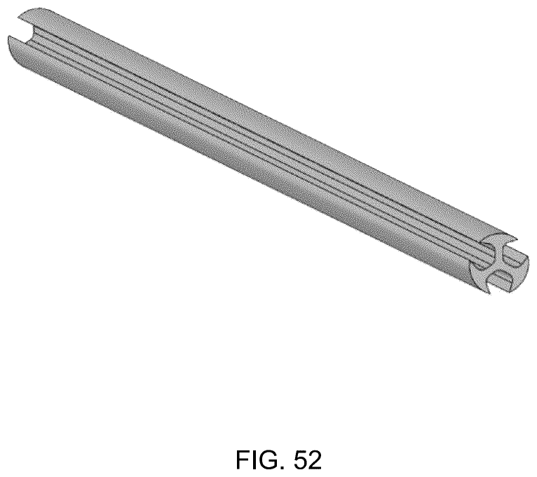

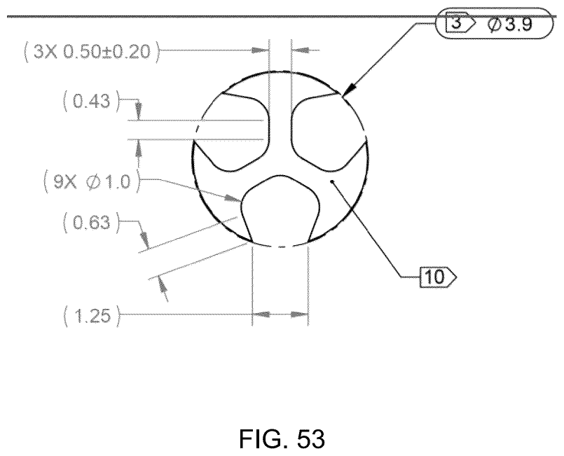

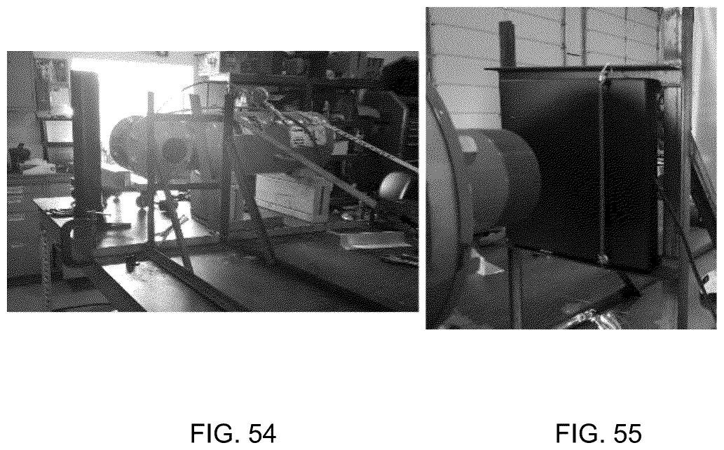

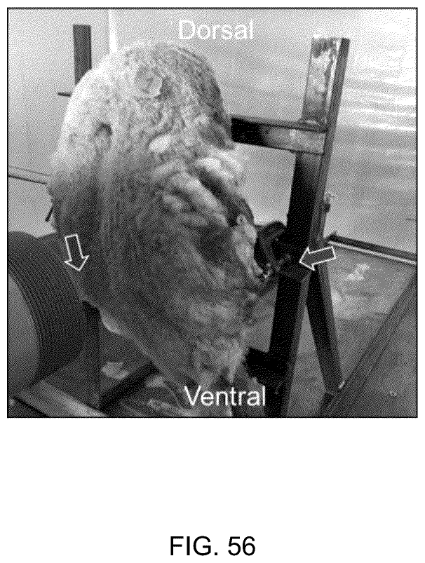

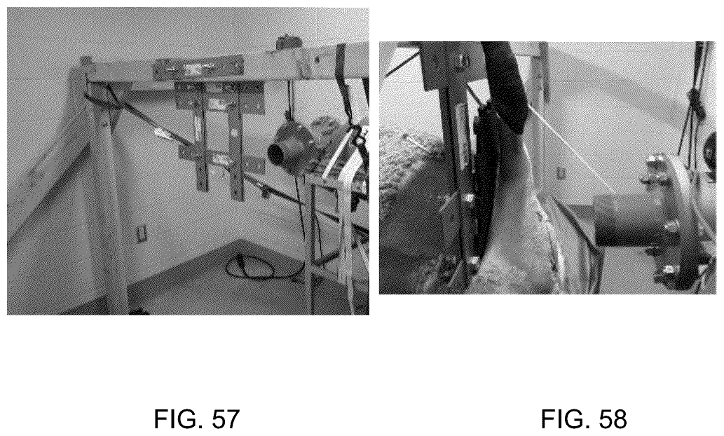

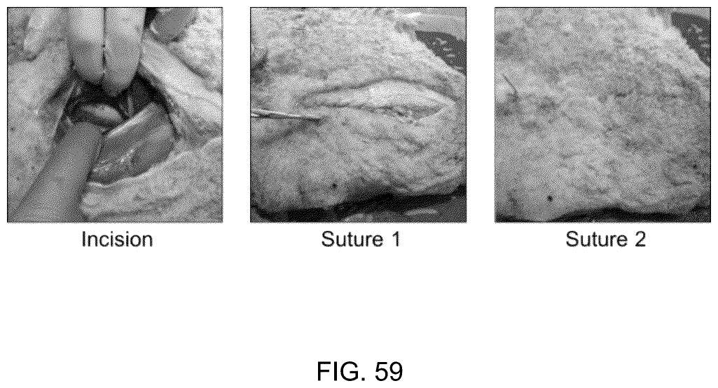

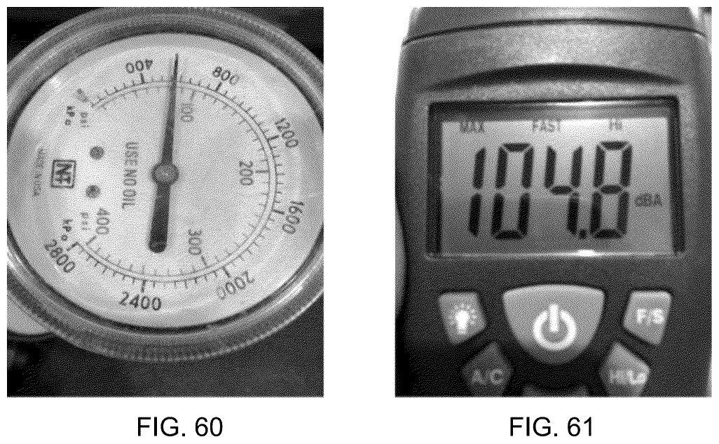

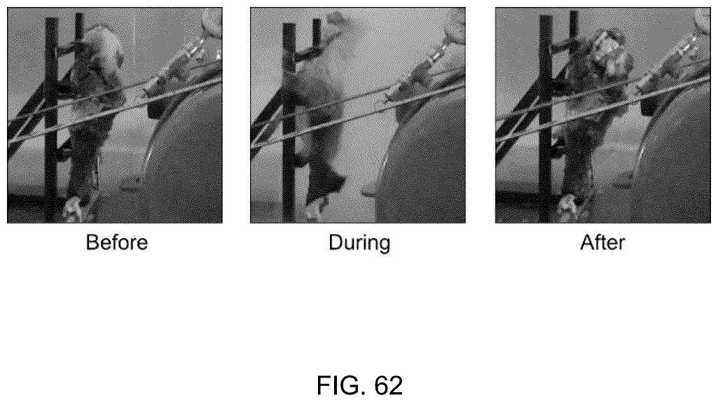

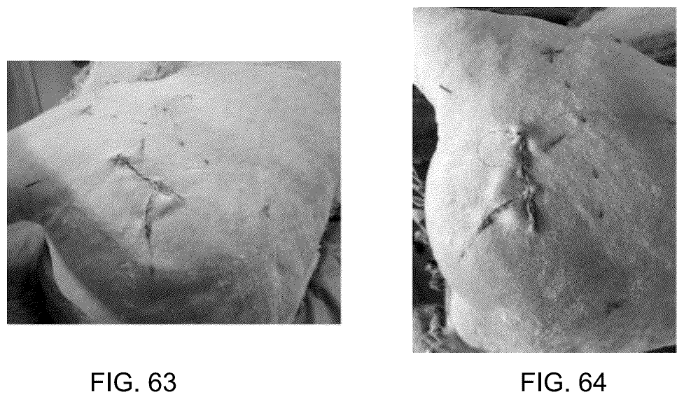

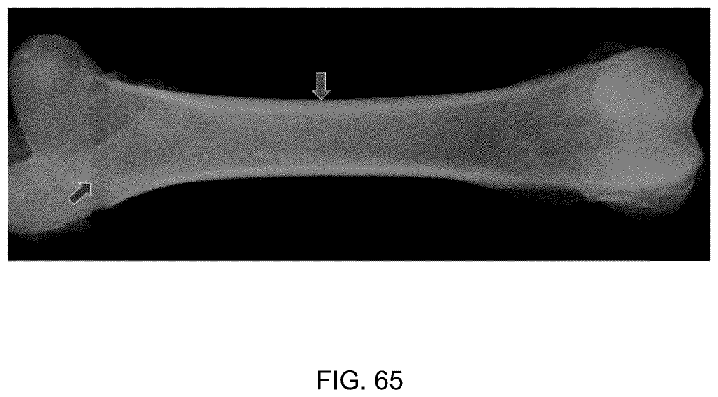

Immediately prior to surgery, the proximal medial aspect of a sheep's right tibia is blasted with an air impact device (AID; see FIGS. 52-64 & 94 for representative procedures of AID setup and outputs). The purpose of this blast is to create soft tissue trauma, which is consistent with a Type IIIB open fracture (see FIGS. 26 & 27). A Type IIIB open fracture is defined as having periosteal stripping, soft tissue trauma, and massive contamination. Following the AID blast, a sheep is prepped for surgery.

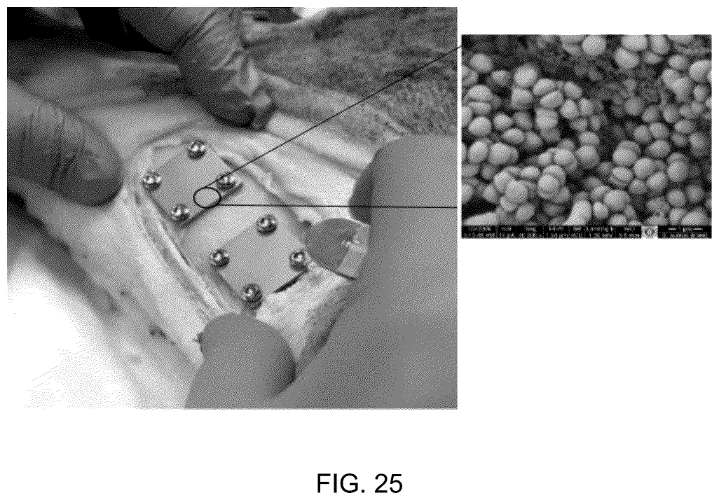

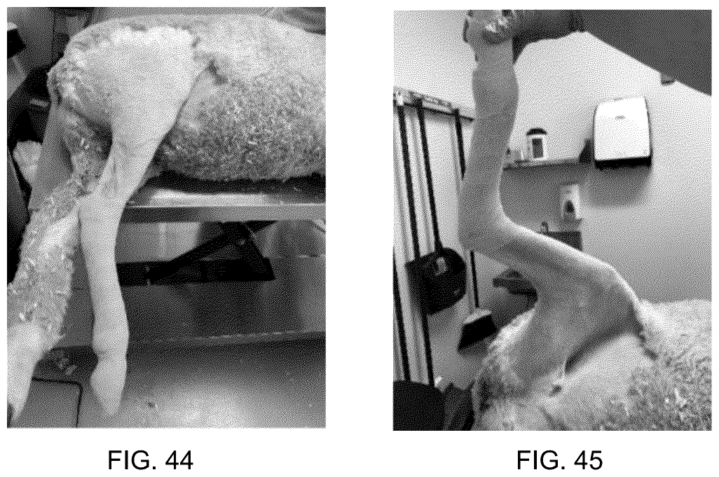

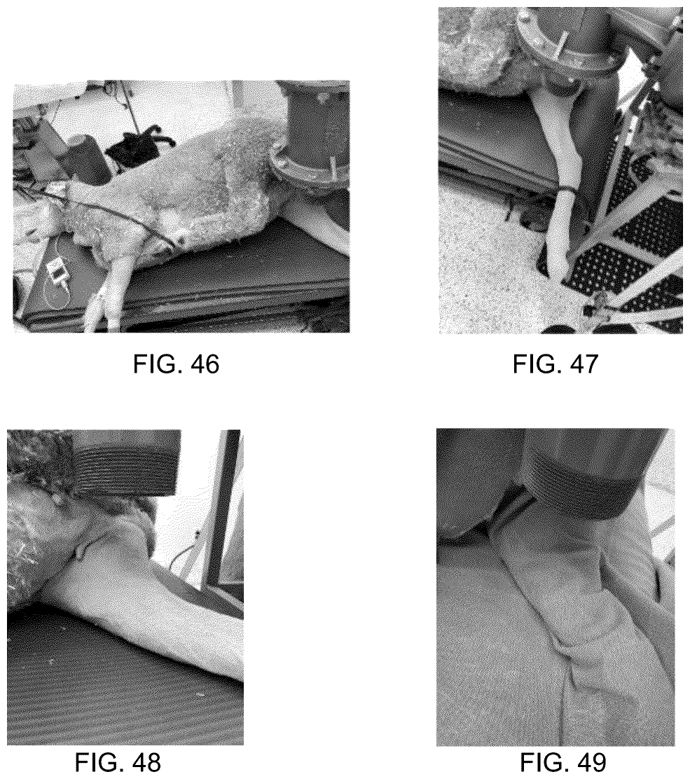

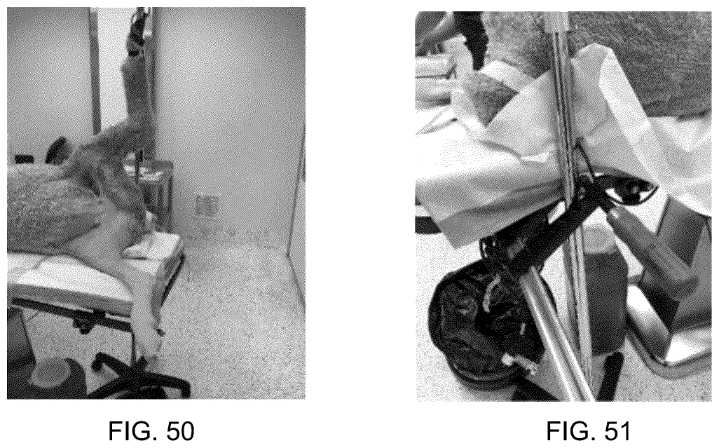

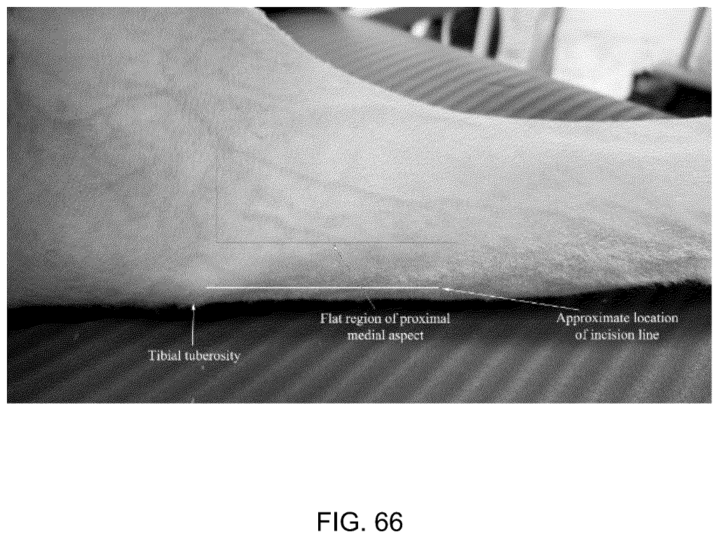

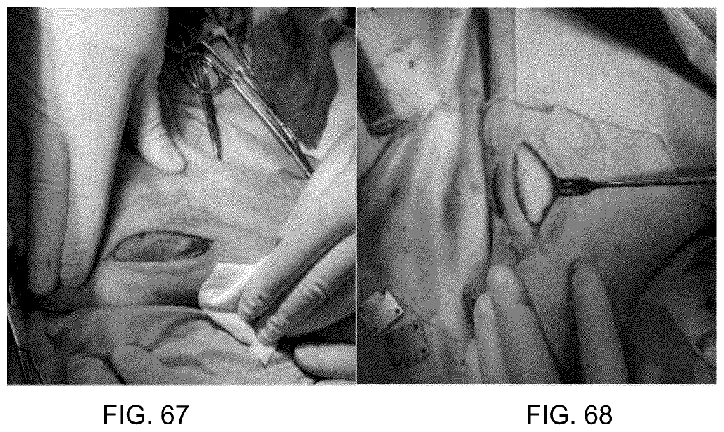

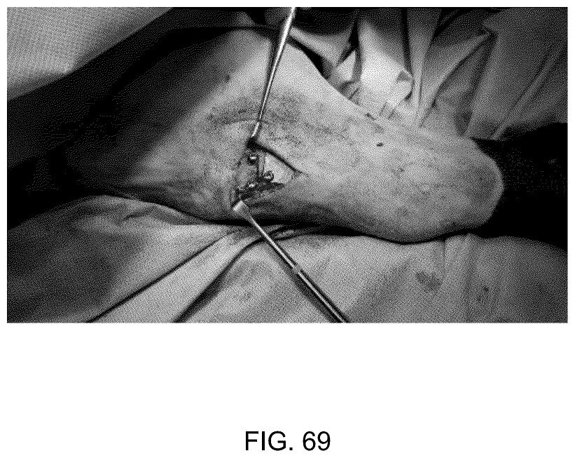

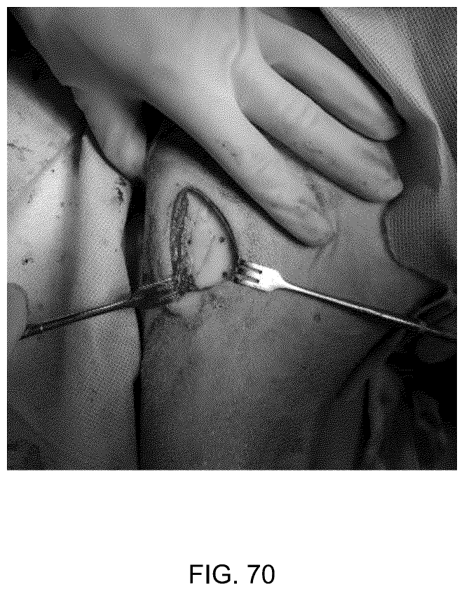

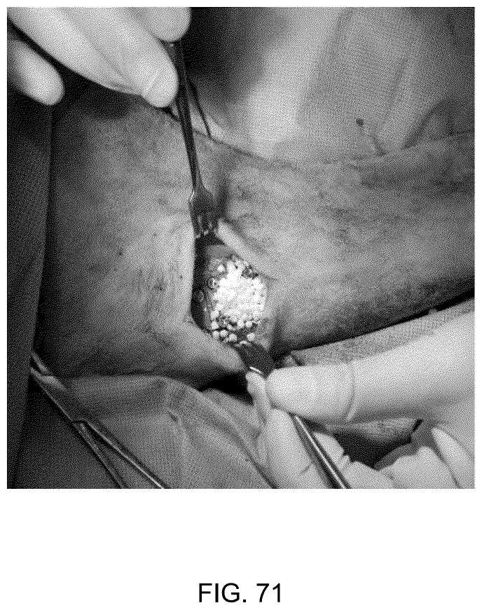

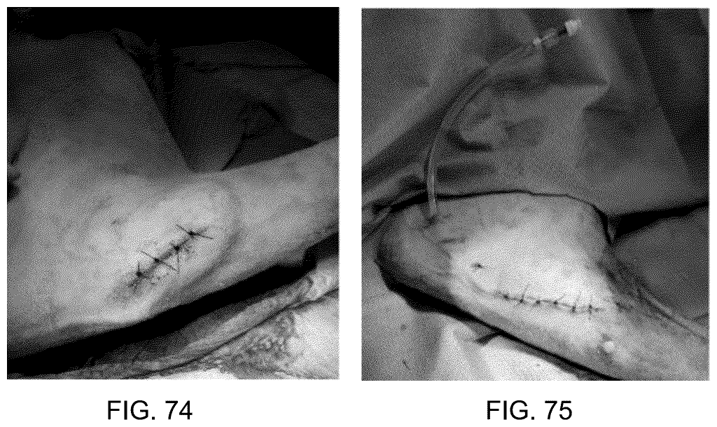

Surgical preparation and procedure are as follows: briefly, after the skin is prepped and sterile coverings placed (see FIGS. 44-51), an incision is made from the tibial tuberosity and down the shin, i.e., the front-most region of the tibia (see FIGS. 66 & 67). The incision length is approximately 5-7 cm. The fascia is moved/removed and a ˜2×5 cm area of periosteum is removed from the proximal medial aspect of the tibia using a periosteal elevator (see FIG. 68). Sterile simulated fracture fixation plates and sterile cortical bone screws are used to template the placement of the plates/screws (see FIG. 69). Once templated, an osteotomized fracture is created using a hand-held bone saw (see FIG. 70). The osteotomized fracture has a depth of ˜1-2 mm. Biofilm-ridden plates are then secured to the proximal medial aspect of the sheep tibia (sec FIG. 25). Sites are then ready to be sutured closed, or to receive a treatment (sec FIGS. 71-75).

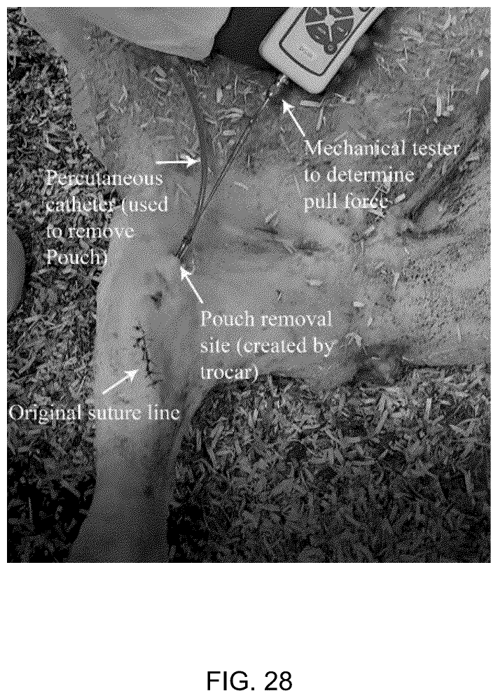

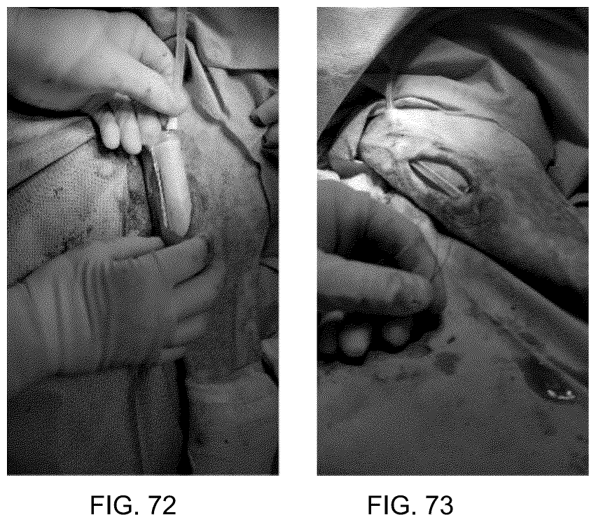

In the case of sheep that receive a therapeutic device, it is placed over the simulated fracture fixation plates and the surgical site closed (see FIGS. 72-75). The therapeutic device is filled and refilled daily with antibiotic solution and remains in place for 10 or 20 days, after which time it is removed (see FIG. 28). The same sheep model is also used to test the efficacy of clinical standards of care including systemic antibiotic therapy, antibiotic-loaded Stimulan beads, or antibiotic powders that are sprinkled into a wound site.

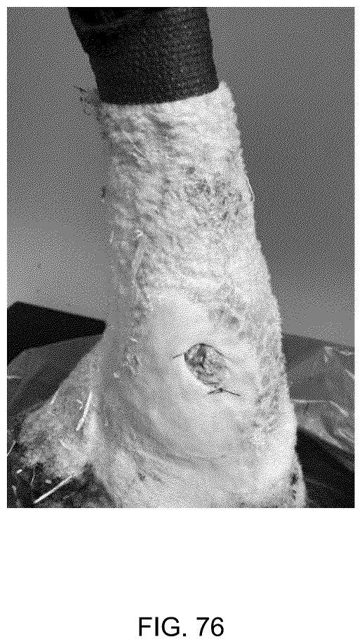

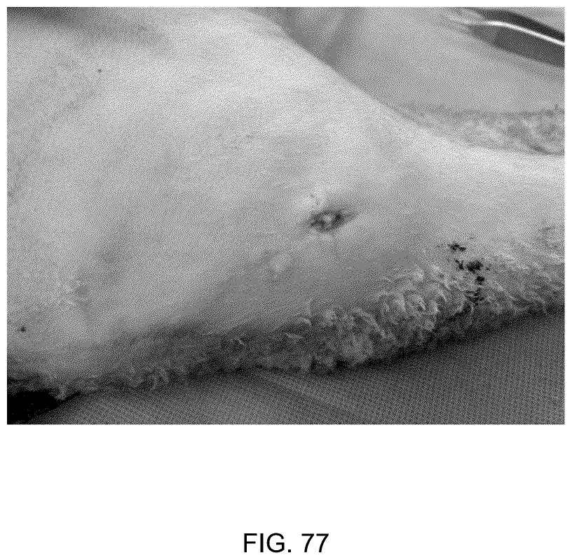

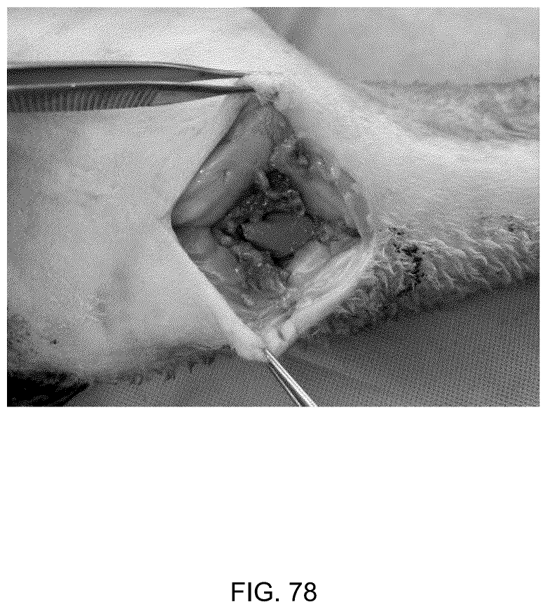

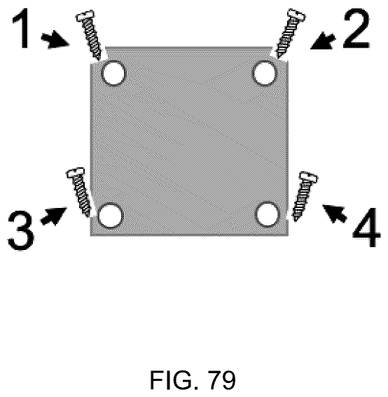

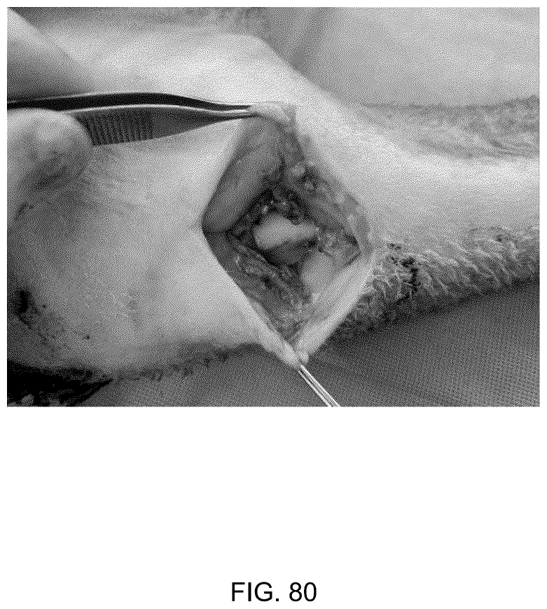

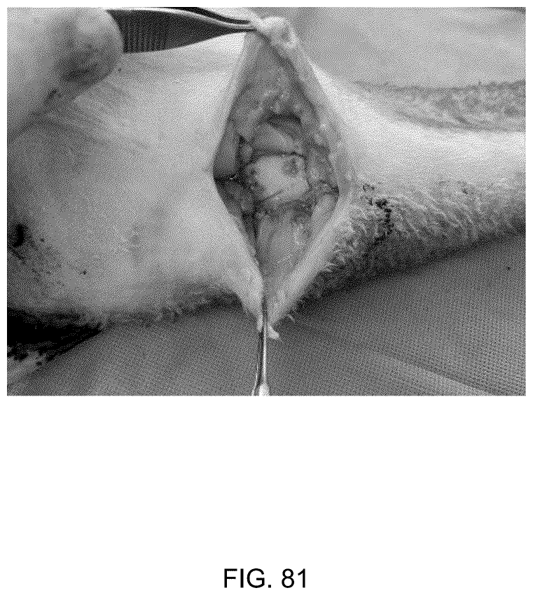

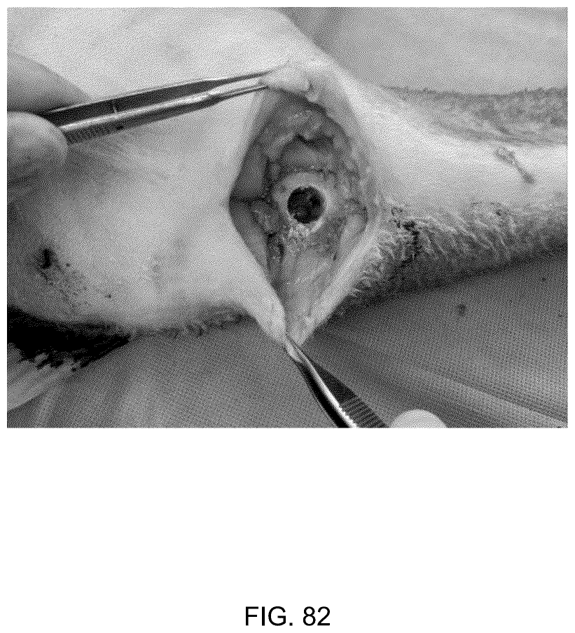

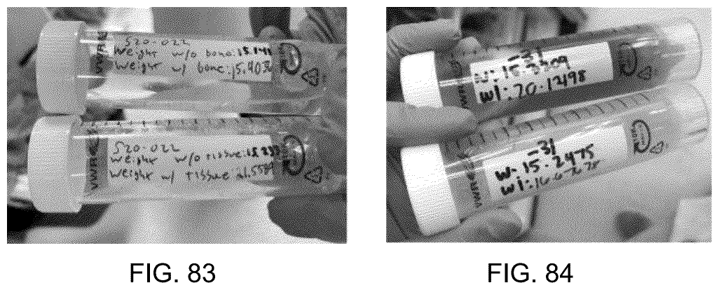

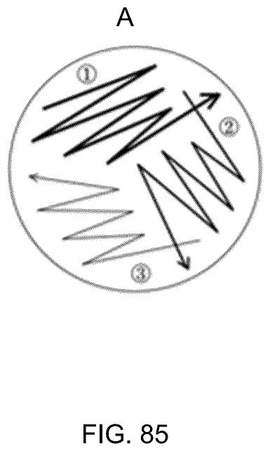

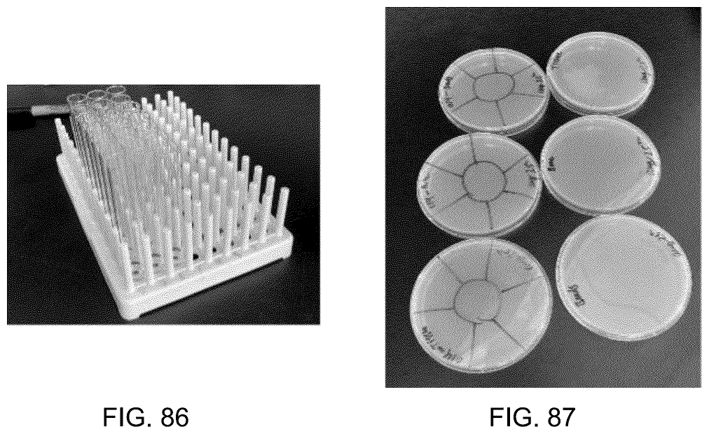

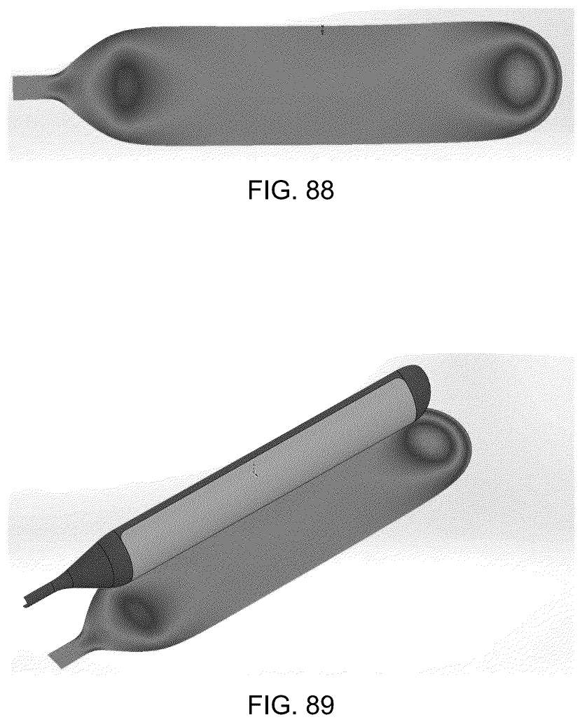

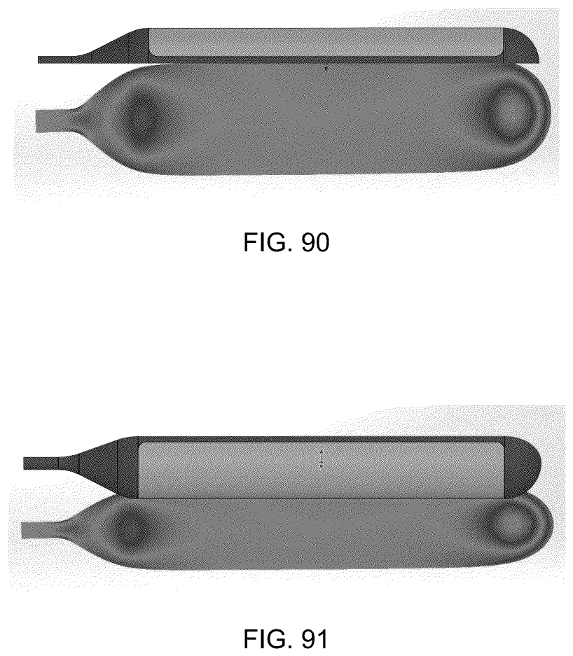

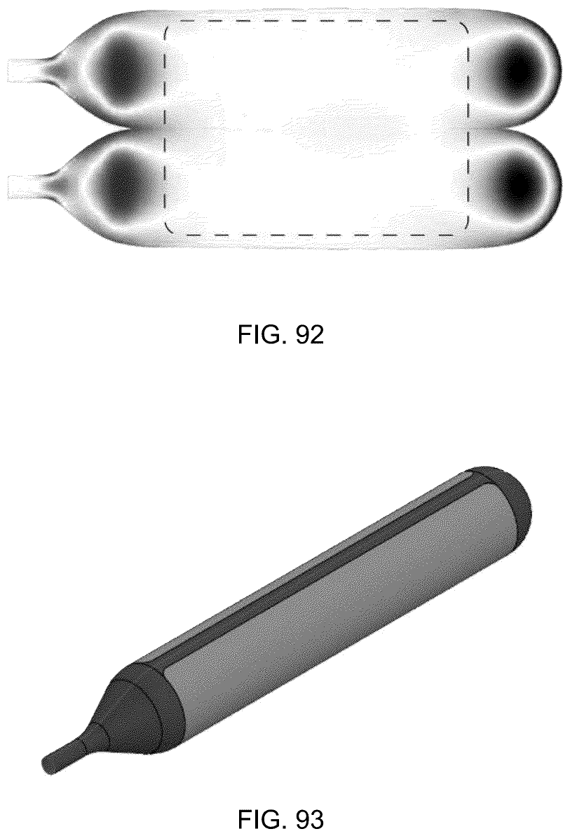

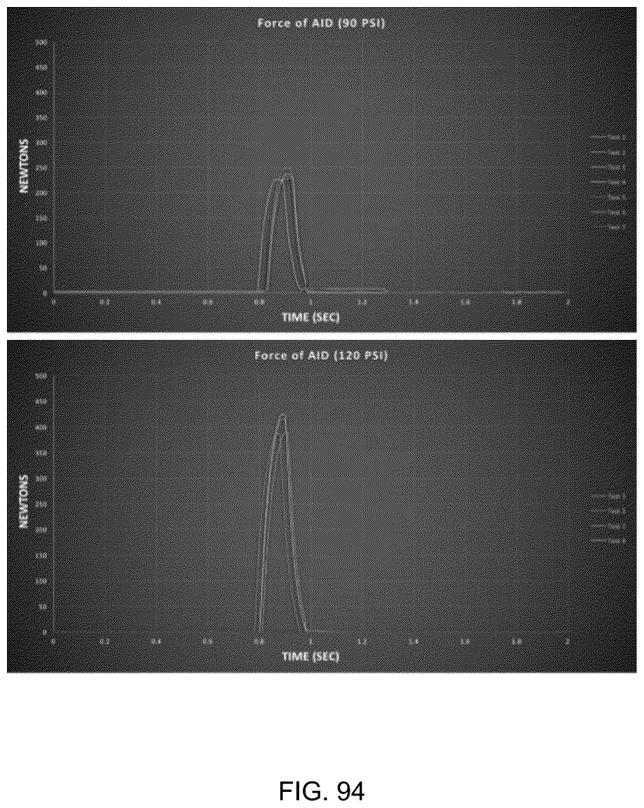

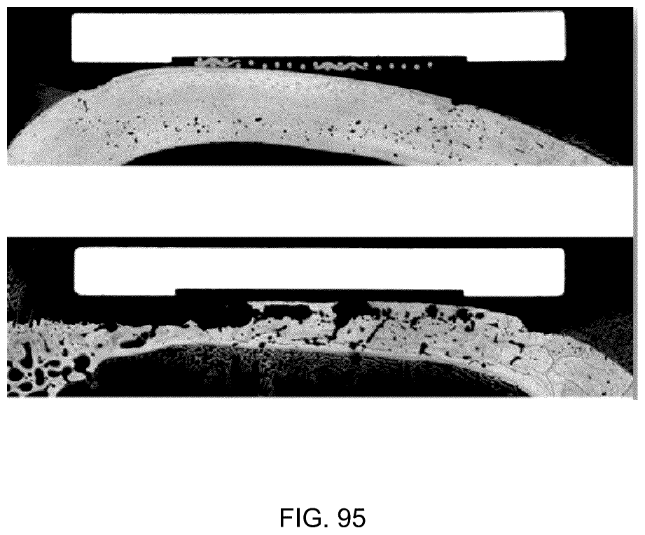

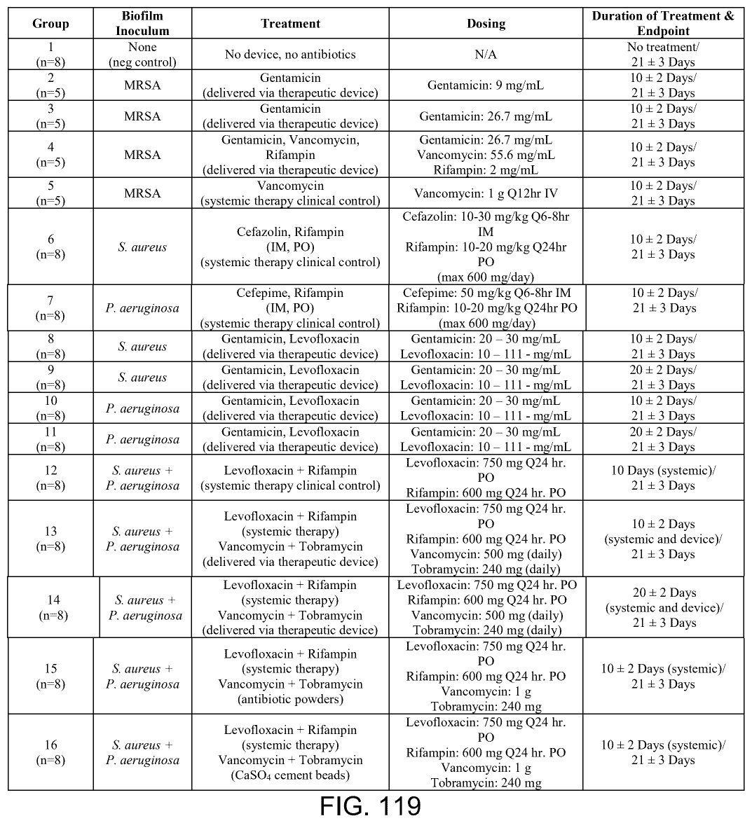

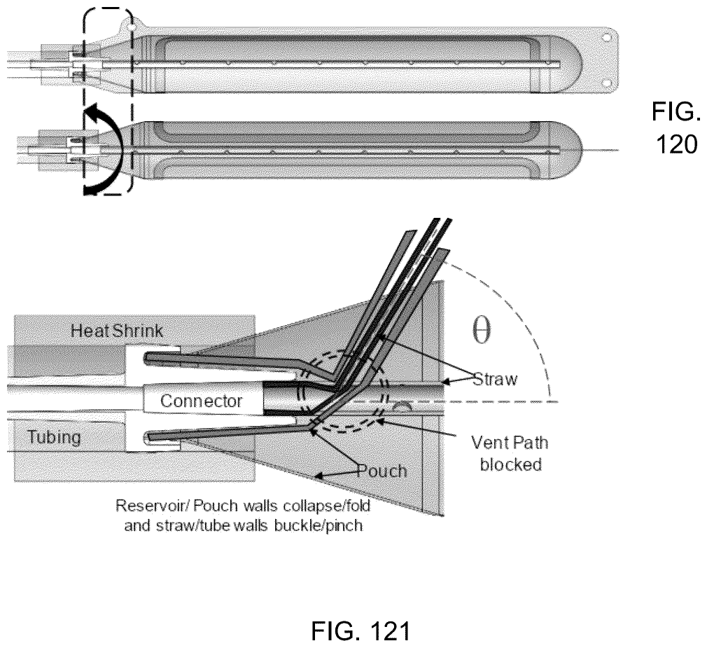

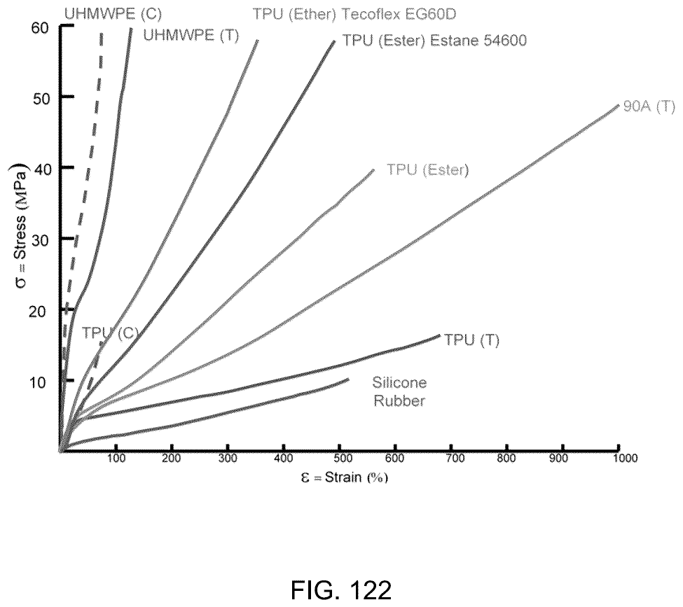

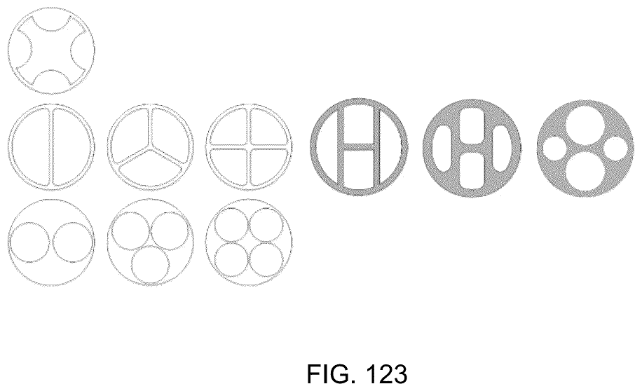

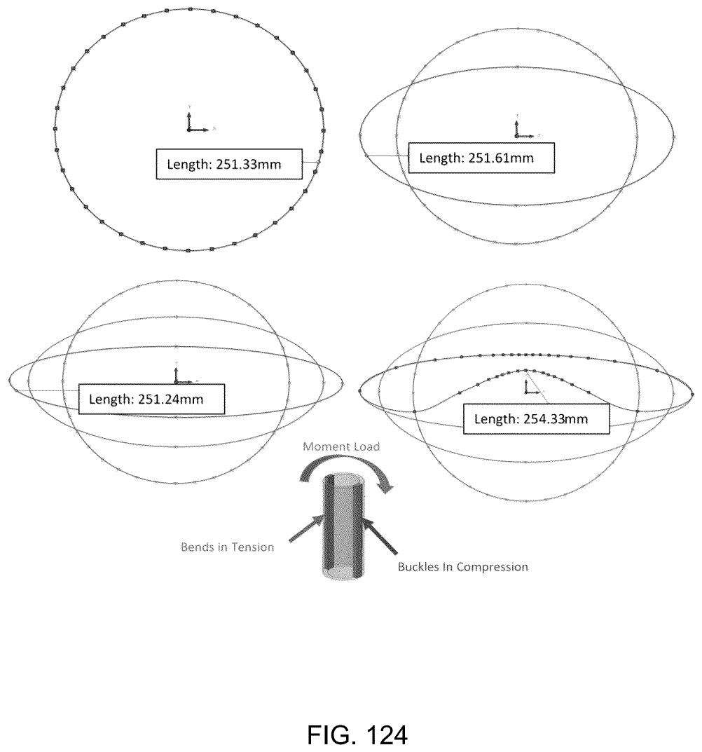

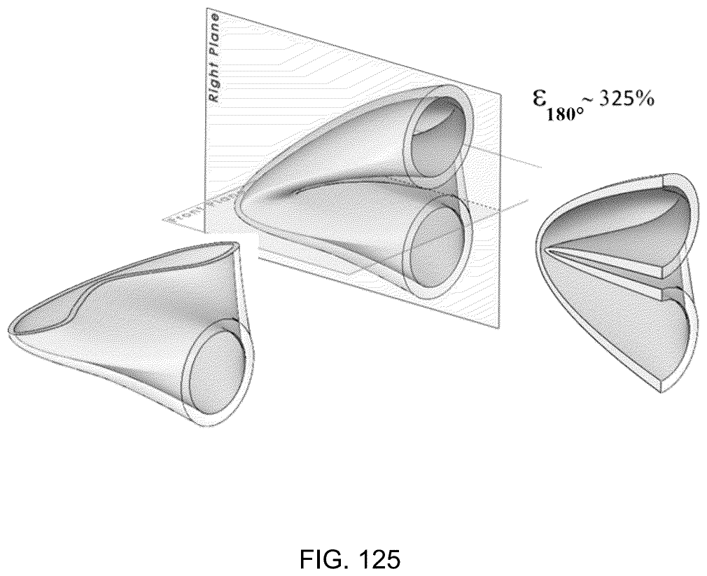

To date, the therapeutic device has been tested in n=63 sheep with a variety of antibiotic therapies (see Groups 2, 3, 4, 8, 9, 10, 11, 13, and 14 in table of FIG. 119) and compared to multiple standards of care including: gentamicin-loaded Stimulan beads (see FIG. 76), cefazolin and rifampin systemic therapy, cefepime and rifampin systemic therapy, levofloxacin and rifampin systemic therapy, vancomycin systemic therapy, or vancomycin and tobramycin powders sprinkled directly into wound sites (see Groups 5, 6, 7, 12, 15 and 16 in table of FIG. 119).

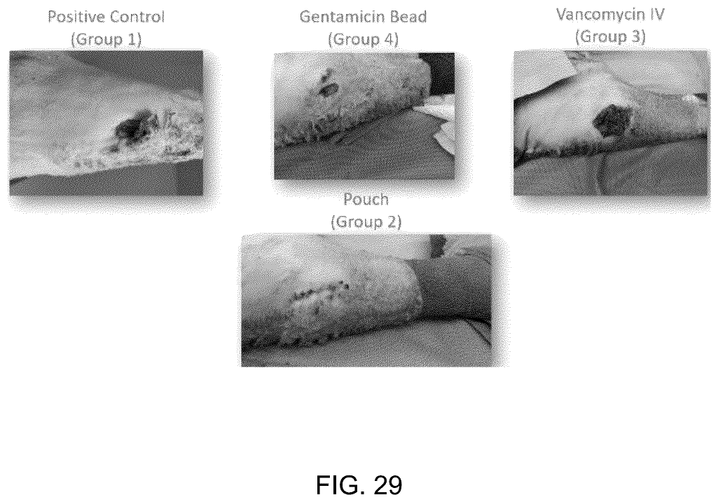

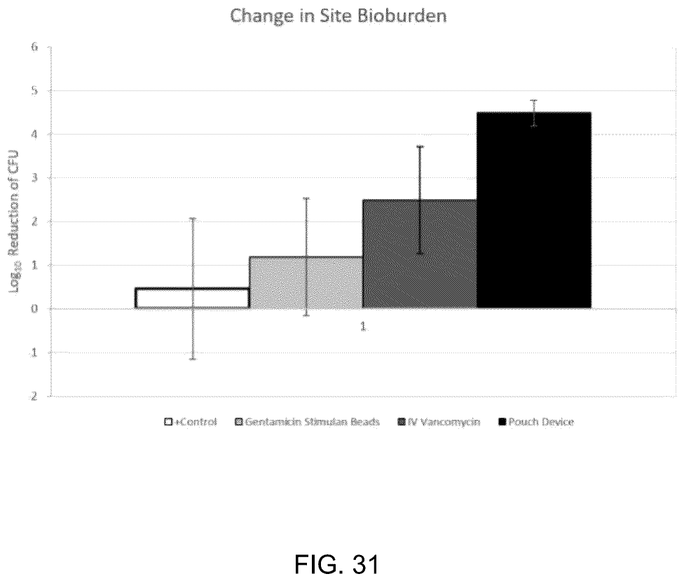

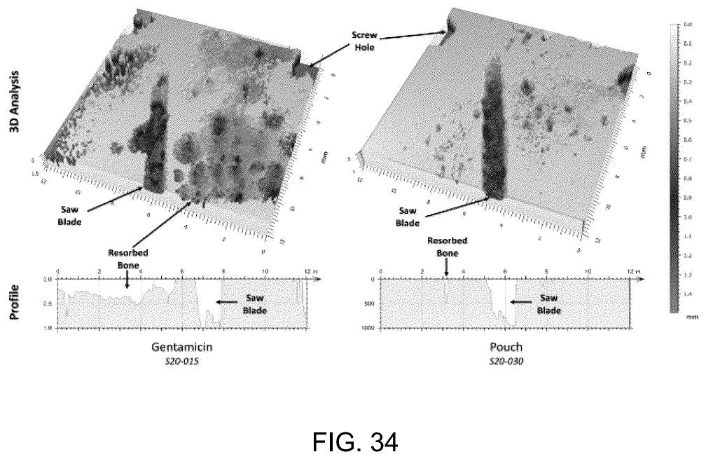

After treatment is completed in any given group, sheep are euthanized, legs are photographed, tissue samples and hardware are quantified microbiologically to determine the CFU/sample, and additional samples are analyzed by microCT and histologically to assess bone response (see FIGS. 29-34 & 76-87).

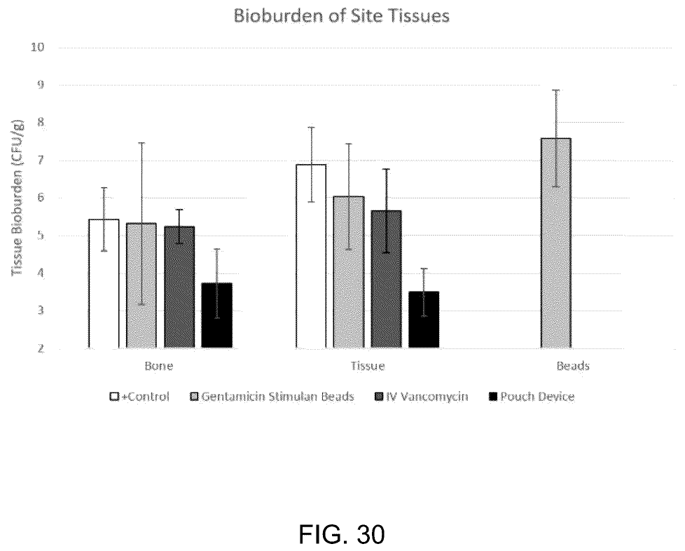

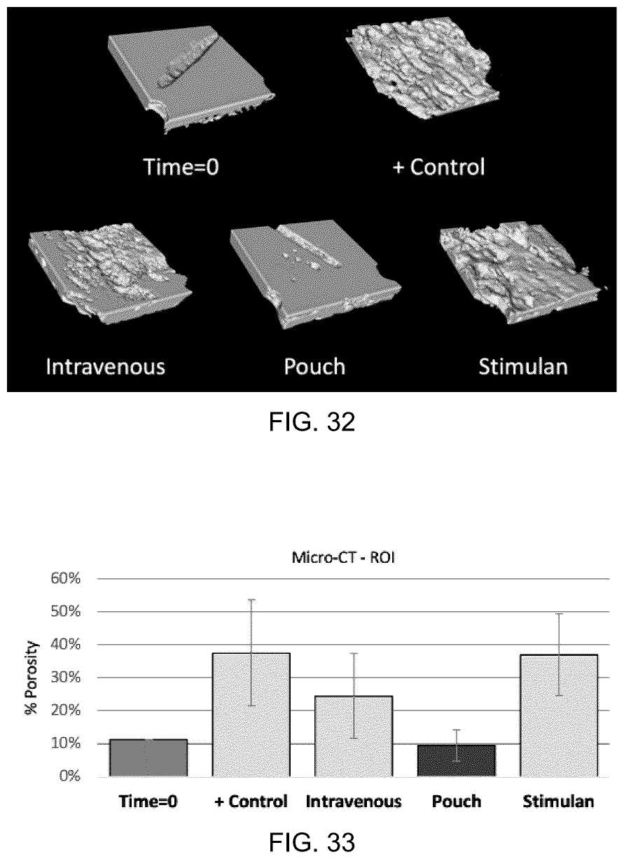

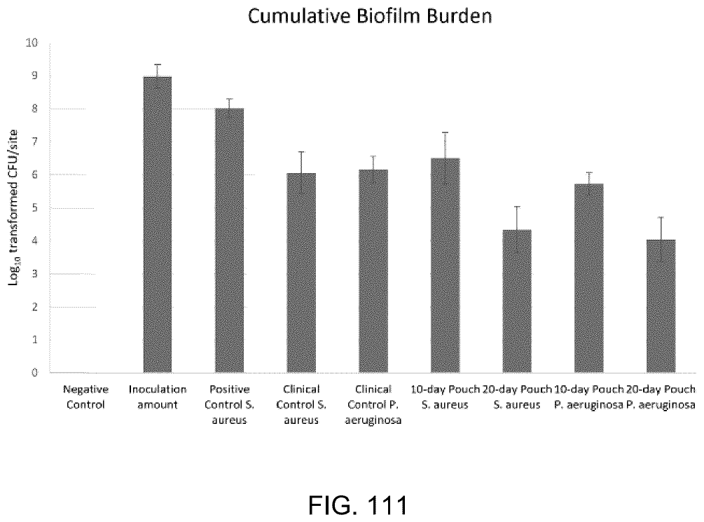

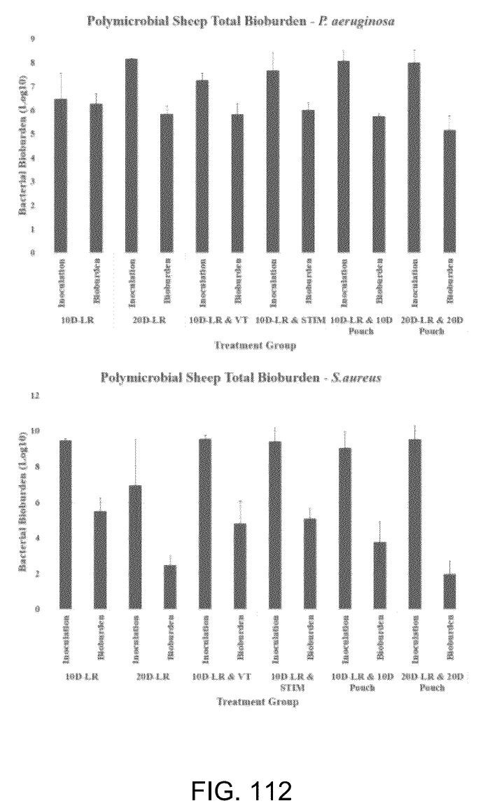

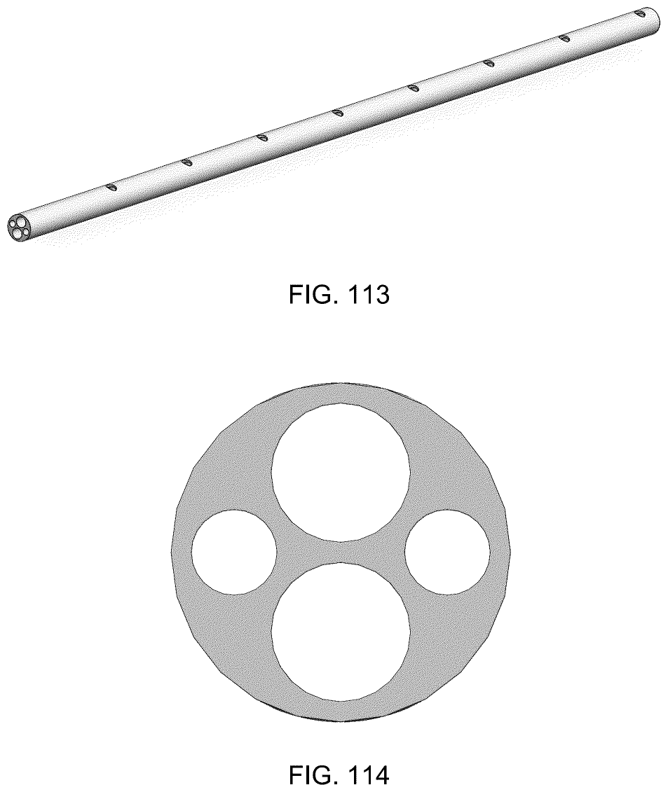

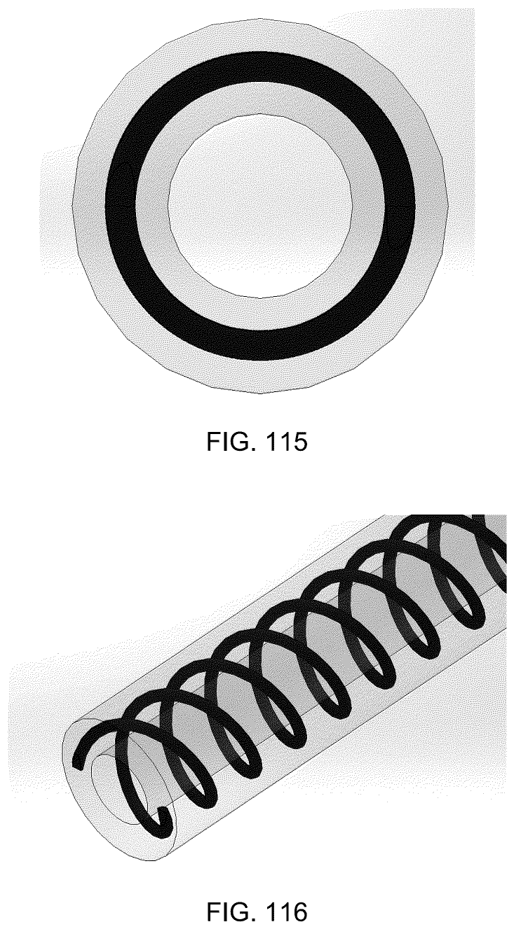

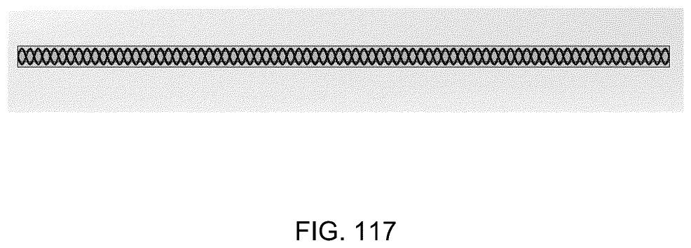

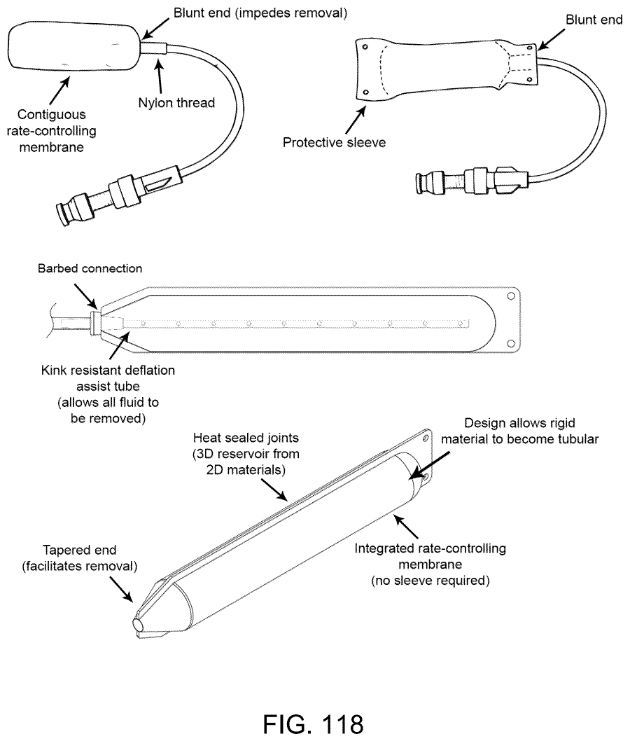

Data indicate that the therapeutic device reduces biofilms by a minimum of 10 and up to 10,000 times more than clinical standards (see FIGS. 30, 31, 111, & 112). Furthermore, microCT and histological analyses show that sites treated with the therapeutic device have significantly fewer signs of infection than any other treatment group (sec FIGS. 32-34 & 111-112).

During animal studies described herein, it was determined that the devices disclosed herein, especially the reservoir and the tube of the device, may twist, kink, or fold on itself or against a portion of the animal's body, often restricting or stopping the flow of the one or more therapeutic agents to the reservoir of the device and animal, causing the device to not function properly or as intended. Oftentimes, the disruption in the flow of the one or more therapeutic agents was at or near the neck area of the device where the one or more therapeutic agents entered into the reservoir of the device. Numerous experiments using the devices disclosed herein demonstrated that during use in an animal, the fluid pathway of the one of more therapeutic agents, was constricted, often at or near the neck area of the device, even though there was no visible or otherwise discernible change to the condition of any component of the therapeutic device, including but not limited to, the tubing near the neck or reservoir of the device. Constriction and/forces to the device can hinder the transfer of therapeutic agents in and out of the reservoir of the device and to the animal it is being administered to.

Accordingly, various styles, shapes and configurations of different tubes having various different characteristics and shapes were tested to determine which of them, if any, would allow the devices disclosed herein to function as intended and avoid the flow of the one or more therapeutic agents in the device from being constricted anywhere in the device. Based on the results disclosed herein, a single lumen tube through the neck of the pouch was found to be an unsuitable remedy to the problem. Evaluations were conducted of many alternate materials and various multi-lumen configurations, and it was found that tube having more than two lumens performed better and four-lumen tubes consistently remained unblocked even though the devices underwent severe deformations and forces that were well beyond what one would expect the devices of the present disclosure to encounter in normal applications using the devices of the present disclosure.

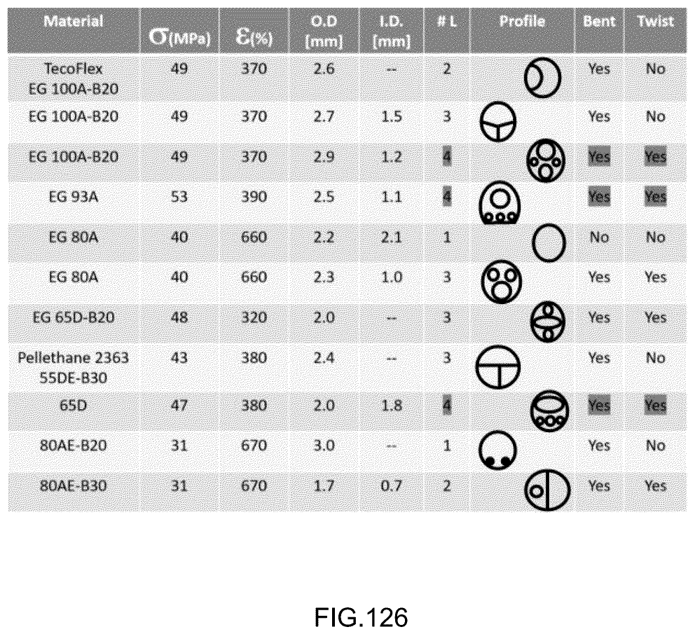

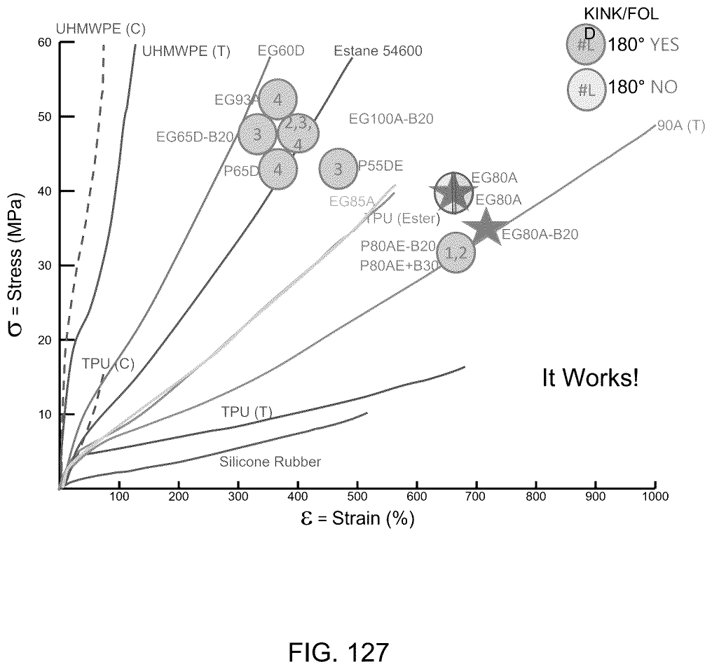

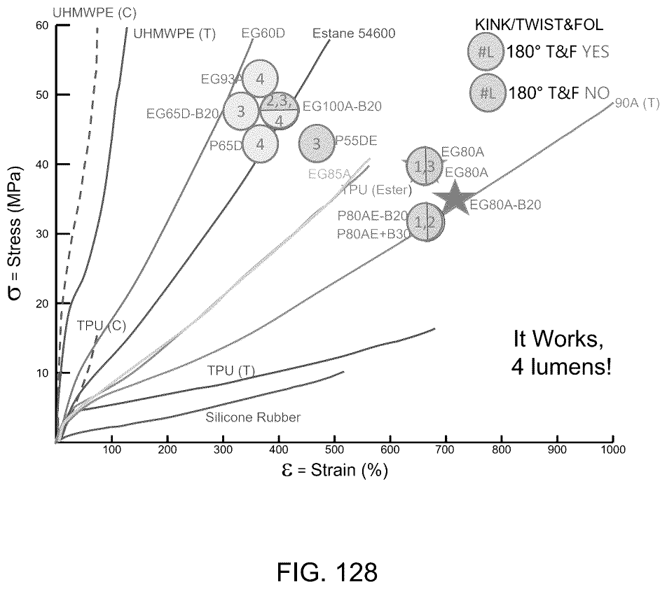

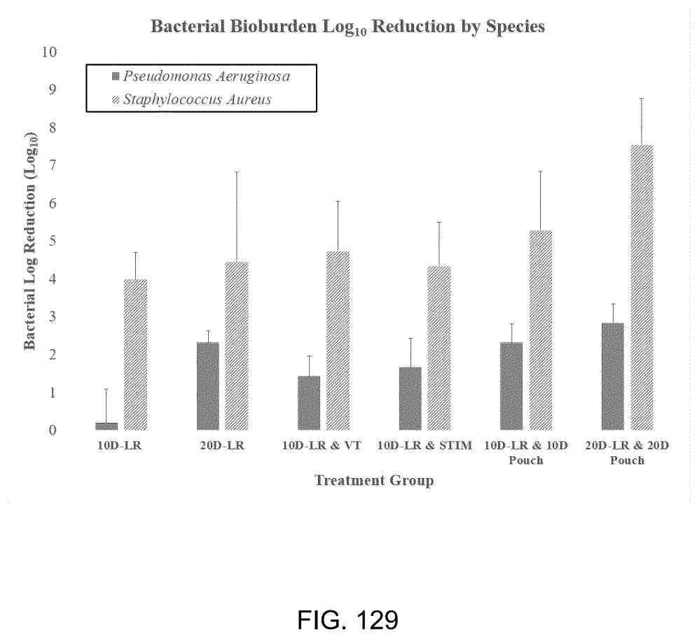

A variety of available materials with various lumen configurations and layout patterns were obtained and tested. The lumen and tube characteristics were selected based on the material characteristics of ultimate stress (σ_{U}) and ultimate strain (ε_{U}). These were tested by attaching each, individually, to a 25-cc syringe filled with water and dispensing the fluid through the tube to gauge case of fluid flow. Next the tubes were folded 180° onto themselves and the syringe dispensing repeated. If water could transfer through the tube without a moderate increase in applied pressure it was recorded as a success (yes). If fluid did not flow or required a significant increase in applied pressure it was recorded as a failure (no). This testing was repeated on the tubes with the addition of a 180° twist applied prior to the 180°-fold over and results recorded. FIGS. 126-128 show and describe the materials and different lumen tested, their characteristic, the number of lumens, a simplified sketch of the lumen layout, and the syringe dispense results for a 180° bend (fold) and the combination of 180° twist and fold tests that were performed on the various lumen shown and described herein.

As shown by the results in FIGS. 126-128, all of the multi-lumen samples passed the 180°-Fold test. Only one single lumen sample failed. Refer to FIGS. 126-128 for the data and graphical representation of the results of the test covering the multi-lumen tube. As the results demonstrate, several multi-lumen configurations passed the 180°-Twist combined with a 180°-Fold test. Only the four-lumen configurations consistently passed.

The results of tests on prior versions of the devices show that a flexible single lumen tube under bending or twisting loads can deform to the point it no longer allows fluid to transfer through the tube, which renders the device as useless and could cause damage to the animal it is inserted into. By modifying the cross-section of the tube with a support structure it can be made to withstand severe deformation and continue to function as intended to allow the unrestricted flow of the one or more therapeutic agents. Multi-lumen tubes, particularly configured to create an internal web and flange design (H-beam) will not fail. By selecting a material that has a low modulus of elasticity and high strain limit, the tube will stretch yet not tear as the cross members prevent the tube walls from closing the fluid path. Certain cross-section designs shown in the results of FIGS. 126-128 demonstrate that certain multi-lumen tubes were able withstand any loading orientation regardless of the neutral axis location. Those multi-lumen tubes can be used to obtain a non-closable fluid pathway for the devices disclosed herein.

In another aspect of at least one embodiment of the present disclosure, as shown in FIG. 130J, a heat seal weld joining the two subassemblies in FIG. 130H. Previous versions of the device used a custom fitting and glue to join these two components. The embodiments of the present disclosure eliminated the need for either the glue or the custom fitting by use of a seal (e.g., heat seal), which eliminated the need for a specialized fitting and drastically increased the strength of that joint as shown in FIG. 133, from ˜50 N of force to about 225 N of force before the device failed. It should be appreciated that the devices of the present disclosure can be formed using any known way of attachment, seal or manufacture, including but not limited to heat or other type of seal, welding, ultrasonic welding, and solvent welding.

Benchtop outcome testing was performed to test the various deflation technologies and ensure the devices shown and described herein provide, among other things, a reliable and repeatable deflation and inflation of the devices that have been surgically implanted into an animal. The results of the benchtop outcome testing showed that when the device was severely kinked, bent and/or contorted, certain device configurations and attributes shown and described herein enabled more efficient, reliable and repeatable removal and refilling of the therapeutic agents that were removed and refilled using a syringe. Accordingly, the results of the benchtop outcome testing demonstrated that the configurations and attributes shown effectively mitigate and possibly eliminate device failure and user error, thereby obviating the need for additional surgery to remove and/or replace the device shown and described herein.

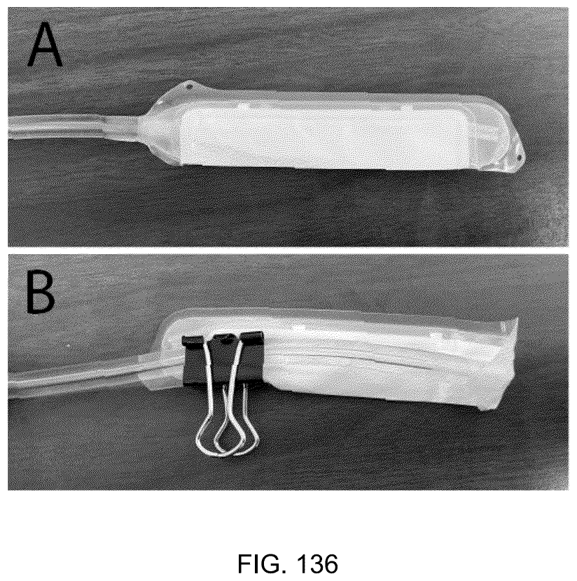

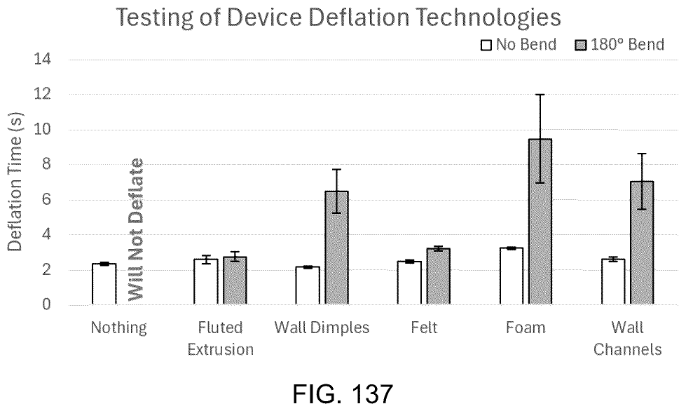

The benchtop testing was performed on the devices shown in FIG. 136 and described herein by filling the devices of the present disclosure with 10 ml of DI water, drawing a vacuum on a connected 10 ml syringe and recording the time it took for complete removal most if not all of the 10 ml of DI water from the device shown in FIG. 136. Testing was performed with the devices shown in FIG. 136 in both their natural position and bent at the neck by 180° as shown in FIG. 136. The results of the benchtop test are shown and described in FIG. 137. As the results demonstrate that the device of the present disclosure would not deflate without any deflation technology. The results shown and described in FIG. 137 demonstrate that providing a fluted extrusion allows the device to be deflated with the fastest deflation time when the device is bent at the neck by 180°. The results also demonstrated that using felt in the device resulted in the second fastest deflation time when the device was bent at the neck by 180°.

In yet another embodiment of the present disclosure, a thin felt layer, either bound to the flat sheet components, or attached to the tubing, can be used. It should be appreciated that this maintains a fluid path if the device is contorted or kinked while inside the animal. The felt layer is used to assure an air path remains open for vacuuming or removing the therapeutic compositions in the device.

Although the invention has been described in detail with reference to certain preferred embodiments, variations and modifications exist within the scope and spirit of one or more independent aspects of the invention as described.

What is claimed is:

1. A method of eradicating biofilm and planktonic bacteria in an animal, the method comprising,
providing a therapeutic delivery device comprised of at least a semi rigid body defining at least one internal reservoir for holding one or more therapeutic agents that are to be delivered to an animal;
a porous rate-controlling membrane located in the internal reservoir that controls the rate of release of the therapeutic agent to the animal; and
a fluted extrusion refilling tube in fluid communication with the reservoir that facilitates inflation or deflation of the internal reservoir with the one or more therapeutic agents;
wherein at least the edges of the porous rate-controlling composite membrane are sealed or clad with the at least a portion of the semi rigid body to form a contiguous, fully integrated therapeutic device.

2. The method of claim 1, wherein the device further comprises a fluted extrusion in fluid communication with the at least one internal reservoir

3. The method of claim 2, wherein the fluted extrusion is heat-sealed, radio frequency-welded, ultrasonic-welded, solvent-welded, glued, compression fit or plasma bonded to at least a portion of the semi-rigid body, the porous rate-controlling membrane or the refilling tube.

4. The method of claim 2, wherein the fluted extrusion is in fluid communication with the at least one internal reservoir.

5. The method of claim 1, wherein the porous rate-controlling composite membrane is comprised of fibrous polyethylene terephthalate.

6. The method of claim 1, wherein the porous rate-controlling composite membrane is comprised of polyethersulfone and a fibrous polyethylene terephthalate.

7. The method of claim 1, wherein the device further comprises the at least semi rigid body having one or more monotonically tapered suture anchors.

8. A method of eradicating biofilm and planktonic bacteria in an animal, the method comprising,
providing a therapeutic delivery device comprised of at least a semi rigid body having one or more monotonically tapered suture anchors and defining at least one internal reservoir for holding one or more therapeutic agents;
a porous rate-controlling composite membrane located in the internal reservoir that controls the rate of release of the therapeutic agent to the animal; and
a tri-fluted extrusion refilling tube in fluid communication with the reservoir that facilitates inflation or deflation of the internal reservoir with the one or more therapeutic agents;
wherein at least the edges of the porous rate-controlling composite membrane are sealed or clad with the at least a portion of the semi rigid body; and
wherein the tri-fluted extrusion is heat sealed, RF welded, ultrasonic welded or solvent welded to the at least the semi rigid body to form a contiguous, fully integrated therapeutic device.

9. The method of claim 8, wherein the porous rate-controlling composite membrane is comprised of polyethersulfone.

10. The method of claim 8, wherein the porous rate-controlling composite membrane is comprised of fibrous polyethylene terephthalate.

11. The method of claim 8, wherein the porous rate-controlling composite membrane is comprised of polyethersulfone and a fibrous polyethylene terephthalate.

12. A method of eradicating biofilm and planktonic bacteria in an animal, the method comprising,
providing a therapeutic delivery device comprised of at least a semi rigid body defining at least one internal reservoir for holding one or more therapeutic agents; and
a porous rate-controlling composite membrane located in the internal reservoir that controls the rate of release of the therapeutic agent to the animal;
one or more dimples, fluted extrusions or channels located in at least a portion of the internal reservoir;
wherein at least the edges of the porous rate-controlling composite membrane are sealed with the at least a portion of the semi rigid body to form a contiguous, fully integrated therapeutic device; and
wherein the one or more dimples, fluted extrusions or channels of the internal reservoir allow the path of the therapeutic agents to remain open even if the device is kinked or twisted upon itself.

13. The method of claim 12, wherein the device further comprises a lock fitting and lock connector.

14. The method of claim 12, wherein the device further comprises a trocar.

15. The method of claim 12, wherein the porous rate-controlling composite membrane is comprised of polyethersulfone.

16. The method of claim 12, wherein the porous rate-controlling composite membrane is comprised of fibrous polyethylene terephthalate.

17. The method of claim 12, wherein the porous rate-controlling composite membrane is comprised of polyethersulfone and a fibrous polyethylene terephthalate.

18. The method of claim 12, wherein the device further comprises one or more dimples or channels located in close proximity to the end of the device where the one or more therapeutic agents are filled or refilled.

19. The method of claim 12. wherein the device further comprises a fluted extrusion in fluid communication with the at least one internal reservoir.

20. The method of claim 12, wherein the rate-controlling composite membrane is a fibrous material or mesh in the at least one internal reservoir.
